(19) United States
(12) Patent Application Publication
(10) Pub. No.: US 2022/0056411 A1
(43) Pub. Date: Feb. 24, 2022

(54) METHODS FOR ISOLATING CD8+ SELECTED T CELLS

(71) Applicant: Immatics US, Inc., Houston, TX (US)

(72) Inventors: Mamta KALRA, Houston, TX (US); Gagan BAJWA, Houston, TX (US); Thomas LUCAS, Houston, TX (US); Ali MOHAMED, Houston, TX (US); Steffen WALTER, Houston, TX (US)

(21) Appl. No.: 17/409,504

(22) Filed: Aug. 23, 2021

Related U.S. Application Data
(60) Provisional application No. 63/068,688, filed on Aug. 21, 2020.

(51) Int. Cl. C12N 5/0783; C12N 15/85; C07K 14/725

(52) U.S. Cl. CPC C12N 5/0638; C12N 15/85; C12N 2501/998; C12N 2800/107; C07K 14/7051

(57) ABSTRACT

A method of producing an engineered T cell population includes obtaining a cell population comprising a CD8+ T cell, isolating the CD8+ T cell from the obtained cell population, activating the isolated CD8+ T cell, introducing a nucleic acid encoding a T cell receptor (TCR) binding to an antigen in a complex with an MHC molecule into the activated CD8+ T cell, and expanding the introduced CD8+ T cell to obtain the engineered T cell population.

### CROSS-REFERENCE TO RELATED APPLICATIONS

This application claims priority to U.S. Provisional Patent Application No. 63/068,688, filed 21 Aug. 2020. This application is incorporated by reference in its entirety.

### REFERENCE TO SEQUENCE LISTING SUBMITTED AS A COMPLIANT ASCII TEXT FILE (.txt)

Pursuant to the EFS-Web legal framework and 37 CFR §§ 1.821-825 (see MPEP § 2442.03(a)), a Sequence Listing in the form of an ASCII-compliant text file (entitled “3000011-018001_Sequence_Listing_ST25.txt” created on 20 Aug. 2021, and 227,656 bytes in size) is submitted concurrently with the instant application, and the entire contents of the Sequence Listing are incorporated herein by reference.

### FIELD

The present disclosure generally relates to methods of manufacturing T cells for adoptive immunotherapy. The disclosure further provides for methods of genetically transducing T cells, methods of using T cells, and T cell populations thereof.

### BACKGROUND

In adoptive cell therapy, lymphocytes isolated from a patient may be genetically modified ex vivo to express recombinant proteins that enable the cells to perform new therapeutic functions after subsequently transfer back into the patient. For example, T cells may be isolated from the lymphocytes and genetically modified to express a recombinant chimeric antigen receptor (“CAR T cells”) and/or a T-cell receptor (“TCR therapy”). In CAR T cell therapy, the cells recognize antigens expressed on the surface of cells, whereas TCR therapy cells recognize tumor-specific proteins inside the cells, presented on the surface in an MHC complex. TCR cells are generally engineered to recognize a tumor-specific antigen/MHC combination.

Before the modified T cells are transferred back into the patient, the modified T cells are expanded ex vivo to create a sufficient number of cells to achieve a therapeutic effect. When lymphocytes isolated and returned to the same patient it is generally referred to as “autologous cell therapy”. When the lymphocytes are isolated from a compatible donor and infused into a new, different patient, the process is generally referred to as “allogenic cell therapy.”

The pool of lymphocytes, from which CD8+ T cells for adoptive immunotherapy can be derived, may contain naive and long-lived antigen experienced memory T cells (T_{M}). T_{M} can be divided further into subsets of central memory (T_{CM}) and effector memory (T_{EM}) cells that differ in phenotype, homing properties and functions. CD8+T_{CM} express CD62L and CCR7, which promote migration into lymph nodes, and proliferate rapidly if re-exposed to antigen. CD8+T_{EM} lack CD62L enabling migration to peripheral tissues and exhibit immediate effector function. In response to antigen stimulation, CD8+T_{CM} and T_{EM} both differentiate into cytolytic effector T cells (T_{E}) that express a high level of granzymes and perforin but are short-lived. Thus, the poor survival of T cells in clinical immunotherapy trials may simply result from their differentiation during in vitro culture to T_{E} that are destined to die.

There is a need in the art for a rapid, streamlined, and safe method to isolate lymphocytes, genetically modify and expand the genetically modified lymphocytes ex vivo. Such methods may expand the deployment of methods of adoptive cell therapy, such as chimeric antigen receptor technologies (CAR-T) and T cell receptor technologies (TCR-T), which may hold promise for many patients who currently are in need of an effective cancer treatment.

### SUMMARY OF VARIOUS EMBODIMENTS

The present disclosure relates to methods of methods for producing a CD8+ cytotoxic T lymphocyte (CTL) comprising (a) isolating CD8+ T cells from peripheral blood mononuclear cells (PBMC), (b) activating the isolated CD8+ T cells with an anti-CD3 antibody and an anti-CD28 antibody, (c) introducing a nucleic acid into the activated CD8+ T cells, (d) expanding the transformed CD8+ T cells, and (e) harvesting the transformed CD8+ T cells, wherein step (a) through the step (e) are performed within 6 days. In another aspect, the method takes no longer than 6 days to complete. In an aspect, the method may take 1, 2, 3, 4, 5, 6, 7, 10 or 14 days to complete. The method may further comprise cryopreserving the harvested T-cells. In another aspect, the total time to complete steps (b), (c), (d) and (e) may be from about 6 days to about to about 10 days. In another aspect, activation (b) may be carried out within a period of from about 15 hours to about 24 hours, transduction (c) may be carried out from about 20 hours to about 28 hours, and expansion (d) may be carried out from about 5 days to about 6 days.

In an embodiment, the peripheral blood mononuclear cells (PBMC) may be obtained from a healthy donor. The peripheral blood mononuclear cells (PBMC) may be obtained from a patient. The peripheral blood mononuclear cells (PBMC) may be autologous.

In an embodiment, the number of the isolated CD8+ T cells may be from about 1×10⁸ to about 3×10⁹, from about 2×10⁸ to about 3×10⁹, from about 3×10⁸ to about 3×10⁹, from about 4×10⁸ to about 3×10⁹, from about 5×10⁸ to about 3×10⁹, from about 6×10⁸ to about 3×10⁹, from about 7×10⁸ to about 3×10⁹, from about 8×10⁸ to about 3×10⁹, from about 9×10⁸ to about 3×10⁹, from about 1×10⁹ to about 3×10⁹, from about 1×10⁹ to about 2.5×10⁹, from about 1×10⁹ to about 2×10⁹, or from about 1×10⁹ to about 1.5×10⁹. The number of the isolated CD8+ T cells may be about 1×10⁸ cells, 2×10⁸ cells, 3×10⁸ cells, 4×10⁸ cells, 5×10⁸ cells, 6×10⁸ cells, 7×10⁸ cells, 8×10⁸ cells, 9×10⁸ cells, 1×10⁹ cells, 2×10⁹ cells, 3×10⁹ cells, 4×10⁹ cells, 5×10⁹ cells, 6×10⁹ cells, 7×10⁹ cells, 8×10⁹ cells, 9×10⁹ cells, or 1×10¹⁰ cells.

In an embodiment, the purity of the isolated CD8+ T cells in a preparation may be from about 60% to about 100%, from about 65% to about 100%, from about 70% to about 100%, from about 75% to about 100%, from about 80% to about 100%, from about 85% to about 100%, from about 90% to about 100%, from about 95% to about 100%, from about 96% to about 100%, from about 97% to about 100%, from about 98% to about 100%, or from about 99% to about 100%. The purity of the isolated CD8+ T cells in a preparation may be about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In an embodiment, the CD8+ T cells are CD4+.

In an embodiment, the anti-CD3 antibody may be in a concentration of from about 0.1 μg/ml to about 10.0 μg/ml, about 0.1 μg/ml to about 8.0 μg/ml, about 0.1 μg/ml to about 6.0 μg/ml, about 0.1 μg/ml to about 4.0 μg/ml, about 0.1 μg/ml to about 2.0 μg/ml, about 0.1 μg/ml to about 1.0 μg/ml, about 0.1 μg/ml to about 0.8 μg/ml, about 0.1 μg/ml to about 0.6 μg/ml, about 0.1 μg/ml to about 0.5 μg/ml, about 0.1 μg/ml to about 0.25 μg/ml, about 0.2 μg/ml to about 0.5 μg/ml, about 0.2 μg/ml to about 0.3 μg/ml, about 0.3 μg/ml to about 0.5 μg/ml, about 0.3 μg/ml to about 0.4 μg/ml, or about 0.4 μg/ml to about 0.5 μg/ml. The anti-CD3 antibody may be in a concentration of about 0.1, 0.2, 0.3, 0.4, 0.5, 0.6, 0.7, 0.8, 0.9, 1, 2, 3, 4, 5, 6, 7, 8, 9, or 10 μg/ml.

In an embodiment, the anti-CD28 antibody may be in a concentration of from about 0.1 μg/ml to about 10.0 μg/ml, about 0.1 μg/ml to about 8.0 μg/ml, about 0.1 μg/ml to about 6.0 μg/ml, about 0.1 μg/ml to about 4.0 μg/ml, about 0.1 μg/ml to about 2.0 μg/ml, about 0.1 μg/ml to about 1.0 μg/ml, about 0.1 μg/ml to about 0.8 μg/ml, about 0.1 μg/ml to about 0.6 μg/ml, about 0.1 μg/ml to about 0.5 μg/ml, about 0.1 μg/ml to about 0.25 μg/ml, about 0.2 μg/ml to about 0.5 μg/ml, about 0.2 μg/ml to about 0.3 μg/ml, about 0.3 μg/ml to about 0.5 μg/ml, about 0.3 μg/ml to about 0.4 μg/ml, or about 0.4 μg/ml to about 0.5 μg/ml. The anti-CD28 antibody may be in a concentration of about 0.1, 0.2, 0.3, 0.4, 0.5, 0.6, 0.7, 0.8, 0.9, 1, 2, 3, 4, 5, 6, 7, 8, 9, or 10 μg/ml.

In any embodiment, both the anti-CD3 antibody and the anti-CD28 antibody may each be in a concentration of from about 0.1 μg/ml to about 10.0 μg/ml, about 0.1 μg/ml to about 8.0 μg/ml, about 0.1 μg/ml to about 6.0 μg/ml, about 0.1 μg/ml to about 4.0 μg/ml, about 0.1 μg/ml to about 2.0 μg/ml, about 0.1 μg/ml to about 1.0 μg/ml, about 0.1 μg/ml to about 0.8 μg/ml, about 0.1 μg/ml to about 0.6 μg/ml, about 0.1 μg/ml to about 0.5 μg/ml, about 0.1 μg/ml to about 0.25 μg/ml, about 0.2 μg/ml to about 0.5 μg/ml, about 0.2 μg/ml to about 0.3 μg/ml, about 0.3 μg/ml to about 0.5 μg/ml, about 0.3 μg/ml to about 0.4 μg/ml, or about 0.4 μg/ml to about 0.5 μg/ml. The both the anti-CD3 antibody and the anti-CD28 antibody may be in a concentration of about 0.1, 0.2, 0.3, 0.4, 0.5, 0.6, 0.7, 0.8, 0.9, 1, 2, 3, 4, 5, 6, 7, 8, 9, or 10 μg/ml. In one embodiment, the concentration of the combination of the anti-CD3 antibody and the anti-CD28 antibody may be in a concentration of about 0.1, 0.2, 0.3, 0.4, 0.5, 0.6, 0.7, 0.8, 0.9, 1, 2, 3, 4, 5, 6, 7, 8, 9, or 10 μg/ml.

In an embodiment, the activation of the CD8+ T cells may be completed within a period of about 1 hour to about 120 hours, about 1 hour to about 108 hours, about 1 hour to about 96 hours, about 1 hour to about 84 hours, about 1 hour to about 72 hours, about 1 hour to about 60 hours, about 1 hour to about 48 hours, about 1 hour to about 36 hours, about 1 hour to about 24 hours, about 2 hours to about 24 hours, about 4 hours to about 24 hours, about 6 hours to about 24 hours, about 16 hours to about 20 hours, about 8 hours to about 24 hours, about 10 hours to about 24 hours, about 12 hours to about 24 hours, about 12 hours to about 72 hours, about 24 hours to about 72 hours, about 6 hours to about 48 hours, about 24 hours to about 48 hours, about 6 hours to about 72 hours, or about 1 hour to about 12 hours. The activation of the CD8+ T cells may be completed in about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours. The activation of the CD8+ T cells may be carried for about 1-10 hours, 11-30 hours, 31-50 hours, 51-100 hours, or 101-120 hours. The activation of the CD8+ T cells may be completed in about 1, 2, 3, 4, 5, 6, 7, 8, 9, 10, 11, 12, 13, 14, 15, 16, 17, 18, 19, or 20 days. The activation of the CD8+ T cells may be completed in about 1-14 days. The activation of the CD8+ T cells may be completed in about 13 days.

In an embodiment, the anti-CD3 antibody, the anti-CD28 antibody, or both may be immobilized on a solid support. The solid support may be in the form of a bead, box, column, cylinder, disc, dish (e.g., glass dish, PETRI dish), fibre, film, filter, microtiter plate (e.g., 96-well microtiter plate), multi-bladed stick, net, pellet, plate, ring, rod, roll, sheet, slide, stick, tray, tube, or vial. The solid phase support can be a singular discrete body (e.g., a single tube, a single bead), any number of a plurality of substrate bodies (e.g., a rack of 10 tubes, several beads), or combinations thereof (e.g., a tray comprises a plurality of microtiter plates, a column filled with beads, a microtiter plate filed with beads). The solid support may be a surface of a bead, tube, tank, tray, dish, a plate, a flask, or a bag. The solid support may be an array. The solid support may be a bag.

In an embodiment, the introduction of a nucleic acid into the T cell may comprise transfecting a naked DNA comprising the nucleic acid. The introduction of a nucleic acid into the T cell may comprise transducing a viral vector comprising the nucleic acid. The viral vector may be a retroviral vector, an adenoviral vector, an adeno-associated viral vector, or a lentiviral vector. The nucleic acid may encode a recombinant protein. The recombinant protein may be a chimeric antigen receptor (CAR), a T cell receptor (TCR), a cytokine, an antibody, or a bi-specific binding molecule. The nucleic acid may encode a T cell receptor (TCR).

In one embodiment, the expansion of the T cells may be in the presence of a cytokine. The cytokine may be interferon alpha (IFN-α), interleukin-2 (IL-2), interleukin-4 (IL-4), interleukin-7 (IL-7), interleukin-9 (IL-9), interleukin-12 (IL-12), interleukin-15 (IL-15), interleukin-21 (IL-21), macrophage colony-stimulating factor (MCSF), interleukin-6 (IL-6), eotaxin-1/CCL11, interferon gamma induced protein 10 (IP-10), IL-RA, macrophage inflammatory protein 1 alpha (MIP-1α), macrophage inflammatory protein 1 beta (MIP-1□), interleukin 13 (IL-13), IL-2R, or a combination thereof. The T cells may be expanded in the presence of IL-2.

In one embodiment, the activation of the T cells may be in the presence of a cytokine. The cytokine may be interferon alpha (IFN-α), interleukin-2 (IL-2), interleukin-4 (IL-4), interleukin-7 (IL-7), interleukin-9 (IL-9), interleukin-12 (IL-12), interleukin-15 (IL-15), interleukin-21 (IL-21), macrophage colony-stimulating factor (MCSF), interleukin-6 (IL-6), eotaxin-1/CCL11, interferon gamma induced protein 10 (IP-10), IL-RA, macrophage inflammatory protein 1 alpha (MIP-1□), macrophage inflammatory protein 1 beta (MIP-1□), interleukin 13 (IL-13), IL-2R, or a combination thereof. The T cells may be activated in the presence of IL-2, preferably human IL-2, more preferably recombinant human IL-2 (rhIL-2).

In any embodiment, the cytokine may be interferon alpha (IFN-α), interleukin-2 (IL-2), interleukin-4 (IL-4), interleukin-7 (IL-7), interleukin-9 (IL-9), interleukin-12 (IL-12), interleukin-15 (IL-15), interleukin-21 (IL-21), macrophage colony-stimulating factor (MCSF), interleukin-6 (IL-6), eotaxin-1/CCL11, interferon gamma induced protein 10 (IP-10), IL-RA, macrophage inflammatory protein 1 alpha (MIP-1□), macrophage inflammatory protein 1 beta (MIP-1 □), interleukin 13 (IL-13), IL-2R, or a combination thereof and the cytokine may be present in an amount at about 1 ng/mL and 500 ng/mL. The cytokine may be present in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ng/mL. The cytokine may be present in an amount between about 1 ng/mL and 100 ng/mL, about 100 ng/mL and 200 ng/mL, about 100 ng/mL and 500 ng/mL, about 250 ng/mL and 400 ng/mL, about 10 ng/mL and 100 ng/mL, or about 150 ng/mL and 350 ng/mL.

In one embodiment, the cytokine may comprise a combination of IL-7 and IL-15.

In one embodiment, the concentration of IL-7 may be from about 1 ng/ml to 100 ng/ml, about 1 ng/ml to 90 ng/ml, about 1 ng/ml to 80 ng/ml, about 1 ng/ml to 70 ng/ml, about 1 ng/ml to 60 ng/ml, about 1 ng/ml to 50 ng/ml, about 1 ng/ml to 40 ng/ml, about 1 ng/ml to 30 ng/ml, about 1 ng/ml to 20 ng/ml, about 1 ng/ml to 15 ng/ml, or about 1 ng/ml to 10 ng/ml.

In one embodiment, the IL-7 may be present in an amount at about 1 ng/mL and 500 ng/mL. The cytokine may be present in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ng/mL. The cytokine may be present in an amount between about 1 ng/mL and 100 ng/mL, about 100 ng/mL and 200 ng/mL, about 100 ng/mL and 500 ng/mL, about 250 ng/mL and 400 ng/mL, about 10 ng/mL and 100 ng/mL, or about 150 ng/mL and 350 ng/mL.

In one embodiment, the concentration of IL-15 may be from about 5 ng/ml to 500 ng/ml, about 5 ng/ml to 400 ng/ml, about 5 ng/ml to 300 ng/ml, about 5 ng/ml to 200 ng/ml, about 5 ng/ml to 150 ng/ml, about 5 ng/ml to 100 ng/ml, about 10 ng/ml to 100 ng/ml, about 20 ng/ml to 100 ng/ml, about 30 ng/ml to 100 ng/ml, about 40 ng/ml to 100 ng/ml, or about 50 ng/ml to 100 ng/ml.

In an embodiment, the IL-15 may be present in an amount at about 1 ng/mL and 500 ng/mL. The cytokine may be present in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 ng/mL. The cytokine may be present in an amount between about 1 ng/mL and 100 ng/mL, about 100 ng/mL and 200 ng/mL, about 100 ng/mL and 500 ng/mL, about 250 ng/mL and 400 ng/mL, about 10 ng/mL and 100 ng/mL, or about 150 ng/mL and 350 ng/mL.

In one embodiment, the step (a) through the step (e) may be performed in a closed system.

In an embodiment, the number of the harvested T cells produced by the methods described herein may be from about 1×10⁹ to about 1×10¹³, about 1×10⁹ to about 5×10¹², about 1×10⁹ to about 1×10¹², about 1×10⁹ to about 5×10¹¹, about 1×10⁹ to about 1×10¹¹, about 1×10⁹ to about 5×10¹⁰, about 1×10⁹ to about 1×10¹⁰, about 2×10⁹ to about 1×10¹⁰, about 3×10⁹ to about 1×10¹⁰, about 4×10⁹ to about 1×10¹⁰, about 5×10⁹ to about 1×10¹⁰, about 6×10⁹ to about 1×10¹⁰, about 7×10⁹ to about 1×10¹⁰, about 8×10⁹ to about 1×10¹⁰, or about 9×10⁹ to about 1×10¹⁰ cells.

In an embodiment, the number of the harvested T cells produced by the methods described herein may be about 1×10⁹ cells, 2×10⁹ cells, 3×10⁹ cells, 4×10⁹ cells, 5×10⁹ cells, 6×10⁹ cells, 7×10⁹ cells, 8×10⁹ cells, 9×10⁹ cells, 1×10¹⁰ cells, 1×10¹⁰ cells, 2×10¹⁰ cells, 3×10¹⁰ cells, 4×10¹⁰ cells, 5×10¹⁰ cells, 6×10¹⁰ cells, 7×10¹⁰ cells, 8×10¹⁰ cells, 9×10¹⁰ cells, 1×10¹¹ cells, 2×10¹¹ cells, 3×10¹¹ cells, 4×10¹¹ cells, 5×10¹¹ cells, 6×10¹¹ cells, 7×10¹¹ cells, 8×10¹¹ cells, 9×10¹¹ cells, 1×10¹² cells, 2×10¹² cells, 3×10¹² cells, 4×10¹² cells, 5×10¹² cells, 6×10¹² cells, 7×10¹² cells, 8×10¹² cells, 9×10¹² cells, 1×10¹³ cells, 2×10¹³ cells, 3×10¹³ cells, 4×10¹³ cells, 5×10¹³ cells, 6×10¹³ cells, 7×10¹³ cells, 8×10¹³ cells, 9×10¹³ cells, or 1×10¹⁴ cells.

In one embodiment, a population of genetically modified T cells may be produced by the methods described herein.

In an embodiment, a method of treating a patient who has cancer may comprise administering to the patient a composition comprising a population of genetically modified T cells described herein, wherein the genetically modified T cells kill cancer cells that present a peptide in a complex with an MHC molecule on the surface, wherein the peptide is selected from SEQ ID NO: 1-160, and the cancer is selected from the group consisting of hepatocellular carcinoma (HCC), colorectal carcinoma (CRC), glioblastoma (GB), gastric cancer (GC), esophageal cancer, non-small cell lung cancer (NSCLC), pancreatic cancer (PC), renal cell carcinoma (RCC), benign prostate hyperplasia (BPH), prostate cancer (PCA), ovarian cancer (OC), melanoma, breast cancer, chronic lymphocytic leukemia (CLL), Merkel cell carcinoma (MCC), small cell lung cancer (SCLC), Non-Hodgkin lymphoma (NHL), acute myeloid leukemia (AML), gallbladder cancer and cholangiocarcinoma (GBC, CCC), urinary bladder cancer (UBC), acute lymphocytic leukemia (ALL), uterine cancer (UEC), synovial sarcoma, myxoid liposarcoma, round cell liposarcoma, metastatic rectal mucosal melanoma, urothelial cancer, melanoma, esophagogastric junction (EGJ) cancer, non-small cell lung cancer (NSCLC), head and neck cancer, myxoid/round cell liposarcoma (MRCLS), multiple myeloma, neoplasm, or a combination thereof. The MHC molecule may be MHC Class I.

In one embodiment, the composition may further comprise an adjuvant.

In one embodiment, the adjuvant may be an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, particulate formulations with poly(lactide co-glycolide) (PLG), virosomes, interleukin-1 (IL-1), interleukin-2 (IL-2), interleukin-4 (IL-4), interleukin-7 (IL-7), interleukin-12 (IL-12), interleukin-13 (IL-13), interleukin-15 (IL-15), interleukin-21 (IL-21), interleukin-23 (IL-23), or a combination thereof.

In one embodiment, a method of eliciting an immune response in a patient who has cancer may comprise administering to the patient a composition comprising the population of genetically modified T cells described herein, wherein the genetically modified T cells kill cancer cells that present a peptide in a complex with an MHC molecule on the surface, wherein the peptide is selected from SEQ ID NO: 1-160, wherein the cancer is selected from the group consisting of hepatocellular carcinoma (HCC), colorectal carcinoma (CRC), glioblastoma (GB), gastric cancer (GC), esophageal cancer, non-small cell lung cancer (NSCLC), pancreatic cancer (PC), renal cell carcinoma (RCC), benign prostate hyperplasia (BPH), prostate cancer (PCA), ovarian cancer (OC), melanoma, breast cancer, chronic lymphocytic leukemia (CLL), Merkel cell carcinoma (MCC), small cell lung cancer (SCLC), Non-Hodgkin lymphoma (NHL), acute myeloid leukemia (AML), gallbladder cancer and cholangiocarcinoma (GBC, CCC), urinary bladder cancer (UBC), acute lymphocytic leukemia (ALL), uterine cancer (UEC), synovial sarcoma, myxoid liposarcoma, round cell liposarcoma, metastatic rectal mucosal melanoma, urothelial cancer, melanoma, esophagogastric junction (EGJ) cancer, head and neck cancer, myxoid/round cell liposarcoma (MRCLS), multiple myeloma, neoplasm, or a combination thereof.

In an embodiment, the activating may be performed in a serum free medium.

In another embodiment, the introducing may be performed in a serum free medium.

In another embodiment, the activating and the introducing may be performed in a serum free medium.

In another embodiment, the activating and the introducing may be performed in a serum free medium and the expanding may be performed in the presence of serum.

In an embodiment, the viral vector may be pseudotyped with an envelope protein of vesicular stomatitis virus (VSV-G).

In another embodiment, the activating may be in the presence of a statin.

In another embodiment, the statin may be selected from atorvastatin, cerivastatin, dalvastatin, fluindostatin, fluvastatin, mevastatin, pravastatin, simvastatin, velostatin, and rosuvastatin.

In an embodiment, the methods may further comprise administering a chemotherapy agent. The dosage of the chemotherapy agent may be sufficient to deplete the patient's T-cell population. The chemotherapy may be administered about 4-7 days or about 5 to 7 days prior to T-cell administration. The chemotherapy agent may be cyclophosphamide, fludarabine, or a combination thereof. The chemotherapy agent may comprise dosing at about 400-600 mg/m²/day of cyclophosphamide. The chemotherapy agent may comprise dosing at about 10-30 mg/m²/day of fludarabine.

In an embodiment, the methods may further comprise pre-treatment of the patient low-dose radiation prior to administration of the composition comprising T-cells. The low dose radiation may comprise about 1.4 Gy for 1-6 days, preferably about 5 days, prior to administration of the composition comprising T-cells,

In an embodiment, the patient may be HLA-A*02.

In an embodiment, the patient may be HLA-A*06.

In an embodiment, the methods may further comprise administering an anti-PD1 antibody. The anti-PD1 antibody may be a humanized antibody. The anti-PD1 antibody may be pernbrolizumab. The dosage of the anti-PD1 antibody may be about 200 trig. The anti-PD1 antibody, may be administered every 3 weeks following T-cell administration.

In an embodiment, the dosage of T-cells may be between about 0.8-1.2×10⁹ T cells. The dosage of the T cells may be about 0.5×10⁸ to about 10×10⁹ T cells. The dosage of T-cells may be about 1.2-3×10⁹ T cells, about 3-6×10⁹ T cells, about 10×10⁹ T cells, about 5×10⁹ T cells, about 0.1×10⁹ T cells, about 1×10⁸ T cells, about 5×10⁸ T cells, about 1.2-6×10⁹ T cells, about 1-6×10⁹ T cells, or about 1-8×10⁹ T cells.

In an embodiment, the T cells may be administered in 3 doses. The T-cell doses may escalate with each dose. The T-cells may be administered by intravenous infusion.

In an embodiment, method of producing an engineered T cell population may include obtaining a cell population comprising a CD8+ T cell, isolating the CD8+ T cell from the obtained cell population, activating the isolated CD8+ T cell, introducing a nucleic acid encoding a T cell receptor (TCR) binding to an antigen in a complex with an MHC molecule into the activated CD8+ T cell, and expanding the introduced CD8+ T cell to obtain the engineered T cell population.

In an embodiment, the cell population may contain peripheral blood mononuclear cell (PBMC).

In an embodiment, the PBMC may contain less than 25% of CD8+ cells.

In an embodiment, the isolating may include contacting the CD8+ cell with an anti-CD8 antibody.

In an embodiment, the activating may be performed in the presence of an anti-CD3 antibody and an anti-CD28 antibody.

In an embodiment, the TCR may be selected from Table 1.

In an embodiment, the antigen may be selected from SEQ ID NO: 1-161.

In an embodiment, the TCR may bind to SLLQHLIGL (SEQ ID NO: 50).

In an embodiment, the TCR may be selected from R11KEA (SEQ ID NO: 162 and 163), R11P3D3 (SEQ ID NO: 204 and 205), R16P1C10 (SEQ ID NO: 206 and 207), R16P1E8 (SEQ ID NO: 208 and 209), R17P1A9 (SEQ ID NO: 210 and 211), R17P1D7 (SEQ ID NO: 212 and 213), R17P1G3 (SEQ ID NO: 214 and 215), R17P2B6 (SEQ ID NO: 216 and 217), and R11P3D3KE (SEQ ID NO: 218 and 219).

In an embodiment, the MHC molecule may be a class I MHC molecule.

In an embodiment, a composition may contain an engineered T cell population produced by the method of the present disclosure.

In an embodiment, the composition may further contain at least one adjuvant selected from an anti-CD40 antibody, imiquimod, resiquimod, GM-CSF, cyclophosphamide, sunitinib, bevacizumab, atezolizumab, interferon-alpha, interferon-beta, CpG oligonucleotides and derivatives, poly-(I:C) and derivatives, RNA, sildenafil, particulate formulations with poly(lactide co-glycolide) (PLG), virosomes, interleukin (IL)-1, IL-2, IL-4, IL-7, IL-12, IL-13, IL-15, IL-21, and IL-23.

In an embodiment, method of producing an engineered T cell population may include obtaining a cell population comprising a T cell, resting the obtained cell population, activating the rested cell population, introducing a nucleic acid encoding a T cell receptor (TCR) binding to an antigen in a complex with an MHC molecule into the activated cell population in the absence of serum, and expanding the introduced cell population to obtain the engineered T cell population.

In an embodiment, the resting may be performed for about 2-8 hours, about 2-6 hours, or about 2-8 hours.

In an embodiment, the resting may be performed in the presence of serum.

In an embodiment, the resting may be performed for about 2-8 hours, about 2-6 hours, or about 2-4 hours.

In an embodiment, the activating may be performed in the presence of an anti-CD3 antibody and an anti-CD28 antibody.

In an embodiment, the activating may be performed in the absence of serum.

In an embodiment, method of producing an engineered T cell population may include, obtaining a cell population comprising a CD8+ T cell, isolating the CD8+ T cell from the obtained cell population, activating the isolated CD8+ T cell, introducing a nucleic acid encoding a chimeric antigen receptor (CAR) binding to an antigen into the activated CD8+ T cell, and expanding the introduced CD8+ T cell to obtain the engineered T cell population.

In an embodiment, method of producing an engineered T cell population may include obtaining a cell population comprising a T cell, resting the obtained cell population, activating the rested cell population, introducing a nucleic acid encoding a chimeric antigen receptor (CAR) binding to an antigen into the activated cell population in the absence of serum, and expanding the introduced cell population to obtain the engineered T cell population.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts recoveries of CD8+ selected T cells from healthy donor whole Leukopak® (an enriched leukapheresis product collected from normal peripheral blood). Results are represented as mean±SD, N=5-7, individual dot represents one donor.

FIG. 1B depicts purities of CD8+ selected T cells from healthy donor whole Leukopak® (an enriched leukapheresis product collected from normal peripheral blood). Results are represented as mean±SD, N=5-7, individual dot represents one donor.

FIG. 2A depicts viability of CD8+ T cells from bulk peripheral blood mononuclear (PBMC) or CD8+ selected T cells at post-anti-CD3/CD28 activation. Individual dot represents one donor. Results are represented as mean±SD, n=5-7.

FIG. 2B depicts recoveries of CD8+ T cells from bulk PBMC or CD8+ selected T cells at post-anti-CD3/CD28 activation. Individual dot represents one donor. Results are represented as mean±SD, n=5-7.

FIG. 2C depicts the percentage of live lymphocytes, % CD3+ cells, % CD8+ cells, and % CD4+ cells at pre- and post-anti-CD3/CD28 activation. Individual dot represents one donor. Results are represented as mean±SD, n=5-7.

FIG. 2D depicts % CD8+ T cells positive for CD69, CD25, and h-LDLR from bulk PBMC or CD8+ selected T cells at pre- and post-anti-CD3/CD28 activation. Individual dot represents one donor. Results are represented as mean±SD, n=5-7.

FIG. 3 depicts memory T cell subset distribution among CD8+ T cells from bulk PBMC and CD8+ selected T cells obtained from donors (Donor Nos. 1-4) at pre- and post-anti-CD3/CD28 activation. Memory phenotype on CD3+CD8+ gated cells based on CD45RA+ and CCR7+ expression: Tₙₐᵢᵥₑ; CD45RA+CCR7+; T_{cm}; CD45RA-CCR7; T_{cm}; CD45RA-CCR7-Tₑₘᵣₐ; CD45RA+CCR7-.

FIGS. 4A-41 depicts cytokine secretion profile IFNγ (FIG. 4A), TNF-α (FIG. 4B), perforin (FIG. 4C), IL-1β (FIG. 4D), RANTES (FIG. 4E), MIP-1a (FIG. 4F), IL-13 (FIG. 4G), IL-17A (FIG. 4H), and IL-10 (FIG. 4I), of PBMC and CD8+ selected cells at pre- and post-anti-CD3/CD28 activation. Cytokines secreted by pre- and post-activated PBMC and CD8+ selected cells after 24 hours in culture were quantified using 34-plex Luminex Technology (N=2). Individual dot represents one donor.

FIG. 5A depicts viabilities of PBMC- and CD8-selected T cell products obtained from bulk PBMC and CD8+ selected T cells activated and transduced with LV-TCR (e.g., TCR R11KEA (SEQ ID NOS: 162 and 163) that binds to SLLQHLIGL (PRAME-004) peptide/MHC molecule complex) and expanded in IL-7 and IL-15 for 6 days. Results are represented as mean±SD, N=5-7; t-test, p>0.05 is non-significant.

FIG. 5B depicts fold expansion of PBMC- and CD8-selected T cell products obtained from bulk PBMC and CD8+ selected T cells activated and transduced with LV-TCR and expanded in IL-7 and IL-15 for 6 days. Results are represented as mean±SD, N=5-7.

FIG. 5C depicts absolute cell counts of PBMC- and CD8-selected T cell products obtained from bulk PBMC and CD8+ selected T cells activated and transduced with LV-TCR and expanded in IL-7 and IL-15 for 6 days. Results are represented as mean±SD, N=5-7.

FIG. 5D depicts % Dextramer+ on CD8+ cells of PBMC- and CD8-selected T cell products obtained from bulk PBMC and CD8+ selected T cells activated and transduced with LV-TCR and expanded in IL-7 and IL-15 for 6 days. Results are represented as mean±SD, N=5-7; t-test, p>0.05 is non-significant.

FIG. 5E depicts the vector copy number in PBMC- and CD8-selected T cell products obtained from bulk PBMC and CD8+ selected T cells activated and transduced with LV-TCR and expanded in IL-7 and IL-15 for 6 days. Results are represented as mean±SD, N=5-7; t-test, p>0.05 is non-significant.

FIG. 6A depicts the memory T cell distribution in CD3+CD8+Dex+ gated cells from PBMC LV-TCR or CD8 LV-TCR products based on CD45RA+ and CCR7+ expression: Tₙₐᵢᵥₑ; CD45RA+CCR7+; Tₑₘ; CD45RA-CCR7; Tₑₘ; CD45RA-CCR7-Tₑₘᵣₐ; CD45RA+CCR7-. Results are represented as mean±SD, N=5-7. Individual dot represents one donor.

FIG. 6B depicts the MFI (mean fluorescence intensity) of CD27, CD28, and CD127 on CD3+CD8+Dex+ cells from PBMC LV-TCR or CD8 LV-TCR products. Results are represented as mean±SD, N=5-7. Individual dot represents one donor.

FIG. 6C depicts the expression of exhaustion markers on CD3+CD8+Dex+ cells from PBMC LV-TCR or CD8 LV-TCR products. Non-transduced cells (PBMC NT and CD8 NT) serve as controls. Results are represented as mean±SD, N=5-7. Individual dot represents one donor.

FIG. 7 depicts the total CD3+CD8+Dex+ cell counts in PBMC LV-TCR and CD8 LV-TCR products tested at mid-scale, large-scale and the GMP-scale. Individual dot represents one donor. Results are represented as mean±SD, N=5-7 at different scales.

FIG. 8 shows intracellular cytokine secretion by LV-TCR products derived from bulk PBMC and CD8+ T cells in response to HLA-A*02+ target cell line. PBMC- or CD8-derived LV-TCR product was cocultured with PRAME+ UACC257 (1080 copies) cell line at 1:1::E:T ratio for 12 hours and intracellularly stained for Granzyme B, IFN-γ, TNF-α, MIP-1 and IL-2. Non-transduced cells (PBMC NT and CD8 NT) serve as controls. Results are represented as mean±SD, N=3.

FIG. 9A depicts an IncuCyte killing assay, wherein PBMC LV-TCR or CD8 LV-TCR products (normalized to % CD8+Dex+) were co-cultured with HLA-A*02+ UACC257 cell line (presenting ˜1080 copies of PRAME antigen) at E:T ratio of 4:1 for 24 hours. Tumor growth was assessed using IncuCyte S3. Non-transduced cells (PBMC NT and CD8 NT) serve as controls. Representative donor is shown, N=2.

FIG. 9B depicts an IncuCyte killing assay, wherein PBMC LV-TCR or CD8 LV-TCR products (normalized to % CD8+Dex+) were co-cultured with HLA-A*02+U2-OS cell line (presenting ˜250 copies of PRAME antigen) at E:T ratio of 4:1 for 24 h. Tumor growth was assessed using IncuCyte S3. Non-transduced cells (PBMC NT and CD8 NT) serve as controls. Representative donor is shown, N=2.

FIG. 9C depicts an IncuCyte killing assay, wherein PBMC LV-TCR or CD8 LV-TCR products (normalized to % CD8+Dex+) were co-cultured with HLA-A*02+A375 cell line (presenting ˜50 copies of PRAME antigen) at E:T ratio of 4:1 for 24 h. Tumor growth was assessed using IncuCyte S3. Non-transduced cells (PBMC NT and CD8 NT) serve as controls. Representative donor is shown, N=2.

FIG. 9D depicts an IncuCyte killing assay, wherein PBMC LV-TCR or CD8 LV-TCR products (normalized to % CD8+Dex+) were co-cultured with HLA-A*02+ MCF7 cell line (presenting 0 copies of PRAME antigen) at E:T ratio of 4:1 for 24 h. Tumor growth was assessed using IncuCyte S3. Non-transduced cells (PBMC NT and CD8 NT) serve as controls. Representative donor is shown, N=2.

FIG. 10 depicts the cytokine secretion by PBMC LV-TCR and CD8 LV-TCR products in response to the targets. PBMC LV-TCR and CD8 LV-TCR products (normalized to % CD8+Dex+) was co-cultured with multiple HLA-A*02+ target cell lines expressing varying levels of PRAME antigen; UACC257 (˜1080 copies), U2-OS (˜250 copies), A375 (˜50 copies) and MCF7 (0 copy) at E:T ratio of 4:1 for 24 h. Cytokines released in the culture supernatants were quantified using custom 16-plex Luminex kit (Thermo Fisher). Non-transduced cells (PBMC NT and CD8 NT) serve as controls. Results are represented as mean±SD, N=3.

FIG. 11A depicts the residual tumor cells after PBMC LV-TCR or CD8 LV-TCR products (normalized to % CD8+Dex+) was co-cultured with THP-1 target cell line (PRAME+, ˜80 copies) in complete TexMACS media without cytokines at E:T ratios of 1:1 and 1:5 for 17 days. Results were analyzed by counting beads using flow cytometry. Non-transduced cells serve as controls. Average of 2 donors is shown.

FIG. 11B depicts the residual CD3+ T cells after PBMC LV-TCR or CD8 LV-TCR products (normalized to % CD8+Dex+) was co-cultured with THP-1 target cell line (PRAME+, ˜80 copies) in complete TexMACS media without cytokines at E:T ratios of 1:1 and 1:5 for 17 days. Results were analyzed by counting beads using flow cytometry. Non-transduced cells serve as controls. The average of 2 donors is shown.

FIG. 12 depicts the cytokine secretion by PBMC LV-TCR and CD8 LV-TCR products in response to the THP-1 target cell line. PBMC LV-TCR or CD8 LV-TCR product (normalized to % CD8+Dex+) was co-cultured with THP-1 target cell line (PRAME+, ˜80 copies)) at 1:1 E:T ratios for 24 h. Cytokines released in the culture supernatants were quantified using 34-plex Luminex kit (Thermo Fisher). Non-transduced cells serve as controls. The average of 2 donors is shown.

FIG. 13 depicts the memory T cell distribution in the residual T cells after each round of killing in the PBMC LV-TCR and CD8 LV-TCR products. Memory phenotype on CD3+CD8+Dextramer+ gated cells based on CD45RA+ and CCR7+ expression: Tₙₐᵢᵥₑ; CD45RA+CCR7+; T_{cm}; CD45RA−CCR7; Tₑₘ; CD45RA−CCR7−; Tₑₘᵣₐ; CD45RA+CCR7− at E:T ratio of 1:1. Representative donor is shown.

FIG. 14 depicts a 6-day process for T cell manufacturing in accordance with one embodiment of the present disclosure.

FIG. 15 depicts the effects of serum on the frequency of CD8+TCR+ T cell products (% of CD8+Dextramer) obtained from two donors (upper and lower panels) in accordance with one embodiment of the present disclosure.

FIG. 16 depicts the effects of serum on the vector copy number in T cell products obtained from two donors (upper and lower panels) in accordance with one embodiment of the present disclosure.

FIG. 17 depicts a 6-day process for T cell manufacturing in accordance with another embodiment of the present disclosure.

FIG. 18 depicts, at post-activation, the effect of serum on % viability (left panel), total viable cells (middle panel), and % recovery of T cell products in accordance with an embodiment of the present disclosure.

FIG. 19 depicts the effect of serum on the expression of activation markers, e.g., human low density lipoprotein (hLDL) (left panel), CD69 (middle panel), and CD25 (right panel), of T cell products in accordance with an embodiment of the present disclosure.

FIG. 20 depicts the effect of serum on the expression of activation marker CD25 (CD25+% parent (upper panel) and CD25+ MFI (lower panel)) of T cell products in accordance with an embodiment of the present disclosure.

FIG. 21 depicts the effect of serum on the expression of activation marker CD69 (CD69+% parent (upper panel) and CD69+ MFI (lower panel)) of T cell products in accordance with an embodiment of the present disclosure.

FIG. 22 depicts the effect of serum on the expression of activation marker hLDL (hLDL+% parent (upper panel) and hLDL+ MFI (lower panel)) of T cell products in accordance with an embodiment of the present disclosure.

FIG. 23 depicts the effect of serum on total cells (upper panel) and viability (lower panel) of T cell products in accordance with an embodiment of the present disclosure.

FIG. 24 depicts the effect of serum on CD8+TCR+(CD8+Dex+% parent (upper panel) and CD8+Dex+ MFI (lower panel)) of T cell products in accordance with an embodiment of the present disclosure.

FIG. 25 depicts the effect of serum on CD8+ cell population in accordance with an embodiment of the present disclosure.

FIG. 26 depicts the effect of serum on the vector copy number (VCN) in T cell products in accordance with an embodiment of the present disclosure.

### DETAILED DESCRIPTION OF PREFERRED EMBODIMENTS

### Adoptive T-Cell Therapy

Adoptive T-cell therapy using genetically modified T cells is an attractive strategy in various clinical settings. Ho et al. Cancer Cell (2003) 3: 431-437, the contents of which is incorporated by reference in its entirety. A short, e.g., 6-day, manufacturing process for producing genetically modified T cell products expressing recombinant proteins, such as chimeric antigen receptors (CARs), T cell receptors (TCRs), cytokines, antibodies, and bi-specific binding molecules, yields products with less differentiated memory phenotype as compared to the longer, e.g., 8-10 day, processes. Although a short manufacturing process may be an asset to the adoptive T-cell therapy, the total cell number of functionally transduced T-cells may be compromised by the short manufacturing process, especially when higher T-cell doses are preferred for infusion in cancer patients. To meet the need for higher doses, various strategies can be used to increase the total yield of functionally transduced cells. These may include scaling-up the whole process, enhancing the transduction efficiency or starting from CD8+ selected T cells as opposed to the bulk PBMC for a CD8 dependent TCR. Although further scale-up of the manufacturing process may be achievable, it may be, however, more expensive, more lengthy, and may impact manufacturing capacity.

To address this issue, the inventors used CD8+ selected T cells as starting material to produce genetically modified T cell products expressing recombinant proteins, e.g., CARs, TCRs, cytokines, antibodies, and bi-specific binding molecules, which yield a greater number of genetically modified T cell products, e.g., CAR- or TCR-transformed T cell products than in large- or GMP-scale that manufactured using PBMC as starting materials, while maintaining comparable functionality of genetically modified T cell products manufactured by either process. This lead to a surprising increase in the yield of desired T cells without expensive scale-up, replication costs, or a lengthy processing time (e.g., greater than 7 days).

“Activation” as used herein refers broadly to the state of a T cell that has been sufficiently stimulated to induce detectable cellular proliferation. Activation can also be associated with induced cytokine production, and detectable effector functions. The term “activated T cells” refers to, among other things, T cells that are proliferating.

“Antibodies” and “immunoglobulin” as used herein refer broadly to antibodies or immunoglobulins of any isotype, fragments of antibodies, which retain specific binding to antigen, including, but not limited to, Fab, Fab′, Fab′-SH, (Fab′)₂ Fv, scFv, divalent scFv, and Fd fragments, chimeric antibodies, humanized antibodies, single-chain antibodies, and fusion proteins including an antigen-specific targeting region of an antibody and a non-antibody protein.

“Bispecific binding molecule” and “bispecific antigen binding molecule,” as used herein refer broadly to antigen-binding proteins are able of binding to two different antigens simultaneously, e.g., bispecific antibodies. For example, unlike conventional antibodies, the bispecific antigen binding molecule of the present disclosure may comprise at least 6 CDRs from a TCR. In an embodiment, the antigen binding proteins of the present disclosure, unlike conventional antibodies, may comprise at least one variable alpha domain and at least one variable beta domain from a TCR.

“Chimeric antigen receptor” or “CAR” or “CARs” as used herein refers broadly to genetically modified receptors, which graft an antigen specificity onto cells, for example T cells, NK cells, macrophages, and stem cells. CARs can include at least one antigen-specific targeting region (ASTR), a hinge or stalk domain, a transmembrane domain (TM), one or more co-stimulatory domains (CSDs), and an intracellular activating domain (IAD). In certain embodiments, the CSD is optional. In another embodiment, the CAR is a bispecific CAR, which is specific to two different antigens or epitopes. After the ASTR binds specifically to a target antigen, the IAD activates intracellular signaling. For example, the IAD can redirect T cell specificity and reactivity toward a selected target in a non-MHC-restricted manner, exploiting the antigen-binding properties of antibodies. The non-MHC-restricted antigen recognition gives T cells expressing the CAR the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains.

T cell manufacturing methods disclosed herein may include modifying T cells to express one or more CARs. T cells may be αβ T cells, γδ T cells, or natural killer T cells. In various embodiments, the present disclosure provides T cells genetically engineered with vectors designed to express CARs that redirect cytotoxicity toward tumor cells. CARs are molecules that combine antibody-based specificity for a target antigen, e.g., tumor antigen, with a T cell receptor-activating intracellular domain to generate a chimeric protein that exhibits a specific anti-tumor cellular immune activity. As used herein, the term, “chimeric,” describes being composed of parts of different proteins or DNAs from different origins.

CARs may contain an extracellular domain that binds to a specific target antigen (also referred to as a binding domain or antigen-specific binding domain), a transmembrane domain and an intracellular signaling domain. The main characteristic of CARs may be their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific coreceptors.

In particular embodiments, CARs may contain an extracellular binding domain including but not limited to an antibody or antigen binding fragment thereof, a tethered ligand, or the extracellular domain of a coreceptor, that specifically binds a target antigen that is a tumor-associated antigen (TAA) or a tumor-specific antigen (TSA). In certain embodiments, the TAA or TSA may be expressed on a blood cancer cell. In another embodiment, the TAA or TSA may be expressed on a cell of a solid tumor. In particular embodiments, the solid tumor may be a glioblastoma, a non-small cell lung cancer, a lung cancer other than a non-small cell lung cancer, breast cancer, prostate cancer, pancreatic cancer, liver cancer, colon cancer, stomach cancer, a cancer of the spleen, skin cancer, a brain cancer other than a glioblastoma, a kidney cancer, a thyroid cancer, or the like.

In particular embodiments, the TAA or TSA may be selected from the group consisting of alpha folate receptor, 5T4, αvβ6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRa, GD2, GD3, *Glypican-3 (GPC3), HLA-A1+ MAGE1, HLA-A2+ MAGE1, HLA-A3+ MAGE1, HLA-A1+NY-ESO-1, HLA-A2+NY-ESO-1 HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, and VEGFR2.

In an aspect, T cells expressing CARs can selectively recognize cells, which present a TAA peptide described in SEQ ID NO: 1-161.

Binding Domains of CARs

In particular embodiments, CARs contemplated herein comprise an extracellular binding domain that specifically binds to a target polypeptide, e.g., target antigen, expressed on tumor cell. As used herein, the terms, “binding domain,” “extracellular domain,”

“extracellular binding domain,” “antigen-specific binding domain,” and “extracellular antigen specific binding domain,” may be used interchangeably and provide a CAR with the ability to specifically bind to the target antigen of interest. A binding domain may include any protein, polypeptide, oligopeptide, or peptide that possesses the ability to specifically recognize and bind to a biological molecule (e.g., a cell surface receptor or tumor protein, lipid, polysaccharide, or other cell surface target molecule, or component thereof). A binding domain may include any naturally occurring, synthetic, semi-synthetic, or recombinantly produced binding partner for a biological molecule of interest.

In particular embodiments, the extracellular binding domain of a CAR may include an antibody or antigen binding fragment thereof. An “antibody” refers to a binding agent that is a polypeptide containing at least a light chain or heavy chain immunoglobulin variable region, which specifically recognizes and binds an epitope of a target antigen, such as a peptide, lipid, polysaccharide, or nucleic acid containing an antigenic determinant, such as those recognized by an immune cell. Antibodies may include antigen binding fragments thereof. The term may also include genetically engineered forms, such as chimeric antibodies (for example, humanized murine antibodies), hetero-conjugate antibodies, e.g., bispecific antibodies, and antigen binding fragments thereof. See also, Pierce Catalog and Handbook, 1994-1995 (Pierce Chemical Co., Rockford, Ill.); Kuby, J., Immunology, 3rd Ed., W. H. Freeman & Co., New York, 1997.

In particular embodiments, the target antigen may be an epitope of an alpha folate receptor, 5T4, αvβ6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRa, GD2, GD3, *Glypican-3 (GPC3), HLA-A1+ MAGE1, HLA-A2+ MAGE1, HLA-A3+ MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2 polypeptide.

Light and heavy chain variable regions may contain a “framework” region interrupted by three hypervariable regions, also called “complementarity-determining regions” or “CDRs.” The CDRs can be defined or identified by conventional methods, such as by sequence according to Kabat et al (Wu, TT and Kabat, E. A., J Exp Med. 132(2):211-50, (1970); Borden, P. and Kabat E. A., PNAS, 84: 2440-2443 (1987); (see, Kabat et al, Sequences of Proteins of Immunological Interest, U.S. Department of Health and Human Services, 1991, which is hereby incorporated by reference), or by structure according to Chothia et al (Choithia, C. and Lesk, A. M., J Mol. Biol, 196(4): 901-917 (1987), Choithia, C. et al, Nature, 342: 877-883 (1989)). The contents of the afore-mentioned references are hereby incorporated by reference in their entireties. The sequences of the framework regions of different light or heavy chains may be relatively conserved within a species, such as humans. The framework region of an antibody that is the combined framework regions of the constituent light and heavy chains may serve to position and align the CDRs in three-dimensional space. The CDRs may be primarily responsible for binding to an epitope of an antigen. The CDRs of each chain may be typically referred to as CDR1, CDR2, and CDR3, numbered sequentially starting from the N-terminus, and may be also typically identified by the chain, in which the particular CDR is located. Thus, the CDRs located in the variable domain of the heavy chain of the antibody may be referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) may have different CDRs. Although it is the CDRs that vary from antibody to antibody, only a limited number of amino acid positions within the CDRs are directly involved in antigen binding. These positions within the CDRs are called specificity determining residues (SDRs).

References to “VH” or “VH” refers to the variable region of an immunoglobulin heavy chain, including that of an antibody, Fv, scFv, dsFv, Fab, or other antibody fragment. References to “VL” or “VL” refers to the variable region of an immunoglobulin light chain, including that of an antibody, Fv, scFv, dsFv, Fab, or other antibody fragment.

A “monoclonal antibody” is an antibody produced by a single clone of B lymphocytes or by a cell into which the light and heavy chain genes of a single antibody have been transfected. Monoclonal antibodies may be produced by methods known to those of skill in the art, for example, by making hybrid antibody-forming cells from a fusion of myeloma cells with immune spleen cells. Monoclonal antibodies may include humanized monoclonal antibodies.

A “chimeric antibody” has framework residues from one species, such as human, and CDRs (which generally confer antigen binding) from another species, such as a mouse. In particular preferred embodiments, a CAR disclosed herein may contain antigen-specific binding domain that is a chimeric antibody or antigen binding fragment thereof.

In certain embodiments, the antibody may be a humanized antibody (such as a humanized monoclonal antibody) that specifically binds to a surface protein on a tumor cell. A “humanized” antibody is an immunoglobulin including a human framework region and one or more CDRs from a non-human (for example a mouse, rat, or synthetic) immunoglobulin. Humanized antibodies can be constructed by means of genetic engineering (see for example, U.S. Pat. No. 5,585,089, the content of which is hereby incorporated by reference in its entirety).

In embodiments, the extracellular binding domain of a CAR may contain an antibody or antigen binding fragment thereof, including but not limited to a Camel Ig (a camelid antibody (VHH)), Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, Nanobody).

“Camel Ig” or “camelid VHH” as used herein refers to the smallest known antigen-binding unit of a heavy chain antibody (Koch-Nolte, et al, FASEB J., 21:3490-3498 (2007), the content of which is hereby incorporated by reference in its entirety). A “heavy chain antibody” or a “camelid antibody” refers to an antibody that contains two VH domains and no light chains (Riechmann L. et al, J. Immunol. Methods 231:25-38 (1999); WO94/04678; WO94/25591; U.S. Pat. No. 6,005,079; the contents of which are hereby incorporated by reference in its entirety).

“IgNAR” of “immunoglobulin new antigen receptor” refers to class of antibodies from the shark immune repertoire that consist of homodimers of one variable new antigen receptor (VNAR) domain and five constant new antigen receptor (CNAR) domains.

Papain digestion of antibodies produces two identical antigen-binding fragments, called “Fab” fragments, each with a single antigen-binding site, and a residual “Fc” fragment, whose name reflects its ability to crystallize readily. The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” is the minimum antibody fragment which contains a complete antigen-binding site. In a single-chain Fv (scFv) species, one heavy- and one light-chain variable domain can be covalently linked by a flexible peptide linker such that the light and heavy chains can associate in a “dimeric” structure analogous to that in a two-chain Fv species.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al, Nat. Med. 9:129-134 (2003); and Hollinger et al, PNAS USA 90: 6444-6448 (1993). Triabodies and tetrabodies are also described in Hudson et al, Nat. Med. 9:129-134 (2003). The contents of the afore-mentioned references are hereby incorporated by reference in their entireties.

“Single domain antibody” or “sdAb” or “nanobody” refers to an antibody fragment that consists of the variable region of an antibody heavy chain (VH domain) or the variable region of an antibody light chain (VL domain) (Holt, L., et al, Trends in Biotechnology, 21(11): 484-490, the content of which is hereby incorporated by reference in its entirety).

“Single-chain Fv” or “scFv” antibody fragments comprise the VH and VL domains of antibody, wherein these domains are present in a single polypeptide chain and in either orientation {e.g., VL-VH or VH-VL). Generally, the scFv polypeptide further comprises a polypeptide linker between the VH and VL domains which enables the scFv to form the desired structure for antigen binding.

In a certain embodiment, the scFv binds an alpha folate receptor, 5T4, αvβ6 integrin, BCMA, B7-H3, B7-H6, CALX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRa, GD2, GD3, *Glypican-3 (GPC3), HLA-A1+ MAGE1, HLA-A2+ MAGE1, HLA-A3+ MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2 polypeptide.

Linkers of CARs

In certain embodiments, the CARs may contain linker residues between the various domains, e.g., between VH and VL domains, added for appropriate spacing and conformation of the molecule. CARs may contain one, two, three, four, or five or more linkers. In particular embodiments, the length of a linker may be about 1 to about 25 amino acids, about 5 to about 20 amino acids, or about 10 to about 20 amino acids, or any intervening length of amino acids. In some embodiments, the linker may be 1, 2, 3, 4, 5, 6, 7, 8, 9, 10, 11, 12, 13, 14, 15, 16, 17, 18, 19, 20, 21, 22, 23, 24, 25, or more amino acids long. Illustrative examples of linkers include glycine polymers (G)n; glycine-serine polymers (Gi_sSi_5)n, where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains of fusion proteins, such as CARs. Glycine may access significantly more phi-psi space than even alanine, and may be much less restricted than residues with longer side chains see, Tang et al, Pharmaceutics 2021, 13, 422. the content of which is hereby incorporated by reference in its entirety). The ordinarily skilled artisan may recognize that design of a CAR in particular embodiments can include linkers that may be all or partially flexible, such that the linker can include a flexible linker as well as one or more portions that confer less flexible structure to provide for a desired CAR structure.

In particular embodiments a CAR may include a scFV that may further contain a variable region linking sequence. A “variable region linking sequence,” is an amino acid sequence that connects a heavy chain variable region to a light chain variable region and provides a spacer function compatible with interaction of the two sub-binding domains so that the resulting polypeptide retains a specific binding affinity to the same target molecule as an antibody that may contain the same light and heavy chain variable regions. In one embodiment, the variable region linking sequence may be 1, 2, 3, 4, 5, 6, 7, 8, 9, 10, 11, 12, 13, 14, 15, 16, 17, 18, 19, 20, 21, 22, 23, 24, 25, or more amino acids long. In a particular embodiment, the variable region linking sequence may contain a glycine-serine polymer (Gi_sSi_5)n, where n is an integer of at least 1, 2, 3, 4, or 5. In another embodiment, the variable region linking sequence comprises a (G4S)3 amino acid linker.

Spacer Domains of CARs

In particular embodiments, the binding domain of the CAR may be followed by one or more “spacer domains,” which refers to the region that moves the antigen binding domain away from the effector cell surface to enable proper cell/cell contact, antigen binding and activation (Patel et al, Gene Therapy, 1999; 6: 412-419, the content of which is hereby incorporated by reference in its entirety). The spacer domain may be derived either from a natural, synthetic, semi-synthetic, or recombinant source. In certain embodiments, a spacer domain may be a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region. In one embodiment, the spacer domain may include the CH2 and CH3 of IgG1.

Hinge Domains of CARs

The binding domain of CAR may be generally followed by one or more “hinge domains,” which may play a role in positioning the antigen binding domain away from the effector cell surface to enable proper cell/cell contact, antigen binding and activation. CAR generally may include one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs may include the hinge region derived from the extracellular regions of type 1 membrane proteins, such as CD8a, CD4, CD28 and CD7, which may be wild-type hinge regions from these molecules or may be altered. In another embodiment, the hinge domain may include a CD8a hinge region.

Transmembrane (TM) Domains of CARs

The “transmembrane domain” may be the portion of CAR that can fuse the extracellular binding portion and intracellular signaling domain and anchors CAR to the plasma membrane of the immune effector cell. The TM domain may be derived either from a natural, synthetic, semi-synthetic, or recombinant source. Illustrative TM domains may be derived from (including at least the transmembrane region(s) of) the α, β, or ζ chain of the T-cell receptor, CD3ε, CD3ζ; CD4, CD5, CD9, CD16, CD22, CD27, CD28, CD33, CD37, CD45, CD64, CD80, CD86, CD 134, CD137, and CD154. In one embodiment, CARs may contain a TM domain derived from CD8a. In another embodiment, a CAR contemplated herein comprises a TM domain derived from CD8α and a short oligo- or polypeptide linker, preferably between 1, 2, 3, 4, 5, 6, 7, 8, 9, or 10 amino acids in length that links the TM domain and the intracellular signaling domain of CAR. A glycine-serine linker provides a particularly suitable linker.

Intracellular Signaling Domains of CARs

In particular embodiments, CARs may contain an intracellular signaling domain. An “intracellular signaling domain,” refers to the part of a CAR that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

The term “effector function” refers to a specialized function of the cell. Effector function of the T cell, for example, may be cytolytic activity or help or activity including the secretion of a cytokine. Thus, the term “intracellular signaling domain” refers to the portion of a protein, which can transduce the effector function signal and that direct the cell to perform a specialized function. While usually the entire intracellular signaling domain can be employed, in many cases it is not necessary to use the entire domain. To the extent that a truncated portion of an intracellular signaling domain may be used, such truncated portion may be used in place of the entire domain as long as it can transduce the effector function signal. The term intracellular signaling domain may be meant to include any truncated portion of the intracellular signaling domain sufficient to transducing effector function signal.

It is known that signals generated through TCR alone are insufficient for full activation of the T cell and that a secondary or costimulatory signal may be also required. Thus, T cell activation can be said to be mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. In preferred embodiments, CAR may include an intracellular signaling domain that may contain one or more “costimulatory signaling domain” and a “primary signaling domain.” Primary signaling domains can regulate primary activation of the TCR complex either in a stimulatory way, or in an inhibitory way. Primary signaling domains that act in a stimulatory manner may contain signaling motifs, which are known as immunoreceptor tyrosine-based activation motifs or ITAMs. Illustrative examples of ITAM containing primary signaling domains that are of particular use in the invention may include those derived from TCRζ, FcRγ, FcRβ, CD3γ, CD3δ, CD3ε, CD3ζ CD22, CD79a, CD79b, and CD66d. In particular preferred embodiments, CAR may include a CD3ζ primary signaling domain and one or more costimulatory signaling domains. The intracellular primary signaling and costimulatory signaling domains may be linked in any order in tandem to the carboxyl terminus of the transmembrane domain.

CARs may contain one or more costimulatory signaling domains to enhance the efficacy and expansion of T cells expressing CAR receptors. As used herein, the term, “costimulatory signaling domain,” or “costimulatory domain”, refers to an intracellular signaling domain of a costimulatory molecule. Illustrative examples of such costimulatory molecules may include CD27, CD28, 4-1BB (CD137), OX40 (CD134), CD30, CD40, PD-1, ICOS (CD278), CTLA4, LFA-1, CD2, CD7, LIGHT, TRIM, LCK3, SLAM, DAP10, LAGS, HVEM and NKD2C, and CD83. In one embodiment, CAR may contain one or more costimulatory signaling domains selected from the group consisting of CD28, CD137, and CD134, and a CD3ζ primary signaling domain.

In one embodiment, CAR may contain an scFv that binds an alpha folate receptor, 5T4, αvβ6 integrin, BCMA, B7-H3, B7-H6, CALX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, *Glypican-3 (GPC3), HLA-A1+ MAGE1, HLA-A2+M AGE1, HLA-A3+ MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2 polypeptide; a transmembrane domain derived from a polypeptide selected from the group consisting of: CD8a; CD4, CD45, PD1, and CD152; and one or more intracellular costimulatory signaling domains selected from the group consisting of: CD28, CD54, CD134, CD137, CD152, CD273, CD274, and CD278; and a CD3ζ primary signaling domain.

In another embodiment, CAR may contain an scFv that binds an alpha folate receptor, 5T4, αvβ6 integrin, BCMA, B7-H3, B7-H6, CALX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, *Glypican-3 (GPC3), HLA-A1+ MAGE1, HLA-A2+ MAGE1, HLA-A3+ MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2 polypeptide; a hinge domain selected from the group consisting of: IgG1 hinge/CH2/CH3 and CD8α, and CD8α; a transmembrane domain derived from a polypeptide selected from the group consisting of: CD8α; CD4, CD45, PD1, and CD152; and one or more intracellular costimulatory signaling domains selected from the group consisting of: CD28, CD 134, and CD 137; and a CD3ζ primary signaling domain.

In yet another embodiment, CAR may contain an scFv, further including a linker, that binds an alpha folate receptor, 5T4, αvβ6 integrin, BCMA, B7-H3, B7-H6, CAIX, CD 19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, *Glypican-3 (GPC3), HLA-A1+ MAGE1, HLA-A2+ MAGE1, HLA-A3+ MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2 polypeptide; a hinge domain selected from the group consisting of: IgG1 hinge/CH2/CH3 and CD8α, and CD8α; a transmembrane domain comprising a TM domain derived from a polypeptide selected from the group consisting of: CD8α; CD4, CD45, PD1, and CD 152, and a short oligo- or polypeptide linker, preferably between 1, 2, 3, 4, 5, 6, 7, 8, 9, or 10 amino acids in length that links the TM domain to the intracellular signaling domain of the CAR; and one or more intracellular costimulatory signaling domains selected from the group consisting of: CD28, CD 134, and CD137; and a CD3ζ primary signaling domain.

In a particular embodiment, CAR may contain an scFv that binds an alpha folate receptor, 5T4, αvβ6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvIII, EGP2, EGP40, EPCAM, EphA2, EpCAM, FAP, fetal AchR, FRα, GD2, GD3, *Glypican-3 (GPC3), HLA-A1+ MAGE1, HLA-A2+M AGE1, HLA-A3+ MAGE1, HLA-A1+NY-ESO-1, HLA-A2+NY-ESO-1, HLA-A3+NY-ESO-1, IL-11Rα, IL-13Rα2, Lambda, Lewis-Y, Kappa, Mesothelin, Muc1, Muc16, NCAM, NKG2D Ligands, NY-ESO-1, PRAME, PSCA, PSMA, ROR1, SSX, Survivin, TAG72, TEMs, or VEGFR2 polypeptide; a hinge domain containing a CD8a polypeptide; a CD8a transmembrane domain containing a polypeptide linker of about 3 amino acids; one or more intracellular costimulatory signaling domains selected from the group consisting of: CD28, CD134, and CD137; and a CD3ζ primary signaling domain.

“Cytotoxic T lymphocyte” (CTL) as used herein refers broadly to a T lymphocyte that expresses CD8 on the surface thereof (e.g., a CD8+ T cell). Such cells may be preferably “memory” T cells (T_{M} cells) that are antigen-experienced.

“Donors” as used herein refers broadly human subjects that donated blood.

“Effective amount”, “therapeutically effective amount”, or “efficacious amount” as used herein refers broadly to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

“Genetically modified” as used herein refers broadly to methods to introduce exogenous nucleic acids into a cell, whether or not the exogenous nucleic acids are integrated into the genome of the cell.

“Genetically modified cell” as used herein refers broadly to cells that contain exogenous nucleic acids whether or not the exogenous nucleic acids are integrated into the genome of the cell.

“Immune cells” as used herein refers broadly to white blood cells (leukocytes) derived from hematopoietic stem cells (HSC) produced in the bone marrow “Immune cells” include, without limitation, lymphocytes (T cells, B cells, natural killer (NK) (CD3-CD56+) cells) and myeloid-derived cells (neutrophil, eosinophil, basophil, monocyte, macrophage, dendritic cells). “T cells” include all types of immune cells expressing CD3 including T-helper cells (CD4+ cells), cytotoxic T-cells (CD8+ cells), T-regulatory cells (Treg) and gamma-delta T cells, and NK T cells (CD3+ and CD56+). A skilled artisan will understand T cells and/or NK cells, as used throughout the disclosure, can include only T cells, only NK cells, or both T cells and NK cells. In certain illustrative embodiments and aspects provided herein, T cells are activated and transduced. Furthermore, T cells are provided in certain illustrative composition embodiments and aspects provided herein. A “cytotoxic cell” includes CD8+ T cells, natural-killer (NK) cells, NK-T cells, γδ T cells, and neutrophils, which are cells capable of mediating cytotoxicity responses.

“Individual,” “subject,” “host,” and “patient,” as used interchangeably herein, refer broadly to a mammal, including, but not limited to, humans, murines (e.g., rats, mice), lagomorphs (e.g., rabbits), non-human primates, canines, felines, and ungulates (e.g., equines, bovines, ovines, porcines, caprines).

“Peripheral blood mononuclear cells” or “PBMCs” as used herein refers broadly to any peripheral blood cell having a round nucleus. PBMCs include lymphocytes, such as T cells, B cells, and NK cells, and monocytes.

“Polynucleotide” and “nucleic acid”, as used interchangeably herein, refer broadly to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases.

“T cell” or “T lymphocyte” are art-recognized terms and include thymocytes, naïve T lymphocytes, immature T lymphocytes, mature T lymphocytes, resting T lymphocytes, or activated T lymphocytes. Illustrative populations of T cells suitable for use in particular embodiments include, but are not limited to, helper T cells (HTL; CD4+ T cell), a cytotoxic T cell (CTL; CD8+ T cell), CD4+CD8+ T cell, CD4-CD8− T cell, or any other subset of T cells. Other illustrative populations of T cells suitable for use in particular embodiments include, but are not limited to, T cells expressing one or more of the following markers: CD3, CD4, CD8, CD27, CD28, CD45RA, CD45RO, CD62L, CD127, CD197, and HLA-DR and if desired, can be further isolated by positive or negative selection techniques.

“T-cell receptor (TCR)” as used herein refers broadly to a protein receptor on T cells that is composed of a heterodimer of an alpha (α) and beta (β) chain, although in some cells the TCR consists of gamma and delta (γ/δ) chains. The TCR may be modified on any cell comprising a TCR, including a helper T cell, a cytotoxic T cell, a memory T cell, regulatory T cell, natural killer T cell, or a gamma delta T cell.

The TCR is generally found on the surface of T lymphocytes (or T cells) that is generally responsible for recognizing antigens bound to major histocompatibility complex (MHC) molecules. It is a heterodimer consisting of an alpha and beta chain in 95% of T cells, while 5% of T cells have TCRs consisting of gamma and delta chains. Engagement of the TCR with antigen and MHC results in activation of its T lymphocyte through a series of biochemical events mediated by associated enzymes, co-receptors, and specialized accessory molecules. In immunology, the CD3 antigen (CD stands for cluster of differentiation) is a protein complex composed of four distinct chains (CD3-γ, CD3δ, and two times CD3ε) in mammals, that associate with molecules known as the T-cell receptor (TCR) and the ζ-chain to generate an activation signal in T lymphocytes. The TCR, ζ-chain, and CD3 molecules together comprise the TCR complex. The CD3-γ, CD3δ, and CD3ε chains are highly related cell surface proteins of the immunoglobulin superfamily containing a single extracellular immunoglobulin domain. The transmembrane region of the CD3 chains is negatively charged, a characteristic that allows these chains to associate with the positively charged TCR chains (TCRα and TCRβ). The intracellular tails of the CD3 molecules contain a single conserved motif known as an immunoreceptor tyrosine-based activation motif or ITAM for short, which is essential for the signaling capacity of the TCR.

A T-cell may express TCRs and antigen binding proteins described in U.S. Patent Application Publication No. 2017/0267738; U.S. Patent Application Publication No. 2017/0312350; U.S. Patent Application Publication No. 2018/0051080; U.S. Patent Application Publication No. 2018/0164315; U.S. Patent Application Publication No. 2018/0161396; U.S. Patent Application Publication No. 2018/0162922; U.S. Patent Application Publication No. 2018/0273602; U.S. Patent Application Publication No. 2019/0016801; U.S. Patent Application Publication No. 2019/0002556; U.S. Patent Application Publication No. 2019/0135914; U.S. Pat. Nos. 10,538,573; 10,626,160; U.S. Patent Application Publication No. 2019/0321478; U.S. Patent Application Publication No. 2019/0256572; U.S. Pat. Nos. 10,550,182; 10,526,407; U.S. Patent Application Publication No. 2019/0284276; U.S. Patent Application Publication No. 2019/0016802; U.S. Patent Application Publication No. 2019/0016803; U.S. Patent Application Publication No. 2019/0016804; U.S. Pat. No. 10,583,573; U.S. Patent Application Publication No. 2020/0339652; U.S. Pat. Nos. 10,537,624; 10,596,242; U.S. Patent Application Publication No. 2020/0188497; U.S. Pat. No. 10,800,845; U.S. Patent Application Publication No. 2020/0385468; U.S. Pat. Nos. 10,527,623; 10,725,044; U.S. Patent Application Publication No. 2020/0249233; U.S. Pat. No. 10,702,609; U.S. Patent Application Publication No. 2020/0254106; U.S. Pat. No. 10,800,832; U.S. Patent Application Publication No. 2020/0123221; U.S. Pat. Nos. 10,590,194; 10,723,796; U.S. Patent Application Publication No. 2020/0140540; U.S. Pat. No. 10,618,956; U.S. Patent Application Publication No. 2020/0207849; U.S. Patent Application Publication No. 2020/0088726; and U.S. Patent Application Publication No. 2020/0384028; the contents of each of these publications and sequence listings described therein are herein incorporated by reference in their entireties. The T-cell may be a αβ T cell, γδ T cell, or a natural killer T cell.

Further, the TCRs may conatin an alpha chain (TCRα) and a beta chain (TCRβ). The TCRα chains and TCRβ chains that may be used in TCRs may be selected from R11KEA (SEQ ID NO: 162 and 163), R20P1H7 (SEQ ID NO: 164 and 165), R7P1D5 (SEQ ID NO: 166 and 167), R10P2G12 (SEQ ID NO: 168 and 169), R10P1A7 (SEQ ID NO: 170 and 171), R4P1D10 (SEQ ID NO: 172 and 173), R4P3F9 (SEQ ID NO: 174 and 175), R4P3H3 (SEQ ID NO: 176 and 177), R36P3F9 (SEQ ID NO: 178 and 179), R52P2G11 (SEQ ID NO: 180 and 181), R53P2A9 (SEQ ID NO: 182 and 183), R26P1A9 (SEQ ID NO: 184 and 185), R26P2A6 (SEQ ID NO: 186 and 187), R26P3H1 (SEQ ID NO: 188 and 189), R35P3A4 (SEQ ID NO: 190 and 191), R37P1C9 (SEQ ID NO: 192 and 193), R37P1H1 (SEQ ID NO: 194 and 195), R42P3A9 (SEQ ID NO: 196 and 197), R43P3F2 (SEQ ID NO: 198 and 199), R43P3G5 (SEQ ID NO: 200 and 201), R59P2E7 (SEQ ID NO: 202 and 203), R11P3D3 (SEQ ID NO: 204 and 205), R16P1C10 (SEQ ID NO: 206 and 207), R16P1E8 (SEQ ID NO: 208 and 209), R17P1A9 (SEQ ID NO: 210 and 211), R17P1D7 (SEQ ID NO: 212 and 213), R17P1G3 (SEQ ID NO: 214 and 215), R17P2B6 (SEQ ID NO: 216 and 217), R11P3D3KE (SEQ ID NO: 218 and 219), R39P1C12 (SEQ ID NO: 220 and 221), R39P1F5 (SEQ ID NO: 222 and 223), R40P1C2 (SEQ ID NO: 224 and 225), R41P3E6 (SEQ ID NO: 226 and 227), R43P3G4 (SEQ ID NO: 228 and 229), R44P3B3 (SEQ ID NO: 230 and 231), R44P3E7 (SEQ ID NO: 232 and 233), R49P2B7 (SEQ ID NO: 234 and 235), R55P1G7 (SEQ ID NO: 236 and 237), or R59P2A7 (SEQ ID NO: 238 and 239). The T-cell may be a αβ T cell, γδ T cell, or a natural killer T cell.

Table 1 shows examples of the peptides to which TCRs bind when the peptide is in a complex with an MHC molecule. (MHC molecules in humans may be referred to as HLA, human leukocyte-antigens).

TABLE 1
T-Cell Receptor and Peptides

| TCR name | Peptide (SEQ ID NO:) |
|---|---|
| R20P1H7, R7P1D5, R10P2G12 | KVLEHVVRV (SEQ ID NO: 118) |
| R10P1A7 | KIQEILTQV (SEQ ID NO: 240) |
| R4P1D10, R4P3F9, R4P3H3 | FLLDGSANV (SEQ ID NO: 141) |
| R36P3F9, R52P2G11, R53P2A9 | ILQDGQFLV (SEQ ID NO: 26) |
| R26P1A9, R26P2A6, R26P3H1, R35P3A4, | KVLEYVIKV (SEQ ID NO: 105) |
| R37P1C9, R37P1H1, R42P3A9, R43P3F2, | |
| R43P3G5, R59P2E7 | |
| R11KEA, R11P3D3, R16P1C10, R16P1E8, | SLLQHLIGL (SEQ ID NO: 50) |
| R17P1A9, R17P1D7, R17P1G3, R17P2B6, | |
| R11P3D3KE | |
| R39P1C12, R39P1F5, R40P1C2, R41P3E6, | ALSVLRLAL (SEQ ID NO: 151) |
| R43P3G4, R44P3B3, R44P3E7, R49P2B7, | |
| R55P1G7, R59P2A7 | |

TABLE 2
TCR sequences

| SEQ ID NO: | Description | Sequence |
|---|---|---|
| 162 | R11KEA alpha | MEKNPLAAPLLILWFHLDCVSSILNVEQSPQSLHVQEGD |
| | chain | STNFTCSFPSSNFYALHWYRKETAKSPEALFVMTLNGD |
| | | EKKKGRISATLNTKEGYSYLYIKGSQPEDSATYLCALYN |
| | | NNDMRFGAGTRLTVKPNIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 163 | R11KE beta | MDSWTFCCVSLCILVAKHTDAGVIQSPRHEVTEMGQEV |
| | chain | TLRCKPISGHNSLFWYRETMMRGLELLIYFNNNVPIDDS |
| | | GMPEDRFSAKMPNASFSTLKIQPSEPRDSAVYFCASSPG |
| | | STDTQYFGPGTRLTVLEDLKNVFPPEVAVFEPSEAEISHT |
| | | QKATLVCLATGFYPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSES |
| | | YQQGVLSATILYEILLGKATLYAVLVSALVLMAMVKR |
| | | KDSRG |
| 164 | R20P1H7 alpha | MEKMLECAFIVLWLQLGWLSGEDQVTQSPEALRLQEG |
| | chain | ESSSLNCSYTVSGLRGLFWYRQDPGKGPEFLFTLYSAGE |
| | | EKEKERLKATLTKKESFLHITAPKPEDSATYLCAVQGEN |
| | | SGYSTLTFGKGTMLLVSPDIQNPDPAVYQLRDSKSSDKS |
| | | VCLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 165 | R20P1H7 beta | MGPQLLGYVVLCLLGAGPLEAQVTQNPRYLITVTGKKL |
| | chain | TVTCSQNMNHEYMSWYRQDPGLGLRQIYYSMNVEVT |
| | | DKGDVPEGYKVSRKEKRNFPLILESPSPNQTSLYFCASS |
| | | LGPGLAAYNEQFFGPGTRLTVLEDLKNVFPPEVAVFEPS |
| | | EAEISHTQKATLVCLATGFYPDHVELSWWVNGKEVHS |
| | | GVSTDPQPLKEQPALNDSRYCLSSRLRVSATFWQNPRN |
| | | HFRCQVQFYGLSENDEWTQDRAKPVTQIVSAEAWGRA |
| | | DCGFTSESYQQGVLSATILYEILLGKATLYAVLVSALVL |
| | | MAMVKRKDSRG |
| 166 | R7P1D5 alpha | MKTFAGFSFLFLWLQLDCMSRGEDVEQSLFLSVREGDS |
| | chain | SVINICTYTDSSSTYLYWYKQEPGAGLQLLTYIFSNMDM |
| | | KQDQRLTVLLNKKDKHLSLRIADTQTGDSAIYFCAEYS |
| | | SASKIIFGSGTRLSRPNIQNPDPAVYQLRDSKSSDKSVC |
| | | LFTDFDSQTNVSQSKDSDVYITDKTVLDMRSMDFKSNS |
| | | AVAWSNKSDFACANAFNNSIIPEDTFFPSPESSCDVKLV |
| | | EKSFETDTNLNFQNLSVIGFRILLLKVAGFNLLMTLRLW |
| | | SS |
| 167 | R7P1D5 beta | MGSWTLCCVSLCILVAKHTDAGVIQSPRHEVTEMGQEV |
| | chain | TLRCKPISGHDYLFWYRQTMMRGLELLIYFNNNVPIDD |
| | | SGMPEDRFSAKMPNASFSTLKIQPSEPRDSAVYFCASRA |
| | | NTGELFFGEGSRLTVLEDLKNVFPPEVAVFEPSEAEISHT |
| | | QKATLVCLATGFYPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSES |
| | | YQQGVLSATILYEILLGKATLYAVLVSALVLMAMVKR |
| | | KDSRG |
| 168 | R10P2G12 alpha | MLTASLLRAVIASICVVSSMAQKVTQAQTEISVVEKED |
| | chain | VTLDCVYETRDTTYYLFWYKQPPSGELVFLIRRNSFDE |
| | | QNEISGRYSWNFQKSTSSFNFTITASQVVDSAVYFCALS |
| | | EGNSGNTPLVFGKGTRLSVIANIQNPDPAVYQLRDSKSS |
| | | DKSVCLFTDFDSQTNVSQSKDSDVYITDKTVLDMRSMD |
| | | FKSNSAVAWSNKSDFACANAFNNSIIPEDTFFPSPESSCD |
| | | VKLVEKSFETDTNLNFQNLSVIGFRILLLKVAGFNLLMT |
| | | LRLWSS |
| 169 | R10P2G12 beta | MGIRLLCRVAFCFLAVGLVDVKVTQSSRYLVKRTGEKV |
| | chain | FLECVQDMDHENMFWYRQDPGLGLRLIYFSYDVKMKE |
| | | KGDIPEGYSVSREKKERFSLILESASTNQTSMYLCASSLS |
| | | SGSHQETQYFGPGTRLLVLEDLKNVFPPEVAVFEPSEAE |
| | | ISHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADCG |
| | | FTSESYQQGVLSATILYEILLGKATLYAVLVSALVLMA |
| | | MVKRKDSRG |
| 170 | R10P1A7 alpha | MKTFAGFSFLFLWLQLDCMSRGEDVEQSLFLSVREGDS |
| | chain | SVINCTYTDSSSTYLYWYKQEPGAGLQLLTYIFSNMDM |
| | | KQDQRLTVLLNKKDKHLSLRIADTQTGDSAIYFCAESK |
| | | ETRLMFGDGTQLVVKPNIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 171 | R10P1A7 beta | MLLLLLLLGPGISLLLPGSLAGSGLGAWSQHPSVWICKS |
| | chain | GTSVKIECRSLDFQATTMFWYRQFPKQSLMLMATSNEG |
| | | SKATYEQGVEKDKFLINHASLTLSTLTVTSAHPEDSSFYI |
| | | CSARAGGHEQFFGPGTRLTVLEDLKNVFPPEVAVFEPSE |
| | | AEISHTQKATLVCLATGFYPDHVELSWVWNGKEVHSG |
| | | VSTDPQPLKEQPALNDSRYCLSSRLRVSATFWQNPRNH |
| | | FRCQVQFYGLSENDEWTQDRAKPVTQIVSAEAWGRAD |
| | | CGFTSESYQQGVLSATILYEILLGKATLYAVLVSALVLM |
| | | AMVKRKDSRG |
| 172 | R4P1D10 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVNF |
| | | HDKIIFGKGTRLHILPNIQNPDPAVYQLRDSKSSDKSVCL |
| | | FTDFDSQTNVSQSKDSDVYITDKTVLDMRSMDFKSNSA |
| | | VAWSNKSDFACANAFNNSIIPEDTFFPSPESSCDVKLVE |
| | | KSFETDTNLNFQNLSVIGFRILLLKVAGFNLLMTLRLWS |
| | | S |
| 173 | R4P1D10 beta | MGFRLLCCVAFCLLGAGPVDSGVTQTPKHLITATGQRV |
| | chain | TLRCSPRSGDLSVYWYQQSLDQGLQFLIHYYNGEERAK |
| | | GNILERFSAQQFPDLHSELNLSSLELGDSALYFCASSVAS |
| | | AYGYTFGSGTRLTVVEDLNKVFPPEVAVFEPSEAEISHT |
| | | QKATLVCLATGFFPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSV |
| | | SYQQGVLSATILYEILLGKATLYAVLVSALVLMAMVKR |
| | | KDF |
| 174 | R4P3F9 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAAYS |
| | | GAGSYQLTFGKGTKLSVIPNIQNPDPAVYQLRDSKSSDK |
| | | SVCLFTDFDSQTNVSQSKDSDVYITDKTVLDMRSMDFK |
| | | SNSAVAWSNKSDFACANAFNNSIIPEDTFFPSPESSCDV |
| | | KLVEKSFETDTNLNFQNLSVIGFRILLLKVAGFNLLMTL |
| | | RLWSS |
| 175 | R4P3F9 beta | MGFRLLCCVAFCLLGAGPVDSGVTQTPKHLITATGQRV |
| | chain | TLRCSPRSGDLSVYWYQQSLDQGLQFLIQYYNGEERAK |
| | | GNILERFSAQQFPDLHSELNLSSLELGDSALYFCASSVES |
| | | SYGYTFGSGTRLTVVEDLNKVFPPEVAVFEPSEAEISHT |
| | | QKATLVCLATGFFPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSV |
| | | SYQQGVLSATILYEILLGKATLYAVLVSALVLMAMVKR |
| | | KDF |
| 176 | R4P3H3 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVKA |
| | | GNQFYFGTGTSLTVIPNIQNPDPAVYQLRDSKSSDKSVC |
| | | LFTDFDSQTNVSQSKDSDVYITDKTVLDMRSMDFKSNS |
| | | AVAWSNKSDFACANAFNNSIIPEDTFFPSPESSCDVKLV |
| | | EKSFETDTNLNFQNLSVIGFRILLLKVAGFNLLMTLRLW |
| | | SS |
| 177 | R4P3H3 beta | MGTRLLCWVVLGFLGTDHTGAGVSQSPRYKVAKRGQ |
| | chain | DVALRCDPISGHVSLFWYQQALGQGPEFLTYFQNEAQL |
| | | DKSGLPSDRFFAERPEGSVSTLKIQRTQQEDSAVYLCAS |
| | | SLLTSGGDNEQFFGPGTRLTVLEDLKNVFPPEVAVFEPS |
| | | EAEISHTQKATLVCLATGFYPDHVELSWWVNGKEVHS |
| | | GVSTDPQPLKEQPALNDSRYCLSSRLRVSATFWQNPRN |
| | | HFRCQVQFYGLSENDEWTQDRAKPVTQIVSAEAWGRA |
| | | DCGFTSESYQQGVLSATILYEILLGKATLYAVLVSALVL |
| | | MAMVKRKDSRG |
| 178 | R36P3F9 alpha | METLLGVSLVILWLQLARVNSQQGEEDPQALSIQEGEN |
| | chain | ATMNCSYKTSINNLQWYRQNSGRGLVHLILIRSNEREK |
| | | HSGRLRVTLDTSKKSSSLLITASRAADTASYFCATVSNY |
| | | QLIWGAGTKLIIKPDIQNPDPAVYQLRDSKSSDKSVCLF |
| | | TDFDSQTNVSQSKDSDVYITDKTVLDMRSMDFKSNSAV |
| | | AWSNKSDFACANAFNNSIIPEDTFFPSPESSCDVKLVEKS |
| | | FETDTNLNFQNLSVIGFRILLLKVAGFNLLMTLRLWSS |
| 179 | R36P3F9 beta | MGPQLLGYVVLCLLGAGPLEAQVTQNPRYLITVTGKKL |
| | chain | TVTCSQNMNHEYMSWYRQDPGLGLRQIYYSMNVEVT |
| | | DKGDVPEGYKVSRKEKRNFPLILESPSPNQTSLYFCASS |
| | | STSGGLSGETQYFGPGTRLLVLEDLKNVFPPEVAVFEPS |
| | | EAEISHTQKATLVCLATGFYPDHVELSWWVNGKEVHS |
| | | GVSTDPQPLKEQPALNDSRYCLSSRLRVSATFWQNPRN |
| | | HFRCQVQFYGLSENDEWTQDRAKPVTQIVSAEAWGRA |
| | | DCGFTSESYQQGVLSATILYEILLGKATLYAVLVSALVL |
| | | MAMVKRKDSRG |
| 180 | R52P2G11 alpha | MKKHLTTFLVILWLYFYRGNGKNQVEQSPQSLIILEGK |
| | chain | NCTLQCNYTVSPFSNLRWYKQDTGRGPVSLTIMTFSEN |
| | | TKSNGRYTATLDADTKQSSLHITASQLSDSASYICVVSA |
| | | YGKLQFGAGTQVVVTPDIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 181 | R52P2G11 beta | MDSWTFCCVSLCILVAKHTDAGVIQSPRHEVTEMGQEV |
| | chain | TLRCKPISGHNSLFWYRQTMMRGLELLIYFNNNVPIDDS |
| | | GMPEDRFSAKMPNASFSTLKIQPSEPRDSAVYFCASSLG |
| | | SPDGNQPQHFGDGTRLSILEDLNKVFPPEVAVFEPSEAEI |
| | | SHTQKATLVCLATGFFPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSVSYQQGVLSATILYEILLGKATLYAVLVSALVLMAM |
| | | VKRKDF |
| 182 | R53P2A9 alpha | MACPGFLWALVISTCLEFSMAQTVTQSQPEMSVQEAET |
| | chain | VTLSCTYDTSESDYYLFWYKQPPSRQMILVIRQEAYKQ |
| | | QNATENRFSVNFQKAAKSFSLKISDSQLGDAAMYFCAY |
| | | NSYAGGTSYGKLTFGQGTILTVHPNIQNPDPAVYQLRD |
| | | SKSSDKSVCLFTDFDSQTNVSQSKDSDVYITDKTVLDM |
| | | RSMDFKSNSAVAWSNKSDFACANAFNNSIIPEDTFFPSP |
| | | ESSCDVKLVEKSFETDTNLNFQNLSVIGFRILLLKVAGF |
| | | NLLMTLRLWSS |
| 183 | R53P2A9 beta | MGPGLLCWVLLCLLGAGPVDAGVTQSPTHLIKTRGQQ |
| | chain | VTLRCSPISGHKSVSWYQQVLGQGPQFIFQYYEKEERG |
| | | RGNFPDRFSARQFPNYSSELNVNALLLGDSALYLCASSL |
| | | DGTSEQYFGPGTRLTVTEDLKNVFPPEVAVFEPSEAEIS |
| | | HTQKATLVCLATGFYPDHVELSWWVNGKEVHSGVSTD |
| | | PQPLKEQPALNDSRYCLSSRLRVSATFWQNPRNHFRCQ |
| | | VQFYGLSENDEWTQDRAKPVTQIVSAEAWGRADCGFT |
| | | SESYQQGVLSATILYEILLGKATLYAVLVSALVLMAMV |
| | | KRKDSRG |
| 184 | R26P1A9 alpha | METLLGVSLVILWLQLARVNSQQGEEDPQALSIQEGEN |
| | chain | ATMNCSYKTSINNLQWYRQNSGRGLVHLILIRSNEREK |
| | | HSGRLRVTLDTSKKSSSLLITASRAADTASYFCLIGASGS |
| | | RLTFGEGTQLTVNPDIQNPDPAVYQLRDSKSSDKSVCLF |
| | | TDFDSQTNVSQSKDSDVYITDKTVLDMRSMDFKSNSAV |
| | | AWSNKSDFACANAFNNSIIPEDTFFPSPESSCDVKLVEKS |
| | | FETDTNLNFQNLSVIGFRILLLKVAGFNLLMTLRLWSS |
| 185 | R26P1A9 beta | MGSWTLCCVSLCILVAKHTDAGVIQSPRHEVTEMGQEV |
| | chain | TLRCKPISGHDYLFWYRQTMMRGLELLIYFNNNVPIDD |
| | | SGMPEDRFSAKMPNASFSTLKIQPSEPRDSAVYFCASSY |
| | | FGWNEKLFFGSGTQLSVLEDLNKVFPPEVAVFEPSEAEI |
| | | SHTQKATLVCLATGFFPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSVSYQQGVLSATILYEILLGKATLYAVLVSALVLMAM |
| | | VKRKDF |
| 186 | R26P2A6 alpha | MMKSLRVLLVILWLQLSWVWSQQKEVEQDPGPLSVPE |
| | chain | GAIVSLNCTYSNSAFQYFMWYRQYSRKGPELLMYTYSS |
| | | GNKEDGRFTAQVDKSSKYISLFIRDSQPSDSATYLCAMS |
| | | DVSGGYNKLIFGAGTRLAVHPYIQNPDPAVYQLRDSKS |
| | | SDKSVCLFTDFDSQTNVSQSKDSDVYITDKTVLDMRSM |
| | | DFKSNSAVAWSNKSDFACANAFNNSIIPEDTFFPSPESSC |
| | | DVKLVEKSFETDTNLNFQNLSVIGFRILLLKVAGFNLLM |
| | | TLRLWSS |
| 187 | R26P2A6 beta | MGPQLLGYVVLCLLGAGPLEAQVTQNPRYLITVTGKKL |
| | chain | TVTCSQNMNHEYMSWYRQDPGLGLRQIYYSMNVEVT |
| | | DKGDVPEGYKVSRKEKRNFPLILESPSPNQTSLYFCAST |
| | | TPDGTDEQFFGPGTRLTVLEDLKNVFPPEVAVFEPSEAEI |
| | | SHTQKATLVCLATGFYPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSESYQQGVLSATILYEILLGKATLYAVLVSALVLMAM |
| | | VKRKDSRG |
| 188 | R26P3H1 alpha | MASAPISMLAMLFTLSGLRAQSVAQPEDQVNVAEGNPL |
| | chain | TVKCTYSVSGNPYLFWYVQYPNRGLQFLLKYITGDNLV |
| | | KGSYGFEAEFNKSQTSFHLKKPSALVSDSALYFCAVRD |
| | | MNRDDKIIFGKGTRLHILPNIQNPDPAVYQLRDSKSSDK |
| | | SVCLFTDFDSQTNVSQSKDSDVYITDKTVLDMRSMDFK |
| | | SNSAVAWSNKSDFACANAFNNSIIPEDTFFPSPESSCDV |
| | | KLVEKSFETDTNLNFQNLSVIGFRILLLKVAGFNLLMTL |
| | | RLWSS |
| 189 | R26P3H1 beta | MSNQVLCCVVLCFLGANTVDGGITQSPKYLFRKEGQN |
| | chain | VTLSCEQNLNHDAMYWYRQDPGQGLRLIYYSQIVNDF |
| | | QKGDIAEGYSVSREKKESFPLTVTSAQKNPTAFYLCASS |
| | | RAEGGEQYFGPGTRLTVTEDLKNVFPPEVAVFEPSEAEI |
| | | SHTQKATLVCLATGFYPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSESYQQGVLSATILYEILLGKATLYAVLVSALVLMAM |
| | | VKRKDSRG |
| 190 | R35P3A4 alpha | MTSIRAVFIFLWLQLDLVNGENVEQHPSTLSVQEGDSA |
| | chain | VIKCTYSDSASNYFPWYKQELGKRPQLIIDIRSNVGEKK |
| | | DQRIAVTLNKTAKHFSLHITETQPEDSAVYFCAASPTGG |
| | | YNKLIFGAGTRLAVHPYIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 191 | R35P3A4 beta | MSIGLLCCAALSLLWAGPVNAGVTQTPKFQVLKTGQS |
| | chain | MTLQCAQDMNHEYMSWYRQDPGMGLRLIHYSVGAGI |
| | | TDQGEVPNGYNVSRSTTEDFPLRLLSAAPSQTSVYFCAS |
| | | SLGGASQEQYFGPGTRLTVTEDLKNVFPPEVAVFEPSEA |
| | | EISHTQKATLVCLATGFYPDHVELSWWVNGKEVHSGV |
| | | STDPQPLKEQPALNDSRYCLSSRLRVSATFWQNPRNHF |
| | | RCQVQFYGLSENDEWTQDRAKPVTQIVSAEAWGRADC |
| | | GFTSESYQQGVLSATILYEILLGKATLYAVLVSALVLMA |
| | | MVKRKDSRG |
| 192 | R37P1C9 alpha | MKLVTSITVLLSLGIMGDAKTTQPNSMESNEEEPVHLPC |
| | chain | NHSTISGTDYIHWYRQLPSQGPEYVIHGLTSNVNNRMA |
| | | SLAIAEDRKSSTLILHRATLRDAAVYYCILFNFNKFYFGS |
| | | GTKLNVKPNIQNPDPAVYQLRDSKSSDKSVCLFTDFDS |
| | | QTNVSQSKDSDVYITDKTVLDMRSMDFKSNSAVAWSN |
| | | KSDFACANAFNNSIIPEDTFFPSPESSCDVKLVEKSFETD |
| | | TNLNFQNLSVIGFRILLLKVAGFNLLMTLRLWSS |
| 193 | R37P1C9 beta | MGPGLLHWMALCLLGTGHGDAMVIQNPRYQVTQFGK |
| | chain | PVTLSCSQTLNHNVMYWYQQKSSQAPKLLFHYYDKDF |
| | | NNEADTPDNFQSRRPNTSFCFLDIRSPGLGDAAMYLCA |
| | | TSSGETNEKLFFGSGTQLSVLEDLNKVFPPEVAVFEPSE |
| | | AEISHTQKATLVCLATGFFPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRAD |
| | | CGFTSVSYQQGVLSATILYEILLGKATLYAVLVSALVLM |
| | | AMVKRKDF |
| 194 | R37P1H1 alpha | MTRVSLLWAVVVSTCLESGMAQTVTQSQPEMSVQEAE |
| | chain | TVTLSCTYDTSESNYYLFWYKQPPSRQMILVIRQEAYK |
| | | QQNATENRFSVNFQKAAKSFSLKISDSQLGDTAMYFCA |
| | | FGYSGGGADGLTFGKGTHLIIQPYIQNPDPAVYQLRDSK |
| | | SSDKSVCLFTDFDSQTNVSQSKDSDVYITDKTVLDMRS |
| | | MDFKSNSAVAWSNKSDFACANAFNNSIIPEDTFFPSPES |
| | | SCDVKLVEKSFETDTNLNFQNLSVIGFRILLLKVAGFNL |
| | | LMTLRLWSS |
| 195 | R37P1H1 beta | MGPGLLCWALLCLLGAGLVDAGVTQSPTHLIKTRGQQ |
| | chain | VTLRCSPKSGHDTVSWYQQALGQGPQFIFQYYEEEERQ |
| | | RGNFPDRFSGHQFPNYSSELNVNALLLGDSALYLCASS |
| | | NEGQGWEAEAFFGQGTRLTVVEDLNKVFPPEVAVFEPS |
| | | EAEISHTQKATLVCLATGFFPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRAD |
| | | CGFTSVSYQQGVLSATILYEILLGKATLYAVLVSALVLM |
| | | AMVKRKDF |
| 196 | R42P3A9 alpha | MKRILGALLGLLSAQVCCVRGIQVEQSPPDLILQEGANS |
| | chain | TLRCNFSDSVNNLQWFHQNPWGQLINLFYIPSGTKQNG |
| | | RLSATTVATERYSLLYISSSQTTDSGVYFCAVHNFNKFY |
| | | FGSGTKLNVKPNIQNPDPAVYQLRDSKSSDKSVCLFTDF |
| | | DSQTNVSQSKDSDVYITDKTVLDMRSMDFKSNSAVAW |
| | | SNKSDFACANAFNNSIIPEDTFFPSPESSCDVKLVEKSFE |
| | | TDTNLNFQNLSVIGFRILLLKVAGFNLLMTLRLWSS |
| 197 | R42P3A9 beta | MLSPDLPDSAWNTRLLCHVMLCLLGAVSVAAGVIQSPR |
| | chain | HLIKEKRETATLKCYPIPRHDTVYWYQQGPGQDPQFLIS |
| | | FYEKMQSDKGSIPDRFSAQQFSDYHSELNMSSLELGDS |
| | | ALYFCASSLLGQGYNEQFFGPGTRLTVLEDLKNVFPPEV |
| | | AVFEPSEAEISHTQKATLVCLATGFYPDHVELSWWVNG |
| | | KEVHSGVSTDPQPLKEQPALNDSRYCLSSRLRVSATFW |
| | | QNPRNHFRCQVQFYGLSENDEWTQDRAKPVTQIVSAE |
| | | AWGRADCGFTSESYQQGVLSATILYEILLGKATLYAVL |
| | | VSALVLMAMVKRKDSRG |
| 198 | R43P3F2 alpha | MLTASLLRAVIASICVVSSMAQKVTQAQTEISVVEKED |
| | chain | VTLDCVYETRDTTYYLFWYKQPPSGELVFLIRRNSFDE |
| | | QNEISGRYSWNFQKSTSSFNFTITASQVVDSAVYFCALS |
| | | NNNAGNMLTFGGGTRLMVKPHIQNPDPAVYQLRDSKS |
| | | SDKSVCLFTDFDSQTNVSQSKDSDVYITDKTVLDMRSM |
| | | DFKSNSAVAWSNKSDFACANAFNNSIIPEDTFFPSPESSC |
| | | DVKLVEKSFETDTNLNFQNLSVIGFRILLLKVAGFNLLM |
| | | TLRLWSS |
| 199 | R43P3F2 beta | MLSPDLPDSAWNTRLLCHVMLCLLGAVSVAAGVIQSPR |
| | chain | HLIKEKRETATLKCYPIPRHDTVYWYQQGPGQDPQFLIS |
| | | FYEKMQSDKGSIPDRFSAQQFSDYHSELNMSSLELGDS |
| | | ALYFCASSPTGTSGYNEQFFGPGTRLTVLEDLKNVFPPE |
| | | VAVFEPSEAEISHTQKATLVCLATGFYPDHVELSWWVN |
| | | GKEVHSGVSTDPQPLKEQPALNDSRYCLSSRLRVSATF |
| | | WQNPRNHFRCQVQFYGLSENDEWTQDRAKPVTQIVSA |
| | | EAWGRADCGFTSESYQQGVLSATILYEILLGKATLYAV |
| | | LVSALVLMAMVKRKDSRG |
| 200 | R43P3G5 alpha | MEKNPLAAPLLILWFHLDCVSSILNVEQSPQSLHVQEGD |
| | chain | STNFTCSFPSSNFYALHWYRWETAKSPEALFVMTLNGD |
| | | EKKKGRISATLNTKEGYSYLYIKGSQPEDSATYLCALNR |
| | | DDKIIFGKGTRLHILPNIQNPDPAVYQLRDSKSSDKSVCL |
| | | FTDFDSQTNVSQSKDSDVYITDKTVLDMRSMDFKSNSA |
| | | VAWSNKSDFACANAFNNSIIPEDTFFPSPESSCDVKLVE |
| | | KSFETDTNLNFQNLSVIGFRILLLKVAGFNLLMTLRLWS |
| | | S |
| 201 | R43P3G5 beta | MGIRLLCRVAFCFLAVGLVDVKVTQSSRYLVKRTGEKV |
| | chain | FLECVQDMDHENMFWYRQDPGLGLRLIYFSYDVKMKE |
| | | KGDIPEGYSVSREKKERFSLILESASTNQTSMYLCASRLP |
| | | SRTYEQYFGPGTRLTVTEDLKNVFPPEVAVFEPSEAEIS |
| | | HTQKATLVCLATGFYPDHVELSWWVNGKEVHSGVSTD |
| | | PQPLKEQPALNDSRYCLSSRLRVSATFWQNPRNHFRCQ |
| | | VQFYGLSENDEWTQDRAKPVTQIVSAEAWGRADCGFT |
| | | SESYQQGVLSATILYEILLGKATLYAVLVSALVLMAMV |
| | | KRKDSRG |
| 202 | R59P2E7 alpha | METLLGLLILWLQLQWVSSKQEVTQIPAALSVPEGENL |
| | chain | VLNCSFTDSAIYNLQWFRQDPGKGLTSLLLIQSSQREQT |
| | | SGRLNASLDKSSGRSTLYIAASQPGDSATYLCAVNSDY |
| | | KLSFGAGTTVTVRANIQNPDPAVYQLRDSKSSDKSVCL |
| | | FTDFDSQTNVSQSKDSDVYITDKTVLDMRSMDFKSNSA |
| | | VAWSNKSDFACANAFNNSIIPEDTFFPSPESSCDVKLVE |
| | | KSFETDTNLNFQNLSVIGFRILLLKVAGFNLLMTLRLWS |
| | | S |
| 203 | R59P2E7 beta | MLSPDLPDSAWNTRLLCHVMLCLLGAVSVAAGVIQSPR |
| | chain | HLIKEKRETATLKCYPIPRHDTVYWYQQGPGQDPQFLIS |
| | | FYEKMQSDKGSIPDRFSAQQFSDYHSELNMSSLELGDS |
| | | ALYFCASSLGLGTGDYGYTFGSGTRLTVVEDLNKVFPP |
| | | EVAVFEPSEAEISHTQKATLVCLATGFFPDHVELSWWV |
| | | NGKEVHSGVSTDPQPLKEQPALNDSRYCLSSRLRVSAT |
| | | FWQNPRNHFRCQVQFYGLSENDEWTQDRAKPVTQIVS |
| | | AEAWGRADCGFTSVSYQQGVLSATILYEILLGKATLYA |
| | | VLVSALVLMAMVKRKDF |
| 204 | R11P3D3 alpha | MEKNPLAAPLLILWFHLDCVSSILNVEQSPQSLHVQEGD |
| | chain | STNFTCSFPSSNFYALHWYRWETAKSPEALFVMTLNGD |
| | | EKKKGRISATLNTKEGYSYLYIKGSQPEDSATYLCALYN |
| | | NNDMRFGAGTRLTVKPNIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 205 | R11P3D3 beta | MDSWTFCCVSLCILVAKHTDAGVIQSPRHEVTEMGQEV |
| | chain | TLRCKPISGHNSLFWYRQTMMRGLELLIYFNNNVPIDDS |
| | | GMPEDRFSAKMPNASFSTLKIQPSEPRDSAVYFCASSPG |
| | | STDTQYFGPGTRLTVLEDLKNVFPPEVAVFEPSEAEISHT |
| | | QKATLVCLATGFYPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSES |
| | | YQQGVLSAT1LYEILLGKATLYAVLVSALVLMAMVKRK |
| | | DSRG |
| 206 | R16P1C10 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAAVIS |
| | | NFGNEKLTFGTGTRLTIIPNIQNPDPAVYQLRDSKSSDKS |
| | | VCLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 207 | R16P1C10 beta | MGSRLLCWVLLCLLGAGPVKAGVTQTPRYLIKTRGQQ |
| | chain | VTLSCSPISGHRSVSWYQQTPGQGLQFLFEYFSETQRNK |
| | | GNFPGRFSGRQFSNSRSEMNVSTLELGDSALYLCASSP |
| | | WDSPNEQYFGPGTRLTVTEDLKNVFPPEVAVFEPSEAEI |
| | | SHTQKATLVCLATGFYPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSESYQQGVLSATILYEILLGKATLYAVLVSALVLMAM |
| | | VKRKDSRG |
| 208 | R16P1E8 alpha | MMKSLRVLLVILWLQLSWVWSQQKEVEQDPGPLSVPE |
| | chain | GAIVSLNCTYSNSAFQYFMWYRQYSRKGPELLMYTYSS |
| | | GNKEDGRFTAQVDKSSKYISLFIRDSQPSDSATYLCAMS |
| | | EAAGNKLTFGGGTRVLVKPNIQNPDPAVYQLRDSKSSD |
| | | KSVCLFTDFDSQTNVSQSKDSDVYITDKTVLDMRSMDF |
| | | KSNSAVAWSNKSDFACANAFNNSIIPEDTFFPSPESSCD |
| | | VKLVEKSFETDTNLNFQNLSVIGFRILLLKVAGFNLLMT |
| | | LRLWSS |
| 209 | R16P1E8 beta | MGTRLLCWAALCLLGAELTEAGVAQSPRYKIIEKRQSV |
| | chain | AFWCNPISGHATLYWYQQILGQGPKLLIQFQNNGVVDD |
| | | SQLPKDRFSAERLKGVDSTLKIQPAKLEDSAVYLCASSY |
| | | TNQGEAFFGQGTRLTVVEDLNKVFPPEVAVFEPSEAEIS |
| | | HTQKATLVCLATGFFPDHVELSWWVNGKEVHSGVSTD |
| | | PQPLKEQPALNDSRYCLSSRLRVSATFWQNPRNHFRCQ |
| | | VQFYGLSENDEWTQDRAKPVTQIVSAEAWGRADCGFT |
| | | SVSYQQGVLSATILYEILLGKATLYAVLVSALVLMAMV |
| | | KRKDF |
| 210 | R17P1A9 alpha | MKSLRVLLVILWLQLSWVWSQQKEVEQNSGPLSVPEG |
| | chain | AIASLNCTYSDRGSQSFFWYRQYSGKSPELIMSIYSNGD |
| | | KEDGRFTAQLNKASQYVSLLIRDSQPSDSATYLCAVLN |
| | | QAGTALIFGKGTTLSVSSNIQNPDPAVYQLRDSKSSDKS |
| | | VCLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 211 | R17P1A9 beta | MGFRLLCCVAFCLLGAGPVDSGVTQTPKHLITATGQRV |
| | chain | TLRCSPRSGDLSVYWYQQSLDQGLQFLIQYYNGEERAK |
| | | GNILERFSAQQFPDLHSELNLSSLELGDSALYFCASSAET |
| | | GPWLGNEQFFGPGTRLTVLEDLKNVFPPEVAVFEPSEAE |
| | | ISHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADCG |
| | | FTSESYQQGVLSATILYEILLGKATLYAVLVSALVLMA |
| | | MVKRKDSRG |
| 212 | R17P1D7 alpha | MACPGFLWALVISTCLEFSMAQTVTQSQPEMSVQEAET |
| | chain | VTLSCTYDTSESDYYLFWYKQPPSRQMILVIRQEAYKQ |
| | | QNATENRFSVNFQKAAKSFSLKISDSQLGDAAMYFCAY |
| | | RWAQGGSEKLVFGKGTKLTVNPYIQKPDPAVYQLRDS |
| | | KSSDKSVCLFTDFDSQTNVSQSKDSDVYITDKTVLDMR |
| | | SMDFKSNSAVAWSNKSDFACANAFNNSIIPEDTFFPSPE |
| | | SSCDVKLVEKSFETDTNLNFQNLSVIGFRILLLKVAGFN |
| | | LLMTLRLWSS |
| 213 | R17P1D7 beta | MTIRLLCYMGFYFLGAGLMEADIYQTPRYLVIGTGKKIT |
| | chain | LECSQTMGHDKMYWYQQDPGMELHLIHYSYGVNSTE |
| | | KGDLSSESTVSRIRTEHFPLTLESARPSHTSQYLCATELW |
| | | SSGGTGELFFGEGSRLTVLEDLKNVFPPEVAVFEPSEAEI |
| | | SHTQKATLVCLATGFYPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSESYQQGVLSATILYEILLGKATLYAVLVSALVLMAM |
| | | VKRKDSRG |
| 214 | R17P1G3 alpha | MKSLRVLLVILWLQLSWVWSQQKEVEQNSGPLSVPEG |
| | chain | AIASLNCTYSDRGSQSFFWYRQYSGKSPELIMSIYSNGD |
| | | KEDGRFTAQLNKASQYVSLLIRDSQPSDSATYLCAVGPS |
| | | GTYKYIFGTGTRLKVLANIQNPDPAVYQLRDSKSSDKS |
| | | VCLFTDFDSQTNVSQSKDSDVYITDKTVLDMRSMDFKS |
| | | NSAVAWSNKSDFACANAFNNSIIPEDTFFPSPESSCDVK |
| | | LVEKSFETDTNLNFQNLSVIGFRILLLKVAGFNLLMTLR |
| | | LWSS |
| 215 | R17P1G3 beta | MGPQLLGYVVLCLLGAGPLEAQVTQNPRYLITVTGKKL |
| | chain | TVTCSQNMNHEYMSWYRQDPGLGLRQIYYSMNVEVT |
| | | DKGDVPEGYKVSRKEKRNFPLILESPSPNQTSLYFCASS |
| | | PGGSGNEQFFGPGTRLTVLEDLKNVFPPEVAVFEPSEAE |
| | | ISHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADCG |
| | | FTSESYQQGVLSATILYEILLGKATLYAVLVSALVLMA |
| | | MVKRKDSRG |
| 216 | R17P2B6 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVVS |
| | | GGGADGLTFGKGTHLIIQPYIQKPDPAVYQLRDSKSSDK |
| | | SVCLFTDFDSQTNVSQSKDSDVYITDKTVLDMRSMDFK |
| | | SNSAVAWSNKSDFACANAFNNSIIPEDTFFPSPESSCDV |
| | | KLVEKSFETDTNLNFQNLSVIGFRILLLKVAGFNLLMTL |
| | | RLWSS |
| 217 | R17P2B6 beta | MLSPDLPDSAWNTRLLCHVMLCLLGAVSVAAGVIQSPR |
| | chain | HLIKEKRETATLKCYPIPRHDTVYWYQQGPGQDPQFLIS |
| | | FYEKMQSDKGSIPDRFSAQQFSDYHSELNMSSLELGDS |
| | | ALYFCASSLGRGGQPQHFGDGTRLSILEDLNKVFPPEVA |
| | | VFEPSEAEISHTQKATLVCLATGFFPDHVELSWWVNGK |
| | | EVHSGVSTDPQPLKEQPALNDSRYCLSSRLRVSATFWQ |
| | | NPRNHFRCQVQFYGLSENDEWTQDRAKPVTQIVSAEA |
| | | WGRADCGFTSVSYQQGVLSATILYEILLGKATLYAVLV |
| | | SALVLMAMVKRKDF |
| 218 | R11P3D3KE | MEKNPLAAPLLILWFHLDCVSSILNVEQSPQSLHVQEGD |
| | alpha chain | STNFTCSFPSSNFYALHWYRKETAKSPEALFVMTLNGD |
| | | EKKKGRISATLNTKEGYSYLYIKGSQPEDSATYLCALYN |
| | | NNDMRFGAGTRLTVKPNIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 219 | R11P3D3KE beta | NNNVPIDDSGMPEDRFSAKMPNASFSTLKIQPSEPRDSA |
| | chain | VYFCASSPGSTDTQYFGPGTRLTVLEDLKNVFPPEVAVF |
| | | EPSEAEISHTQKATLVCLATGFYPDHVELSWWVNGKEV |
| | | HSGVSTDPQPLKEQPALNDSRYCLSSRLRVSATFWQNP |
| | | RNHFRCQVQFYGLSENDEWTQDRAKPVTQIVSAEAWG |
| | | RADCGFTSESYQQGVLSAT1LYEILLGKATLYAVLVSAL |
| | | VLMAMVKRKDSRG |
| 220 | R39P1C12 alpha | TYLYWYKQEPGAGLQLLTYIFSNMDMKQDQRLTVLLN |
| | chain | KKDKHLSLRIADTQTGDSAIYFCAEIDNQGGKLIFGQGT |
| | | ELSVKPNIQNPDPAVYQLRDSKSSDKSVCLFTDFDSQTN |
| | | VSQSKDSDVYITDKTVLDMRSMDFKSNSAVAWSNKSD |
| | | FACANAFNNSIIPEDTFFPSPESSCDVKLVEKSFETDTNL |
| | | NFQNLSVIGFRILLLKVAGFNLLMTLRLWSS |
| 221 | R39P1C12 beta | MGPGLLCWALLCLLGAGLVDAGVTQSPTHLIKTRGQQ |
| | chain | VTLRCSPKSGHDTVSWYQQALGQGPQFIFQYYEEEERQ |
| | | RGNFPDRFSGHQFPNYSSELNVNALLLGDSALYLCASS |
| | | QLNTEAFFGQGTRLTVVEDLNKVFPPEVAVFEPSEAEIS |
| | | HTQKATLVCLATGFFPDHVELSWWVNGKEVHSGVSTD |
| | | PQPLKEQPALNDSRYCLSSRLRVSATFWQNPRNHFRCQ |
| | | VQFYGLSENDEWTQDRAKPVTQIVSAEAWGRADCGFT |
| | | SVSYQQGVLSATILYEILLGKATLYAVLVSALVLMAMV |
| | | KRKDF |
| 222 | R39P1F5 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVNN |
| | | ARLMFGDGTQLVVKPNIQNPDPAVYQLRDSKSSDKSVC |
| | | LFTDFDSQTNVSQSKDSDVYITDKTVLDMRSMDFKSNS |
| | | AVAWSNKSDFACANAFNNSIIPEDTFFPSPESSCDVKLV |
| | | EKSFETDTNLNFQNLSVIGFRILLLKVAGFNLLMTLRLW |
| | | SS |
| 223 | R39P1F5 beta | MDTWLVCWAIFSLLKAGLTEPEVTQTPSHQVTQMGQE |
| | chain | VILRCVPISNHLYFYWYRQILGQKVEFLVSFYNNEISEKS |
| | | EIFDDQFSVERPDGSNFTLKIRSTKLEDSAMYFCASSGQ |
| | | GANEQYFGPGTRLTVTEDLKNVFPPEVAVFEPSEAEISH |
| | | TQKATLVCLATGFYPDHVELSWWVNGKEVHSGVSTDP |
| | | QPLKEQPALNDSRYCLSSRLRVSATFWQNPRNHFRCQV |
| | | QFYGLSENDEWTQDRAKPVTQIVSAEAWGRADCGFTS |
| | | ESYQQGVLSATILYEILLGKATLYAVLVSALVLMAMVK |
| | | RKDSRG |
| 224 | R40P1C2 alpha | MACPGFLWALVISTCLEFSMAQTVTQSQPEMSVQEAET |
| | chain | VTLSCTYDTSESDYYLFWYKQPPSRQMILVIRQEAYKQ |
| | | QNATENRFSVNFQKAAKSFSLKISDSQLGDAAMYFCAY |
| | | LNYQLIWGAGTKLIIKPDIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 225 | R40P1C2 beta | MDTWLVCWAIFSLLKAGLTEPEVTQTPSHQVTQMGQE |
| | chain | VILRCVPISNHLYFYWYRQILGQKVEFLVSFYNNEISEKS |
| | | EIFDDQFSVERPDGSNFTLKIRSTKLEDSAMYFCASSEM |
| | | TAVGQYFGPGTRLTVTEDLKNVFPPEVAVFEPSEAEISH |
| | | TQKATLVCLATGFYPDHVELSWWVNGKEVHSGVSTDP |
| | | QPLKEQPALNDSRYCLSSRLRVSATFWQNPRNHFRCQV |
| | | QFYGLSENDEWTQDRAKPVTQIVSAEAWGRADCGFTS |
| | | ESYQQGVLSATILYEILLGKATLYAVLVSALVLMAMVK |
| | | RKDSRG |
| 226 | R41P3E6 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAAFSG |
| | | YALNFGKGTSLLVTPHIQNPDPAVYQLRDSKSSDKSVC |
| | | LFTDFDSQTNVSQSKDSDVYITDKTVLDMRSMDFKSNS |
| | | AVAWSNKSDFACANAFNNSIIPEDTFFPSPESSCDVKLV |
| | | EKSFETDTNLNFQNLSVIGFRILLLKVAGFNLLMTLRLW |
| | | SS |
| 227 | R41P3E6 beta | MDTWLVCWAIFSLLKAGLTEPEVTQTPSHQVTQMGQE |
| | chain | VILRCVPISNHLYFYWYRQILGQKVEFLVSFYNNEISEKS |
| | | EIFDDQFSVERPDGSNFTLKIRSTKLEDSAMYFCASSQY |
| | | TGELFFGEGSRLTVLEDLKNVFPPEVAVFEPSEAEISHTQ |
| | | KATLVCLATGFYPDHVELSWWVNGKEVHSGVSTDPQP |
| | | LKEQPALNDSRYCLSSRLRVSATFWQNPRNHFRCQVQF |
| | | YGLSENDEWTQDRAKPVTQIVSAEAWGRADCGFTSES |
| | | YQQGVLSATILYEILLGKATLYAVLVSALVLMAMVKR |
| | | KDSRG |
| 228 | R43P3G4 alpha | MKSLRVLLVILWLQLSWVWSQQKEVEQNSGPLSVPEG |
| | chain | AIASLNCTYSDRGSQSFFWYRQYSGKSPELIMFIYSNGD |
| | | KEDGRFTAQLNKASQYVSLLIRDSQPSDSATYLCAVNG |
| | | GDMRFGAGTRLTVKPNIQNPDPAVYQLRDSKSSDKSVC |
| | | LFTDFDSQTNVSQSKDSDVYITDKTVLDMRSMDFKSNS |
| | | AVAWSNKSDFACANAFNNSIIPEDTFFPSPESSCDVKLV |
| | | EKSFETDTNLNFQNLSVIGFRILLLKVAGFNLLMTLRLW |
| | | SS |
| 229 | R43P3G4 beta | MDTWLVCWAIFSLLKAGLTEPEVTQTPSHQVTQMGQE |
| | chain | VILRCVPISNHLYFYWYRQILGQKVEFLVSFYNNEISEKS |
| | | EIFDDQFSVERPDGSNFTLKIRSTKLEDSAMYFCASSGQ |
| | | GALEQYFGPGTRLTVTEDLKNVFPPEVAVFEPSEAEISH |
| | | TQKATLVCLATGFYPDHVELSWWVNGKEVHSGVSTDP |
| | | QPLKEQPALNDSRYCLSSRLRVSATFWQNPRNHFRCQV |
| | | QFYGLSENDEWTQDRAKPVTQIVSAEAWGRADCGFTS |
| | | ESYQQGVLSATILYEILLGKATLYAVLVSALVLMAMVK |
| | | RKDSRG |
| 230 | R44P3B3 alpha | MAMLLGASVLILWLQPDWVNSQQKNDDQQVKQNSPS |
| | chain | LSVQEGRISILNCDYTNSMFDYFLWYKKYPAEGPTFLISI |
| | | SSIKDKNEDGRFTVFLNKSAKHLSLHIVPSQPGDSAVYF |
| | | CAASGLYNQGGKLIFGQGTELSVKPNIQNPDPAVYQLR |
| | | DSKSSDKSVCLFTDFDSQTNVSQSKDSDVYITDKTVLD |
| | | MRSMDFKSNSAVAWSNKSDFACANAFNNSIIPEDTFFPS |
| | | PESSCDVKLVEKSFETDTNLNFQNLSVIGFRILLLKVAGF |
| | | NLLMTLRLWSS |
| 231 | R44P3B3 beta | MGCRLLCCVVFCLLQAGPLDTAVSQTPKYLVTQMGND |
| | chain | KSIKCEQNLGHDTMYWYKQDSKKFLKIMFSYNNKELII |
| | | NETVPNRFSPKSPDKAHLNLHINSLELGDSAVYFCASSL |
| | | GDRGYEQYFGPGTRLTVTEDLKNVFPPEVAVFEPSEAEI |
| | | SHTQKATLVCLATGFYPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCGF |
| | | TSESYQQGVLSATILYEILLGKATLYAVLVSALVLMAM |
| | | VKRKDSRG |
| 232 | R44P3E7 alpha | MKTFAGFSFLFLWLQLDCMSRGEDVEQSLFLSVREGDS |
| | chain | SVINCTYTDSSSTYLYWYKQEPGAGLQLLTYIFSNMDM |
| | | KQDQRLTVLLNKKDKHLSLRIADTQTGDSAIYFCAEINN |
| | | NARLMFGDGTQLVVKPNIQNPDPAVYQLRDSKSSDKSV |
| | | CLFTDFDSQTNVSQSKDSDVYITDKTVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 233 | R44P3E7 beta | MLSPDLPDSAWNTRLLCHVMLCLLGAVSVAAGVIQSPR |
| | chain | HLIKEKRETATLKCYPIPRHDTVYWYQQGPGQDPQFLIS |
| | | FYEKMQSDKGSIPDRFSAQQFSDYHSELNMSSLELGDS |
| | | ALYFCASSPPDQNTQYFGPGTRLTVLEDLKNVFPPEVA |
| | | VFEPSEAEISHTQKATLVCLATGFYPDHVELSWWVNGK |
| | | EVHSGVSTDPQPLKEQPALNDSRYCLSSRLRVSATFWQ |
| | | NPRNHFRCQVQFYGLSENDEWTQDRAKPVTQIVSAEA |
| | | WGRADCGFTSESYQQGVLSATILYEILLGKATLYAVLV |
| | | SALVLMAMVKRKDSRG |
| 234 | R49P2B7 alpha | MLLLLVPVLEVIFTLGGTRAQSVTQLGSHVSVSEGALVL |
| | chain | LRCNYSSSVPPYLFWYVQYPNQGLQLLLKYTTGATLVK |
| | | GINGFEAEFKKSETSFHLTKPSAHMSDAAEYFCAVRIFG |
| | | NEKLTFGTGTRLTIIPNIQNPDPAVYQLRDSKSSDKSVCL |
| | | FTDFDSQTNVSQSKDSDVYITDKTVLDMRSMDFKSNSA |
| | | VAWSNKSDFACANAFNNSIIPEDTFFPSPESSCDVKLVE |
| | | KSFETDTNLNFQNLSVIGFRILLLKVAGFNLLMTLRLWS |
| | | S |
| 235 | R49P2B7 beta | MGIRLLCRVAFCFLAVGLVDVKVTQSSRYLVKRTGEKV |
| | chain | FLECVQDMDHENMFWYRQDPGLGLRLIYFSYDVKMKE |
| | | KGDIPEGYSVSREKKERFSLILESASTNQTSMYLCASSL |
| | | MGELTGELFFGEGSRLTVLEDLKNVFPPEVAVFEPSEAE |
| | | ISHTQKATLVCLATGFYPDHVELSWWVNGKEVHSGVS |
| | | TDPQPLKEQPALNDSRYCLSSRLRVSATFWQNPRNHFR |
| | | CQVQFYGLSENDEWTQDRAKPVTQIVSAEAWGRADCG |
| | | FTSESYQQGVLSATILYEILLGKATLYAVLVSALVLMA |
| | | MVKRKDSRG |
| 236 | R55P1G7 alpha | MMKSLRVLLVILWLQLSWVWSQQKEVEQDPGPLSVPE |
| | chain | GAIVSLNCTYSNSAFQYFMWYRQYSRKGPELLMYTYSS |
| | | GNKEDGRFTAQVDKSSKYISLFIRDSQPSDSATYLCAM |
| | | MGDTGTASKLTFGTGTRLQVTLDIQNPDPAVYQLRDSK |
| | | SSDKSVCLFTDFDSQTNVSQSKDSDVYITDKTVLDMRS |
| | | MDFKSNSAVAWSNKSDFACANAFNNSIIPEDTFFPSPES |
| | | SCDVKLVEKSFETDTNLNFQNLSVIGFRILLLKVAGFNL |
| | | LMTLRLWSS |
| 237 | R55P1G7 beta | MGIRLLCRVAFCFLAVGLVDVKVTQSSRYLVKRTGEKV |
| | chain | FLECVQDMDHENMFWYRQDPGLGLRLIYFSYDVKMKE |
| | | KGDIPEGYSVSREKKERFSLILESASTNQTSMYLCASSFG |
| | | GYEQYFGPGTRLTVTEDLKNVFPPEVAVFEPSEAEISHT |
| | | QKATLVCLATGFYPDHVELSWWVNGKEVHSGVSTDPQ |
| | | PLKEQPALNDSRYCLSSRLRVSATFWQNPRNHFRCQVQ |
| | | FYGLSENDEWTQDRAKPVTQIVSAEAWGRADCGFTSES |
| | | YQQGVLSATILYEILLGKATLYAVLVSALVLMAMVKR |
| | | KDSRG |
| 238 | R59P2A7 alpha | MKSLRVLLVILWLQLSWVWSQQKEVEQNSGPLSVPEG |
| | chain | AIASLNCTYSDRGSQSFFWYRQYSGKSPELIMSIYSNGD |
| | | KEDGRFTAQLNKASQYVSLLIRDSQPSDSATYLCAVQP |
| | | HDMRFGAGTRLTVKPNIQNPDPAVYQLRDSKSSDKSVC |
| | | LFTDFDSQTNVSQSKDSDVYITDKTVLDMRSMDFKSNS |
| | | AVAWSNKSDFACANAFNNSIIPEDTFFPSPESSCDVKLV |
| | | EKSFETDTNLNFQNLSVIGFRILLLKVAGFNLLMTLRLW |
| | | SS |
| 239 | R59P2A7 beta | MLCSLLALLLGTFFGVRSQTIHQWPATLVQPVGSPLSLE |
| | chain | CTVEGTSNPNLYWYRQAAGRGLQLLFYSVGIGQISSEV |
| | | PQNLSASRPQDRQFILSSKKLLLSDSGFYLCAWSGLVAE |
| | | QFFGPGTRLTVLEDLKNVFPPEVAVFEPSEAEISHTQKA |
| | | TLVCLATGFYPDHVELSWWVNGKEVHSGVSTDPQPLK |
| | | EQPALNDSRYCLSSRLRVSATFWQNPRNHFRCQVQFYG |
| | | LSENDEWTQDRAKPVTQIVSAEAWGRADCGFTSESYQ |
| | | QGVLSATILYEILLGKATLYAVLVSALVLMAMVKRKDS |
| | | RG |

The term “cytotoxic T lymphocyte” (CTL) as used herein refers to a T lymphocyte that expresses CD8 on the surface thereof (i.e., a CD8+ T cell). In some embodiments such cells are preferably “memory” T cells (T_{M} cells) that are antigen-experienced.

The term “genetically modified” as used herein includes methods to introduce exogenous nucleic acids into a cell, whether or not the exogenous nucleic acids are integrated into the genome of the cell.

The term “genetically modified cell” as used herein includes cells that contain exogenous nucleic acids whether or not the exogenous nucleic acids are integrated into the genome of the cell.

“Treatment,” “treating,” and the like, as used herein refer broadly to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, e.g., arresting its development; and (c) relieving the disease, e.g., causing regression of the disease.

### Isolation of CD8+ T Cells

Since CD8+ T cells have relatively simple functions as compared with other cells, such as dendritic cells, CD4+ T cells, and NK cells, it is less likely for CD8+ T cells to cause unexpected side effects during anticancer immunotherapy. Generally, antigen-specific CD8+ T cells may be isolated by using MHC class I/peptide multimer, which, however, may stimulate a T cell receptor (TCR). As such, this method may have some drawbacks including high cell death rate caused by cell apoptosis after cell isolation and a long period of culturing time required to produce sufficient amounts of antigen-specific CD8+ T cells.

CD8+ T cells may be isolated from preparations of peripheral blood mononuclear cells (PBMCs) by positive or negative selection, or both. Positive selection may result in a highly-purified population of CD8+ cells. Negative selection, e.g., depleting CD4+ cells, while resulting in sufficient numbers of CD8+ cells, may have low levels of contaminating non-CD8+ populations remaining after the selection procedure. CD8+ T cells may be isolated from preparations of PBMCs using, e.g., anti-CD8 antibodies, which may have high affinity for CD8+ cells, may not activate the cells during the selection process, and may be capable of being easily eluted from the cells. Anti-CD8 antibodies are known in the art and are commercially available.

In another embodiment of the present disclosure, CD8+ cells may be CD8+CD62L+ T cells, which may be isolated using a two-step procedure. After depletion of non-CD8+ cells, e.g., CD4+ T cells, monocytes, neutrophils, eosinophils, B cells, stem cells, dendritic cells, NK cells, granulocytes, γ/δ T cells, or erythroid cells, which may be labeled by using a cocktail of biotin-conjugated antibodies that may contain antibodies against, e.g., CD4, CD15, CD16, CD19, CD34, CD3δ, CD56, CD123, TCRγ/δ, and/or CD235a (Glycophorin A), the CD8+CD62L+ T cells may be positively isolated using CD62L microbeads. The magnetically labeled CD8+CD62L+ T cells may be retained within the column, e.g., MACS column (Miltenyi Biotec), and eluted after removal of the column from the magnetic field. The CD8+ T cells are collected, and, optionally, stored, until used in a method described herein for the production of genetically modified CD8+ T cells.

### T Cell Activation

The CD8+ selected T cells may be activated, wherein the T cells that have been sufficiently stimulated to induce detectable cellular proliferation. Activation can also be associated with induced cytokine production, and detectable effector functions. Signals generated through the TCR alone are insufficient for full activation of the T cell and one or more secondary or costimulatory signals are also required. Thus, T cell activation comprises a primary stimulation signal through the TCR/CD3 complex and one or more secondary costimulatory signals. Co-stimulation can be evidenced by proliferation and/or cytokine production by T cells that have received a primary activation signal, such as stimulation through the CD3/TCR complex or through CD2.

A population of T cells may be induced to proliferate by activating T cells and stimulating an accessory molecule on the surface of T cells with a ligand, which binds the accessory molecule. Activation of a population of T cells may be accomplished by contacting T cells with a first agent which stimulates a TCR/CD3 complex-associated signal in the T cells. Stimulation of the TCR/CD3 complex-associated signal in a T cell may be accomplished either by ligation of the T cell receptor (TCR)/CD3 complex or the CD2 surface protein, or by directly stimulating receptor-coupled signaling pathways. Thus, an anti-CD3 antibody, an anti-CD2 antibody, or a protein kinase C activator in conjunction with a calcium ionophore may be used to activate a population of T cells. Both anti-CD3 and anti-CD2 antibodies are known in the art and are commercially available.

To induce proliferation, an activated population of T cells may be contacted with a second agent, which stimulates an accessory molecule on the surface of the T cells. For example, a population of CD4+ T cells can be stimulated to proliferate with an anti-CD28 antibody directed to the CD28 molecule on the surface of the T cells. Anti-CD28 antibodies are known in the art and are commercially available.

Alternatively, CD4+ T cells can be stimulated with a natural ligand for CD28, such as B7-1 and B7-2. The natural ligand can be soluble, on a cell membrane, or coupled to a solid phase surface. Proliferation of a population of CD8+ T cells may be accomplished by use of a monoclonal antibody ES5.2D8, which binds to CD9, an accessory molecule having a molecular weight of about 27 kD present on activated T cells. Alternatively, proliferation of an activated population of T cells can be induced by stimulation of one or more intracellular signals, which result from ligation of an accessory molecule, such as CD28.

The T cells may be activated in the presence of a cytokine, for example, an interferon alpha (IFN-α), interleukin-2 (IL-2), interleukin-4 (IL-4), interleukin-7 (IL-7), interleukin-9 (IL-9), interleukin-12 (IL-12), interleukin-15 (IL-15), interleukin-21 (IL-21), macrophage colony-stimulating factor (MCSF), interleukin-6 (IL-6), eotaxin-1/CCL11, interferon gamma induced protein 10 (IP-10), IL-RA, macrophage inflammatory protein 1 alpha (MIP-1 □), macrophage inflammatory protein 1 beta (MIP-1 □), interleukin 13 (IL-13), IL-2R, or a combination thereof. The T cells may be activated in the presence of IL-2, preferably human IL-2, more preferably recombinant human IL-2 (rhlL-2). The cytokine may be present in a concentration of about 50 to 150 U/mL, about 50 to about 100 U/mL, or about 100 U/mL.

The agent providing the primary activation signal and the agent providing the costimulatory agent can be added either in soluble form or coupled to a solid phase surface. In a preferred embodiment, the two agents may be coupled to the same solid phase surface.

Following activation and stimulation of an accessory molecule on the surface of the T cells, the progress of proliferation of the T cells in response to continuing exposure to the ligand or other agent, which acts intracellularly to simulate a pathway mediated by the accessory molecule, may be monitored. When the rate of T cell proliferation decreases, T cells may be reactivated and re-stimulated, such as with additional anti-CD3 antibody and a co-stimulatory ligand, to induce further proliferation. The rate of T cell proliferation may be monitored by examining cell size. Alternatively, T cell proliferation may be monitored by assaying for expression of cell surface molecules in response to exposure to the ligand or other agent, such as B7-1 or B7-2. The monitoring and re-stimulation of T cells can be repeated for sustained proliferation to produce a population of T cells increased in number from about 100- to about 100,000-fold over the original T cell population.

The anti-CD3 antibody and the anti-CD28 antibody each may have a concentration of no more than about 0.1 μg/ml, no more than about 0.2 μg/ml, no more than about 0.3 μg/ml, no more than about 0.4 μg/ml, no more than about 0.5 μg/ml, no more than about 0.6 μg/ml, no more than about 0.7 μg/ml, no more than about 0.8 μg/ml, no more than about 0.9 μg/ml, no more than about 1.0 μg/ml, no more than about 2.0 μg/ml, no more than about 4.0 μg/ml, no more than about 6.0 μg/ml, no more than about 8.0 μg/ml, or no more than about 10.0 μg/ml.

The anti-CD3 antibody and the anti-CD28 antibody each may have a concentration of from about 0.1 μg/ml to about 1.0 μg/ml, about 0.1 μg/ml to about 0.8 μg/ml, about 0.1 μg/ml to about 0.6 μg/ml, about 0.1 μg/ml to about 0.5 μg/ml, about 0.1 μg/ml to about 0.25 μg/ml, about 0.2 μg/ml to about 0.5 μg/ml, about 0.2 μg/ml to about 0.3 μg/ml, about 0.3 μg/ml to about 0.5 μg/ml, about 0.3 μg/ml to about 0.4 μg/ml, about 0.2 μg/ml to about 0.5 μg/ml, about 0.1 μg/ml to about 10.0 μg/ml, about 0.1 μg/ml to about 8.0 μg/ml, about 0.1 μg/ml to about 6.0 μg/ml, about 0.1 μg/ml to about 4.0 μg/ml, or about 0.1 μg/ml to about 2.0 μg/ml.

The anti-CD3 antibody and the anti-CD28 antibody may be immobilized on a solid phase support. The solid phase support may be in the form of a bead, box, column, cylinder, disc, dish (e.g., glass dish, PETRI dish), fibre, film, filter, microtiter plate (e.g., 96-well microtiter plate), multi-bladed stick, net, pellet, plate, ring, rod, roll, sheet, slide, stick, tray, tube, or vial. The solid phase support can be a singular discrete body (e.g., a single tube, a single bead), any number of a plurality of substrate bodies (e.g., a rack of 10 tubes, several beads), or combinations thereof (e.g., a tray comprises a plurality of microtiter plates, a column filled with beads, a microtiter plate filed with beads). Conti et al. (2003) Current Protocols in Cytometry John Wiley & Sons, Inc. The solid phase support may be a surface of a bead, tube, tank, tray, dish, a plate, a flask, or a bag. The solid phase support may be an array.

The activation of the CD8+ T cells may be carried for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours. The activation of the T cells may be carried for about 1-10 hours, 11-30 hours, 15-25 hours, 31-50 hours, 51-100 hours, or 101-120 hours.

The activation of the CD8+ T cells may be conducted at a temperature between about 0° C. and about 42° C. The activation of the CD8+ T cells may be conducted at a temperature at about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., or 41° C. The activation of the CD8+ T cells may be conducted at a temperature between about 30° C. and about 40° C.

Conventional methods of activating T cells may involve an open-system and a labor-intensive process using either commercially available beads or non-tissue culture treated 24-well or 6-well plates coated with anti-CD3 and anti-CD28 antibodies (“plate-bound”) at a concentration of 1 ug/mL each. Open system methods, however, may take a relatively long time, e.g., about 8 hours, to complete. To simplify the open-system and the labor-intensive process, the inventors streamlined the system to a process adaptable to a closed-system that can be combined with containers, e.g., bags, of commercially available closed system, e.g., G-Rex® (cell expansion) system and^{Xuri}® cell expansion system, resulting in comparable T cell activation profile, transducibility of T cells, and functionality of the end-product with that of T cells activated using the conventional methods. In addition, methods of the present disclosure, e.g., flask-bound method, may take a relatively short time, e.g., about 1 hour, to complete, which is about 8 times faster than the conventional methods.

The closed system may be CliniMACS Prodigy® (closed and automated platform for cell manufacturing), WAVE (XURI®) Bioreactor (cell expansion system), WAVE (XURI®) Bioreactor (cell expansion system) in combination with BioSafe Sepax® II (cell separation system), G-Rex® closed system (cell expansion system), or G-Rex® closed system (cell expansion system) in combination with BioSafe Sepax® II (cell separation system).

### T Cell Transformation

Nucleic acids encoding recombinant proteins, e.g., CARs, TCRs, cytokines, antibodies, and/or bi-specific binding molecules, may be introduced into the CD8+ T cells as naked DNA or in a suitable vector, such as a viral vector. Methods of stably transfecting T cells by electroporation or other non-viral gene transfer (such as, but not limited to, sonoporation) using naked DNA are known in the art. See, e.g., U.S. Pat. No. 6,410,319; T Cell Protocols (2^{nd} Edition) De Libero (Ed.) 2009 Humana Press; Molecular Cloning: A Laboratory Manual (4^{th} Edition) Green & Sambrook (Ed.) 2012 Cold Spring Harbor Press. Naked DNA generally refers to the DNA encoding recombinant proteins contained in a plasmid expression vector in proper orientation for expression. Advantageously, the use of naked DNA reduces the time required to produce T cells expressing the recombinant proteins.

A viral vector (e.g., a retroviral vector, adenoviral vector, adeno-associated viral vector, or lentiviral vector) can be used to introduce nucleic acids encoding recombinant proteins into the CD8+ T cells. Suitable vectors for use in accordance with the method of the present disclosure are non-replicating in the subject's T cells. A large number of vectors are known that are based on viruses, where the copy number of the virus maintained in the cell is low enough to maintain the viability of the cell. Illustrative vectors that may be used in the methods described herein include the pFB-neo vectors (STRATAGENE®) as well as vectors based on gamma-retrovirus, lentivirus (LV), e.g., human immunodeficiency virus (HIV), simian vacuolating virus 40 (SV40), Epstein-Barr virus (EBV), herpes simplex virus (HSV), or bovine papillomaviruses (BPV). Methods and materials for stably transfecting T cells with viral vectors are known in the art. Viral Vectors for Gene Therapy: Methods and Protocols Machida (Ed.) 2003 Humana Press. See, e.g., T Cell Protocols (2^{nd} Edition) De Libero (Ed.) 2009 Humana Press; Molecular Cloning: A Laboratory Manual (4^{th} Edition) Green & Sambrook (Ed.) 2012 Cold Spring Harbor Press.

In an aspect, viruses may refer to natural occurring viruses as well as artificial viruses. Viruses in accordance to some embodiments of the present disclosure may be either an enveloped or non-enveloped virus. Parvoviruses (such as AAVs) are examples of non-enveloped viruses. In a preferred embodiment, the viruses may be enveloped viruses. In preferred embodiments, the viruses may be retroviruses and in particular lentiviruses. Viral envelope proteins that can promote viral infection of eukaryotic cells may include HIV-1 derived lentiviral vectors (LVs) pseudotyped with envelope glycoproteins (GPs) from the vesicular stomatitis virus (VSV-G), the modified feline endogenous retrovirus (RD114TR), and the modified gibbon ape leukemia virus (GALVTR). These envelope proteins can efficiently promote entry of other viruses, such as parvoviruses, including adeno-associated viruses (AAV), thereby demonstrating their broad efficiency. For example, other viral envelop proteins may be used including Moloney murine leukemia virus (MLV) 4070 env (such as described in Merten et al., J. Virol. 79:834-840, 2005; the content of which is incorporated herein by reference), RD114 env, chimeric envelope protein RD114pro or RDpro (which is an RD114-HIV chimera that was constructed by replacing the R peptide cleavage sequence of RD114 with the HIV-1 matrix/capsid (MA/CA) cleavage sequence, such as described in Bell et al. Experimental Biology and Medicine 2010; 235: 1269-1276; the content of which is incorporated herein by reference), baculovirus GP64 env (such as described in Wang et al. J. Virol. 81:10869-10878, 2007; the content of which is incorporated herein by reference), or GALV env (such as described in Merten et al., J. Virol. 79:834-840, 2005; the content of which is incorporated herein by reference), or derivatives thereof.

The term “statin,” “vastatin,” or as used interchangeably herein “3-hydroxy-3-methylglutaryl coenzyme A (HMG-CoA) reductase inhibitor” refers to a pharmaceutical agent which inhibits the enzyme 3-hydroxy-3-methylglutaryl-coenzyme A (HMG-CoA) reductase. This enzyme is involved in the conversion of HMG-CoA to mevalonate, which is one of the steps in cholesterol biosynthesis. Such inhibition is readily determined according to standard assays well known to those skilled in the art.

Representative statins which may be used in accordance with this present disclosure include atorvastatin, disclosed in U.S. Pat. No. 4,681,893; atorvastatin calcium, disclosed in U.S. Pat. No. 5,273,995; cerivastatin, disclosed in U.S. Pat. No. 5,502,199; dalvastatin, disclosed in U.S. Pat. No. 5,316,765; fluindostatin, disclosed in U.S. Pat. No. 4,915,954; fluvastatin, disclosed in U.S. Pat. No. 4,739,073; lovastatin, disclosed in U.S. Pat. No. 4,231,938; mevastatin, disclosed in U.S. Pat. No. 3,983,140; pravastatin, disclosed in U.S. Pat. No. 4,346,227; simvastatin, disclosed in U.S. Pat. No. 4,444,784; velostatin, disclosed in U.S. Pat. Nos. 4,448,784 and 4,450,171; and rosuvastatin, disclosed in U.S. Pat. Nos. 6,858,618 and 7,511,140. Each of these references are hereby incorporated by reference in their entireties. Preferred 3-hydroxy-3-methylglutaryl coenzyme A reductase inhibitors may include atorvastatin, atorvastatin calcium, also known as Liptor®., lovastatin, also known as Mevacor®., pravastatin, also known as Pravachol®., simvastatin, also known as Zocor®, and rosuvastatin.

Because the low-density lipoprotein-receptor (LDLR) is the receptor of vesicular stomatitis virus (VSV) and that statins may increase the LDLR expression, the transduction efficiency of VSV-G pseudotyped lentivirus may be augmented by statins that induced higher LDLR expression. (Gong et al, “Rosuvastatin Enhances VSV-G Lentiviral Transduction of NK Cells via Upregulation of the Low-Density Lipoprotein Receptor,” Mol Ther Methods Clin Dev. (2020) 17: 634-646; the content of which is hereby incorporated by reference in its entirety).

In an aspect, the present disclosure may include a method of preparing T cells for immunotherapy, including (a) isolating CD8+ T cells from peripheral blood mononuclear cells (PBMC), (b) activating the isolated CD8+ T cells in the presence of a statin, (c) transducing the activated T cells with a viral vector pseudotyped with an envelope protein of VSV-G into the activated CD8+ T cells, (d) expanding the transformed CD8+ T cells, and (e) harvesting the expanded T CD8+ cells, in which the total time to complete steps (b), (c), (d), and (e) is from about 6 days to about to about 10 days, about 6 days, about 7 days, about 8 days, about 9 days, or about 10 days.

Once it is established that the transfected or transduced T cell is capable of expressing the recombinant proteins, e.g., CARs and TCRs, as surface membrane proteins with the desired regulation and at a desired level, it can be determined whether the CARs and TCRs are functional in the host cell to provide for the desired signal induction. Subsequently, the transduced T cells may be reintroduced or administered to the subject to activate anti-tumor responses in the subject.

### T Cell Expansion

Following T cell transduction, the cells may be propagated for days, weeks, or months ex vivo as a bulk population within about 1, 2, 3, 4, 5 days or more following gene transfer into cells. In a further aspect, following transduction, the transduced cells are cloned and a clone demonstrating presence of a single integrated or episomally maintained expression cassette or plasmid, and expression of recombinant proteins, e.g., TCRs, may be expanded ex vivo. The clones selected for expansion demonstrates the capacity to specifically recognize and lyse peptide-expressing target cells. The genetically modified T cells may be expanded by stimulation with IL-2, or other cytokines that bind the common gamma-chain (e.g., IFN-α, IL-4, IL-7, IL-9, IL-12, IL-15, IL-21, and others). The genetically modified T cells may be expanded by stimulation with artificial antigen presenting cells. The genetically modified T cells may be expanded on artificial antigen presenting cell or with an antibody, such as OKT3, which cross links CD3 on the T cell surface. Subsets of the genetically modified T cells may be deleted on artificial antigen presenting cell or with an antibody, such as Campath, which binds CD52 on the T cell surface. The genetically modified T cells may be cryopreserved.

Expansion of the T cells may be carried out in the presence of the T cell activation stimulus.

The expansion of the T cells may be carried out within a period of no more than about 1 day, no more than about 2 days, no more than about 3 days, no more than about 4 days, no more than about 5 days, or no more than about 6 days. The expansion of the T cells may be for about 1, 2, 3, 4, 5, or 6 days.

Expansion of the T cells may be carried out within a period of from about 1 day to about 6 days, about 1 day to about 5 days, about 1 day to about 4 days, about 1 day to about 3 days, about 1 day to about 2 days, or about 1 day.

Expansion of the T cells may be carried out in the presence of interferon (IFN)-α, interleukin (IL)-2, IL-4, IL-7, IL-9, IL-12, IL-15, IL-21, or a combination thereof. In an aspect, the expansion takes place in the presence of a combination IL-7 and IL-15.

Following expansion of the T cell population to sufficient numbers, the expanded T cells may be restored to the individual. The method of the present disclosure may also provide a renewable source of T cells. Thus, T cells from an individual can be expanded ex vivo, a portion of the expanded population can be re-administered to the individual and another portion can be frozen in aliquots for long term preservation, and subsequent expansion and administration to the individual. Similarly, a population of tumor-infiltrating lymphocytes can be obtained from an individual afflicted with cancer and the T cells stimulated to proliferate to sufficient numbers and restored to the individual.

The present disclosure may also pertain to compositions containing an agent that provides a costimulatory signal to a T cell for T cell expansion (e.g., an anti-CD28 antibody, B7-1 or B7-2 ligand), coupled to a solid phase surface, which may additionally include an agent that provides a primary activation signal to the T cell (e.g., an anti-CD3 antibody) coupled to the same solid phase surface. These agents may be preferably attached to beads or flasks or bags. Compositions comprising each agent coupled to different solid phase surfaces (e.g., an agent that provides a primary T cell activation signal coupled to a first solid phase surface and an agent that provides a costimulatory signal coupled to a second solid phase surface) may also be within the scope of this disclosure.

### Serum Free Medium in T Cell Manufacturing

As referred to herein, the term “serum-free media” or “serum-free culture medium” means that the growth media used is not supplemented with serum (e.g., human serum or bovine serum). In other words, no serum is added to the culture medium as an individually separate and distinct ingredient for the purpose of supporting the viability, activation and grown of the cultured cells. Any suitable culture medium T cell growth media may be used for culturing the cells in accordance with the methods described herein. For example, a T cell growth media may include, but is not limited to, a sterile, low glucose solution that includes a suitable amount of buffer, magnesium, calcium, sodium pyruvate, and sodium bicarbonate. In one embodiment, the T cell growth media may include serum free media, e.g., OPTI-MEM®, D-MEM/F-12, and/or viral production (VP) media (Life Technologies), but one skilled in the art would understand how to generate similar media. In contrast to typical methods for producing engineered T cells, the methods described herein use culture medium that may be not supplemented with serum (e.g., human or bovine).

VSV-G pseudotyped HIV and FIV vectors produced in human cells may be inactivated by human serum complement (DePolo et al. “VSV-G Pseudotyped Lentiviral Vector Particles Produced in Human Cells Are Inactivated by Human Serum,” Molecular Therapy (2000) 2:218-222; the content of which is hereby incorporated by reference in its entirety). In addition, reducing serum concentrations in culture media may result in a more sustainable process with equivalent growth kinetics and product quality (Tyagarajan et al. “Optimizing CAR-T Cell Manufacturing Processes during Pivotal Clinical Trials,” Molecular Therapy: Methods & Clinical Development, (2020) 16:136-144; the content of which is hereby incorporated by reference in its entirety). Therefore, it may be advantageous to include serum free media in T cell manufacturing process.

In an aspect, T cell activation, T cell transformation, and/or T cell expansion may be performed in serum free medium.

In an aspect, T cell activation may be performed in serum free medium or in the presence of serum.

In an aspect, T cell activation may be performed in serum free medium.

In an aspect, T cell transformation may be performed in serum free medium or in the presence of serum.

In an aspect, T cell transformation may be performed in serum free medium.

In an aspect, T cell expansion may be performed in serum free medium or in the presence of serum.

In an aspect, T cell expansion may be performed in serum free medium.

In an aspect, cryopreserved T cells may be thawed and rested in the presence of serum for about 2-8 hours, 2-6, hours, or 2-4 hours.

In an aspect, cryopreserved T cells may be thawed and rested in the presence of serum for about 2-4 hours, activated in the presence of serum, transduced in the absence of serum, and expanded in the presence of serum.

In an aspect, cryopreserved T cells may be thawed and rested in the presence of serum for about 2-4 hours, activated in the absence of serum, transduced in the absence of serum, and expanded in the presence of serum.

### Pharmaceutical Compositions

To facilitate administration, the transduced CD8+ T cells according to the disclosure can be made into a pharmaceutical composition or made into an implant appropriate for administration in vivo, with pharmaceutically acceptable carriers or diluents. The means of making such a composition or an implant are described in the art. See, e.g., Remington's Pharmaceutical Sciences, 16th Ed., Mack, ed. (1980).

The transduced T cells can be formulated into a preparation in semisolid or liquid form, such as a capsule, solution, infusion, or injection. Means known in the art can be utilized to prevent or minimize release and absorption of the composition until it reaches the target tissue or organ, or to ensure timed-release of the composition. Desirably, however, a pharmaceutically acceptable form is employed that does not hinder the cells from expressing the CARs or TCRs. Thus, desirably the transduced T cells can be made into a pharmaceutical composition comprising a carrier. The T cells produced by the methods described herein can be formulated with a physiologically acceptable carrier or excipient to prepare a pharmaceutical composition. The carrier and composition can be sterile. Preferred carriers include, for example, a balanced salt solution, preferably Hanks' balanced salt solution, or normal saline. The formulation should suit the mode of administration. Suitable pharmaceutically acceptable carriers include but are not limited to water, salt solutions (e.g., NaCl), saline, buffered saline, as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, that do not deleteriously react with the T-cells.

A composition of the present invention can be provided in unit dosage form wherein each dosage unit, e.g., an injection, contains a predetermined amount of the composition, alone or in appropriate combination with other active agents.

Compositions may comprise an effective amount of the isolated transduced T cells and be introduced into the subject such that long-term, specific, anti-tumor responses is achieved to reduce the size of a tumor or eliminate tumor growth or regrowth than would otherwise result in the absence of such treatment. For example, the amount of transduced T cells reintroduced into the subject causes an about 10%, about 20%, about 30%, about 40%, about 50%, about 60%, about 70%, about 80%, about 90%, about 95%, about 98%, or about 99% decrease in tumor size when compared to otherwise same conditions where the transduced T cells are not present.

Accordingly, the amount of transduced T cells administered may take into account the route of administration and should be such that a sufficient number of the transduced T cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of transduced T cells desirably should be sufficient to provide in the subject being treated, for example, effective amounts of transduced T cells may be about 1×10⁶ to about 1×10⁹ transduced T cells/m² (or kg) of a patient, even more desirably, from about 1×10⁷ to about 5×10⁸ transduced T cells/m² (or kg) of a patient. Any suitable amount can be utilized, e.g., greater than 5×10⁸ cells/m² (or kg) of a patient, or below, e.g., less than 1×10⁷ cells/m² (or kg) of a patient, as is necessary to achieve a therapeutic effect. The dosing schedule can be based on well-established cell-based therapies (See, e.g., U.S. Pat. No. 4,690,915), or an alternate continuous infusion strategy can be employed.

The T cells may be administered to the patient intravenously. The T cells may be administered to the patient by intravenous infusion. The dosage of T-cells may be between about 0.8-1.2×10⁹ T cells. The dosage of T-cells may be about×10⁹ T cells, about 3-6×10⁹ T cells, about 10×10⁹ T cells, about 5×10⁹ T cells, about 0.1×10⁹ T cells, about 1×10⁸ T cells, about 5×10⁸ T cells, about 1.2-6×10⁹ T cells, about 1-6×10⁹ T cells, or about 1-8×10⁹ ‘1’ cells. The T cells may be administered over the course of 1-3 weeks, preferably about 3 weeks. The T cells may be administered in escalating dosages.

The T-cell products described herein may also be cryopreserved. Accordingly, cryopreserved T-cell compositions may comprise the genetically modified T-cells and a freezing media.

### Pretreatment

The methods described herein may further comprise administering a chemotherapy agent. The dosage of the chemotherapy agent may be sufficient to deplete the patient's T-cell population. The chemotherapy may be administered about 5-7 days prior to T-cell administration. The chemotherapy agent may be cyclophosphamide, fludarabine, or a combination thereof. The chemotherapy agent may comprise dosing at about 400-600 mg/m²/day of cyclophospharnicle. The chemotherapy agent may comprise dosing at about 10-30 mg/m²/day of fludarabine.

The methods described herein may further comprise pre-treatment of the patient with low-dose radiation prior to administration of the composition comprising T-cells. The low dose radiation may comprise about 1.4 Gy for about 1 to about 6 days, about 2 to about 5 days, about 6, about 5, about 6 days, prior to administration of the composition comprising T-cells.

The patient may be HLA-A*02.

The patient may be HLA-A*06.

The methods described herein may further comprise administering an anti-PD1 antibody. The anti-PD1 antibody may be a humanized antibody. The anti-PD1 antibody may be pembrolizumab. The dosage of the anti-PD1 antibody may be about 200 mg. The anti-PD1 antibody may be administered every 3 weeks following T-cell administration.

### Immunotherapy

Methods of treating a patient or individual having a cancer or in need of a treatment thereof, may comprise administering to the patient an effective amount of the expanded genetically modified T cells described herein. The patient or individual in need thereof may be a cancer patient. The cancer to be treated by the T cells descried herein may be hepatocellular carcinoma (HCC), colorectal carcinoma (CRC), glioblastoma (GB), gastric cancer (GC), esophageal cancer, non-small cell lung cancer (NSCLC), pancreatic cancer (PC), renal cell carcinoma (RCC), benign prostate hyperplasia (BPH), prostate cancer (PCA), ovarian cancer (OC), melanoma, breast cancer, chronic lymphocytic leukemia (CLL), Merkel cell carcinoma (MCC), small cell lung cancer (SCLC), Non-Hodgkin lymphoma (NHL), acute myeloid leukemia (AML), gallbladder cancer and cholangiocarcinoma (GBC, CCC), urinary bladder cancer (UBC), acute lymphocytic leukemia (ALL), uterine cancer (UEC), or a combination thereof.

T-cell based immunotherapy targets peptide epitopes derived from tumor-associated or tumor-specific proteins, which are presented by molecules of the major histocompatibility complex (MHC). The antigens that are recognized by the tumor specific T lymphocytes, that is, the epitopes thereof, can be molecules derived from all protein classes, such as enzymes, receptors, transcription factors, etc. which are expressed and, as compared to unaltered cells of the same origin, usually up-regulated in cells of the respective tumor.

There are two classes of MHC-molecules, MHC class I and MHC class II. MHC class I molecules are composed of an alpha heavy chain and beta-2-microglobulin, MHC class II molecules of an alpha and a beta chain. Their three-dimensional conformation results in a binding groove, which is used for non-covalent interaction with peptides. MHC class I molecules can be found on most nucleated cells. They present peptides that result from proteolytic cleavage of predominantly endogenous proteins, defective ribosomal products (DRIPs) and larger peptides. However, peptides derived from endosomal compartments or exogenous sources are also frequently found on MHC class I molecules. This non-classical way of class I presentation is referred to as cross-presentation. MHC class II molecules can be found predominantly on professional antigen presenting cells (APCs), and primarily present peptides of exogenous or transmembrane proteins that are taken up by APCs, e.g., during endocytosis, and are subsequently processed.

Complexes of peptide and MHC class I are recognized by CD8+ T-cells bearing the appropriate T-cell receptor (TCR), whereas complexes of peptide and MHC class II molecules are recognized by CD4-positive-helper-T-cells bearing the appropriate TCR. It is well known that the TCR, the peptide and the MHC are thereby present in a stoichiometric amount of 1:1:1.

CD4+ helper T-cells play an important role in inducing and sustaining effective responses by CD8+ cytotoxic T-cells. The identification of CD4-positive T-cell epitopes derived from tumor associated antigens (TAA) is of great importance for the development of pharmaceutical products for triggering anti-tumor immune responses. At the tumor site, T helper cells, support a cytotoxic T-cell-(CTL-) friendly cytokine milieu and attract effector cells, e.g., CTLs, natural killer (NK) cells, macrophages, and granulocytes.

For an MHC class I peptide to trigger (elicit) a cellular immune response, it also must bind to an MHC-molecule. This process is dependent on the allele of the MHC-molecule and specific polymorphisms of the amino acid sequence of the peptide. MHC-class-1-binding peptides are usually 8-12 amino acid residues in length and usually contain two conserved residues (“anchors”) in their sequence that interact with the corresponding binding groove of the MHC-molecule. In this way, each MHC allele has a “binding motif” determining which peptides can bind specifically to the binding groove.

In the MHC class I dependent immune reaction, peptides not only have to be able to bind to certain MHC class I molecules expressed by tumor cells, they subsequently also have to be recognized by T-cells bearing specific T-cell receptors (TCR).

For proteins to be recognized by T-lymphocytes as tumor-specific or -associated antigens, and to be used in a therapy, particular prerequisites must be fulfilled. The antigen should be expressed mainly by tumor cells and not, or in comparably small amounts, by normal healthy tissues. The peptide should be over-presented by tumor cells as compared to normal healthy tissues. It is furthermore desirable that the respective antigen is not only present in a type of tumor, but also in high concentrations (e.g., copy numbers of the respective peptide per cell). Tumor-specific and tumor-associated antigens are often derived from proteins directly involved in transformation of a normal cell to a tumor cell due to their function, e.g., in cell cycle control or suppression of apoptosis. Additionally, downstream targets of the proteins directly causative for a transformation may be up-regulated and thus may be indirectly tumor-associated. Such indirect tumor-associated antigens may also be targets of a vaccination approach. Epitopes are present in the amino acid sequence of the antigen, in order to ensure that such a peptide (“immunogenic peptide”), being derived from a tumor associated antigen, and leads to an in vitro or in vivo T-cell-response.

TAAs are a starting point for the development of a T-cell based therapy including but not limited to tumor vaccines. The methods for identifying and characterizing the TAAs are usually based on the use of T-cells that can be isolated from patients or healthy subjects, or they are based on the generation of differential transcription profiles or differential peptide expression patterns between tumors and normal tissues. However, the identification of genes over-expressed in tumor tissues or human tumor cell lines, or selectively expressed in such tissues or cell lines, does not provide precise information as to the use of the antigens being transcribed from these genes in an immune therapy. This is because only an individual subpopulation of epitopes of these antigens are suitable for such an application since a T-cell with a corresponding TCR has to be present and the immunological tolerance for this particular epitope needs to be absent or minimal. In a very preferred embodiment of the description it is therefore important to select only those over- or selectively presented peptides against which a functional and/or a proliferating T-cell can be found. Such a functional T-cell is defined as a T-cell, which upon stimulation with a specific antigen can be clonally expanded and is able to execute effector functions (“effector T-cell”).

TAA peptides that are capable of use with the methods and embodiments described herein include, for example, those TAA peptides described in U.S. Patent Application Publication Nos. 2016/0187351; 2017/0165335; 2017/0035807; 2016/0280759; 2016/0287687; 2016/0346371; 2016/0368965; 2017/0022251; 2017/0002055; 2017/0029486; 2017/0037089; 2017/0136108; 2017/0101473; 2017/0096461; 2017/0165337; 2017/0189505; 2017/0173132; 2017/0296640; 2017/0253633; 2017/0260249; 2018/0051080, and 2018/0164315, the contents of each which are incorporated by reference in their entireties.

The T cells described herein selectively recognize cells which present a TAA peptide described in one of more of the patents and publications described above.

TAA that are capable of use with the methods and embodiments described herein include at least one amino acid sequence of SEQ ID NO: 1 to SEQ ID NO: 161. T cells selectively recognize cells which present a TAA peptide described in the amino acid sequences of SEQ ID NO: 1-161 or any of the patents or applications described herein.

TABLE 3
List of Tumor Associated Antigens (TAAs)

| SEQ ID | Amino Acid |
|---|---|
| NO: | Sequence |
| 1 | YLYDSETKNA |
| 2 | HLMDQPLSV |
| 3 | GLLKKINSV |
| 4 | FLVDGSSAL |
| 5 | FLFDGSANLV |
| 6 | FLYKIIDEL |
| 7 | FILDSAETTTL |
| 8 | SVDVSPPKV |
| 9 | VADKIHSV |
| 10 | IVDDLTINL |
| 11 | GLLEELVTV |
| 12 | TLDGAAVNQV |
| 13 | SVLEKEIYSI |
| 14 | LLDPKTIFL |
| 15 | YTFSGDVQL |
| 16 | YLMDDFSSL |
| 17 | KVWSDVTPL |
| 18 | LLWGHPRVALA |
| 19 | KIWEELSVLEV |
| 20 | LLIPFTIFM |
| 21 | FLIENLLAA |
| 22 | LLWGHPRVALA |
| 23 | FLLEREQLL |
| 24 | SLAETIFIV |
| 25 | TLLEGISRA |
| 26 | ILQDGQFLV |
| 27 | VIFEGEPMYL |
| 28 | SLFESLEYL |
| 29 | SLLNQPKAV |
| 30 | GLAEFQENV |
| 31 | KLLAVIHEL |
| 32 | TLHDQVHLL |
| 33 | TLYNPERTITV |
| 34 | KLQEKIQEL |
| 35 | SVLEKEIYSI |
| 36 | RVIDDSLVVGV |
| 37 | VLFGELPAL |
| 38 | GLVDIMVHL |
| 39 | FLNAIETAL |
| 40 | ALLQALMEL |
| 41 | ALSSSQAEV |
| 42 | SLITGQDLLSV |
| 43 | QLIEKNWLL |
| 44 | LLDPKTIFL |
| 45 | RLHDENILL |
| 46 | YTFSGDVQL |
| 47 | GLPSATTTV |
| 48 | GLLPSAESIKL |
| 49 | KTASINQNV |
| 50 | SLLQHLIGL |
| 51 | YLMDDFSSL |
| 52 | LMYPYIYHV |
| 53 | KVWSDVTPL |
| 54 | LLWGHPRVALA |
| 55 | VLDGKVAVV |
| 56 | GLLGKVTSV |
| 57 | KMISAIPTL |
| 58 | GLLETTGLLAT |
| 59 | TLNTLDINL |
| 60 | VIIKGLEEI |
| 61 | YLEDGFAYV |
| 62 | KIWEELSVLEV |
| 63 | LLIPFTIFM |
| 64 | ISLDEVAVSL |
| 65 | KISDFGLATV |
| 66 | KLIGNIHGNEV |
| 67 | ILLSVLHQL |
| 68 | LDSEALLTL |
| 69 | VLQENSSDYQSNL |
| 70 | HLLGEGAFAQV |
| 71 | SLVENIHVL |
| 72 | YTFSGDVQL |
| 73 | SLSEKSPEV |
| 74 | AMFPDTIPRV |
| 75 | FLIENLLAA |
| 76 | FTAEFLEKV |
| 77 | ALYGNVQQV |
| 78 | LFQSRIAGV |
| 79 | ILAEEPIYIRV |
| 80 | FLLEREQLL |
| 81 | LLLPLELSLA |
| 82 | SLAETIFIV |
| 83 | AILNVDEKNQV |
| 84 | RLFEEVLGV |
| 85 | YLDEVAFML |
| 86 | KLIDEDEPLFL |
| 87 | KLFEKSTGL |
| 88 | SLLEVNEASSV |
| 89 | GVYDGREHTV |
| 90 | GLYPVTLVGV |
| 91 | ALLSSVAEA |
| 92 | TLLEGISRA |
| 93 | SLIEESEEL |
| 94 | ALYVQAPTV |
| 95 | KLIYKDLVSV |
| 96 | ILQDGQFLV |
| 97 | SLLDYEVSI |
| 98 | LLGDSSFFL |
| 99 | VIFEGEPMYL |
| 100 | ALSYILPYL |
| 101 | FLFVDPELV |
| 102 | SEWGSPHAAVP |
| 103 | ALSELERVL |
| 104 | SLFESLEYL |
| 105 | KVLEYVIKV |
| 106 | VLLNEILEQV |
| 107 | SLLNQPKAV |
| 108 | KMSELQTYV |
| 109 | ALLEQTGDMSL |
| 110 | VIIKGLEEITV |
| 111 | KQFEGTVEI |
| 112 | KLQEEIPVL |
| 113 | GLAEFQENV |
| 114 | NVAEIVIHI |
| 115 | ALAGIVTNV |
| 116 | NLLIDDKGTIKL |
| 117 | VLMQDSRLYL |
| 118 | KVLEHVVRV |
| 119 | LLWGNLPEI |
| 120 | SLMEKNQSL |
| 121 | KLLAVIHEL |
| 122 | ALGDKFLLRV |
| 123 | FLMKNSDLYGA |
| 124 | KLIDHQGLYL |
| 125 | GPGIFPPPPPQP |
| 126 | ALNESLVEC |
| 127 | GLAALAVHL |
| 128 | LLLEAVWHL |
| 129 | SIIEYLPTL |
| 130 | TLHDQVHLL |
| 131 | SLLMWITQC |
| 132 | FLLDKPQDLSI |
| 133 | YLLDMPLWYL |
| 134 | GLLDCPIFL |
| 135 | VLIEYNFSI |
| 136 | TLYNPERTITV |
| 137 | AVPPPPSSV |
| 138 | KLQEELNKV |
| 139 | KLMDPGSLPPL |
| 140 | ALIVSLPYL |
| 141 | FLLDGSANV |
| 142 | ALDPSGNQLI |
| 143 | ILIKHLVKV |
| 144 | VLLDTILQL |
| 145 | HLIAEIHTA |
| 146 | SMNGGVFAV |
| 147 | MLAEKLLQA |
| 148 | YMLDIFHEV |
| 149 | ALWLPTDSATV |
| 150 | GLASRILDA |
| 151 | ALSVLRLAL |
| 152 | SYVKVLHHL |
| 153 | VYLPKIPSW |
| 154 | NYEDHFPLL |
| 155 | VYIAELEKI |
| 156 | VHFEDTGKTLLF |
| 157 | VLSPFILTL |
| 158 | HLLEGSVGV |
| 159 | ALREEEEGV |
| 160 | KEADPTGHSY |
| 161 | TLDEKVAEL |
| 240 | KIQEILTQV |

Although the invention has been described in some detail by way of illustration and example for purposes of clarity of understanding, it should be understood that certain changes and modifications may be practiced within the scope of the appended claims. Modifications of the above-described modes for carrying out the invention that would be understood in view of the foregoing disclosure or made apparent with routine practice or implementation of the invention to persons of skill in oncology, physiology, immunology, and/or related fields are intended to be within the scope of the following claims.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

### EXAMPLES

### Example 1

### CD8-Derived Versus Pbmc-Derived T Cell Products

The manufacturing and functionality of genetically modified T cell products derived from bulk Peripheral Blood Mononuclear Cells (PBMC) and CD8+ selected T cells as a starting material from healthy human donors or patients, e.g., cancer patients, may be compared. Genetically modified T cell products may be generated from bulk PBMC and CD8+ selected T cells from each donor using a short 6-day manufacturing process at different scales. Leukapheresis products obtained from each donor may be split into 2 halves and one half may be processed for PBMC isolation and the other for CD8+ T cell selection. Both the cell types may be activated with anti-CD3/anti-CD28 antibodies and transduced with the lentivirus (LV) encoding CAR (LV-CAR) or TCR (LV-TCR). Transduced cells may be expanded in the presence of IL-7 and IL-15 and harvested for phenotypic and functional comparison.

Genetically modified T cell products derived from PBMC and CD8+ selected T cells may be phenotypically compared based on the activation marker profile, fold expansion, transduction efficiency, memory phenotype and vector copy number. The functionality of the TCR-transformed-PBMC and TCR-transformed-CD8 products may be assessed based on intracellular cytokine secretion, killing assays (IncuCyte assay and Flow cytometry based serial-killing assay) and extracellular cytokine secretion in response to the target cells. The inventors surprisingly discovered CD8-derived T cell products showed longer longevity, a preferred cytokine profile, and strong anti-tumor cell activity as compared to PBMC-derived T cell products.

### Example 2

### Cd8+ T Cell Selection and Manufacturing of TCR-Transformed T Cell Products

### Methods

Leukapheresis products from healthy blood donors were obtained from Hema Care, North Ridge Calif. Each leukapheresis product was divided into two halves and one half was processed for the PBMC isolation and the other for CD8+ T cell selection for a paired comparison. Manufacturing of genetically modified T cell products from PBMC or CD8+ selected cells was performed at different scales. A brief description of each step performed is described herein.

### PBMC and CD8+ T Cell Preparation

PBMC were isolated from half of the leukapheresis unit via Ficoll using closed and automated Sepax C-pro system and NeatCell C-Pro Software (cell separation protocol software, GE Healthcare Life Sciences) according to the manufacturer's recommendations. The CD8+ T cells were positively selected from the other half of the leukapheresis unit using CliniMACS® CD8 reagent and CliniMACS® Plus instrument (closed and automated platform for cell processing, Miltenyi Biotech) according to the manufacturer's recommendations. The CD8+ T cell purity was assessed by flow cytometry.

### T Cell Activation

The 750-AC or 290-AC bags (Saint-Gobain) were coated with anti-human CD3 (0.5 μg/ml) and anti-human CD28 (0.5 μg/ml) antibodies for 16-24 hours at 4° C. Freshly prepared PBMC or CD8+ selected T cells were placed in the anti-CD3/anti-CD28 coated bags at the concentration of 1×10⁶/ml in the complete TexMACS media (supplemented with 5% human AB serum) without cytokines at 37° C. for 16-20 hours.

### Transduction

Anti-CD3/anti-CD28-activated PBMC or CD8+ selected T cells were harvested and counted after 16-24 hours. Activated PBMC or CD8+ selected T cells were mixed with the transduction cocktail containing; lentivirus encoding a TCR (2.5 μl/10⁶ cells) Protamine sulfate (1 μg/ml), IL-7 (10 ng/ml) and IL-15 (50 ng/ml) in a complete TexMACS media (2×10⁶ cells/ml) in a Grex100 or Grex-500 for 24 hours at 37° C. After 24 hours, transduced cells were fed with the TexMACS media with IL-7 and IL-15 to obtain a final seeding density of 0.5-0.8×10⁶/cm².

### Harvest and Cryopreservation

At day 6, transduced and non-transduced cells were harvested, counted and cryopreserved in the CryoStor CS5 Freeze Media. Phenotypic and functional analysis was performed post thawing of the cryopreserved genetically modified T cell products.

### Flow Cytometry

1.0×10⁶ transduced cells were stained for live-dead fixable dye and Activation panel or Dextramer panel or Memory T cell panel.

TABLE 4
Activation, Dextramer, and Memory T Cell Panel and
Antibodies Used

| Panel | Antibodies used |
|---|---|
| Activation | CD8a-BV421, CD3-AF488, hLDL-R PE, |
| panel | CD69-PerCP Cy5.5, CD25-PE-Cy7, |
| | CD4-APC-Fire750 |
| Dextramer | CD8a-AF488, CD3-PerCP-Cy5.5, |
| panel | Dextramer-PE, CD4-PE-Cy7 |
| Memory T | CD8a-AF488, CD3-PerCP-Cy5.5, |
| cell panel | Dextramer-PE, CD27-PE-Dazzle594, |
| | CD4-PE-Cy7, CD57-APC, CD127-AF700, |
| | CD45RA-APC-Cy7, CCR7-BV421, |
| | CD45RO-BV605, CD28-BV650 and |
| | CD62L-BV785 |
| Serial Killing | CD3-BV421 |
| assay | |
| CD8+ purity | CD3-BV421, CD8a-APC, CD4 PE-Cy7 |
| ICS panel | CD3-PerCP-Cy5.5, CD8-FITC, |
| | CD4-APC-Cy7, VB8-BV605, CD107a-PE, |
| | IL-2-BV421, IFN-γ-AF700, TNF-α-BV786, |
| | Granzyme B-PE-CF594, MIP-1b-PE Cy7 |

For the intracellular staining (ICS), TCR-transformed-PBMC or TCR-transformed-CD8 products were cocultured with UACC257 at 1:1 ratio for 12 hour, 4 hours before the harvest, brefeldin A was added to the cultures. Cells were harvested and surface and intracellular staining was performed according to the manufacturer's recommendations. Samples were acquired with auto-compensation matrix derived from compensation beads.

### Vector Copy Number Determination

The vector copy number of the genetically modified T cell products derived from PBMCs and CD8+ T cells was performed by methods known in the art, for example, Charrier, S., Ferrand, M., Zerbato, M. et al., Gene Ther 18, 479-487 (2011), the content of which is hereby incorporated by reference in its entirety. Alternatively, vector copy number may be determined by isolating DNA from the transduced products derived from PBMC or selected CD8. Using lentivirus psi sequence primers, qPCR may be carried out employing relative quantitation method. Albumin may be used as housekeeping control. Known quantities of plasmid expressing both psi and albumin may be diluted 10-fold to create a standard curve. Vector copy number may be calculated by normalizing psi copies to albumin copies and multiplying with a factor of 2 since there are 2 copies of albumin per genome.

### IncuCyte Cell Killing Assay

PBMC- or CD8-derived genetically modified T cell products (normalized to % CD8+Dex+) were co-cultured with the target cell lines expressing varying levels of target antigen; UACC257-RFP (˜1080 copies), U2-OS-RFP (˜250 copies), A375-RFP (˜50 copies) and MCF7-GFP (0 copy) at 4:1::E:T ratio for 72 hours. The tumor growth was measured. The supernatants from the 24 hour cultures were stored at −80° C. for cytokine analysis.

### Serial Killing Assay

Genetically modified T cell products derived from PBMC or CD8+ selected T cells were co-cultured with the target cell line THP-1-RFP in complete TexMACS without cytokines in a 24-well plate at E:T ratios of 1:1 and 1:5 for 17 days. Every 3-4 days, the residual tumor cells and CD3+ T cells counts were analyzed using FACS counting beads (Thermofisher) according to the manufacturer's recommendations. T cells in the co-cultures were re-challenged with the same number of fresh tumor cells as at the time of initiation and the analysis was repeated every 3 days. Luminex assay

Cytokines released in the 24 hour co-culture supernatants were quantified using Procarta Human cytokine Panel 1A 34 plex immunoassay kit (EPX340-12167-901, Thermofisher) and Human Custom ProcartaPlex16-plex Luminex kit (GM-CSF, Granzyme A/B, IFN-γ, IL-10, IL-13, IL-18, TNF-α, IL-2, IL-4, IL-6, IP-10, MIP-1a and MP-1β, Perforin and RANTES; Thermofisher) according to manufacturer's recommendations.

The CD8+ T cell products showed a desirable cytokine profile and tumor killing capability.

### Comparable Activation Marker Profile

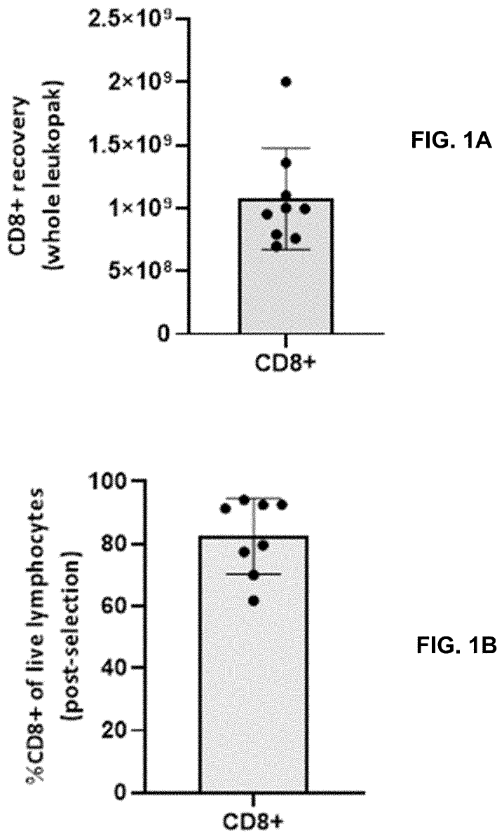

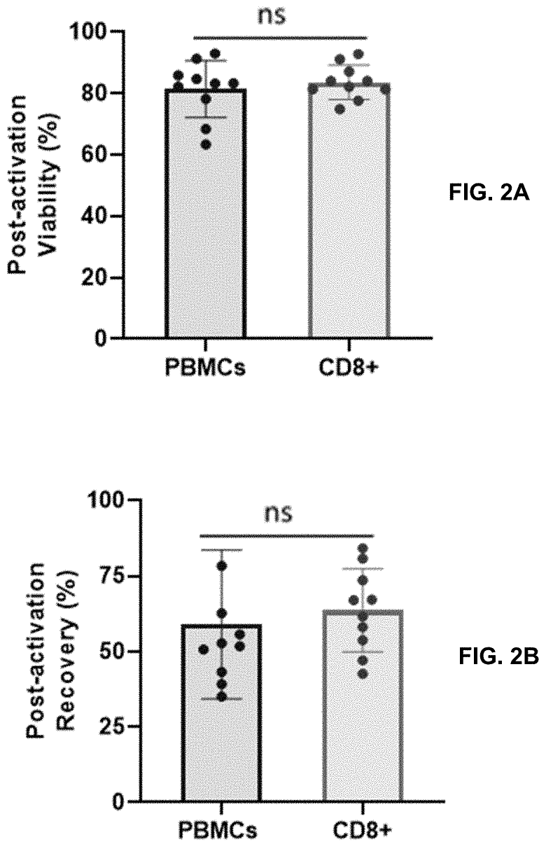

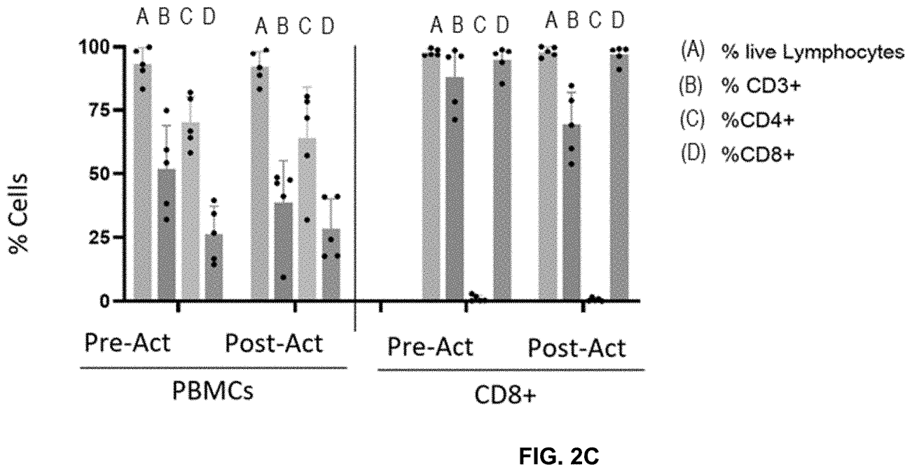

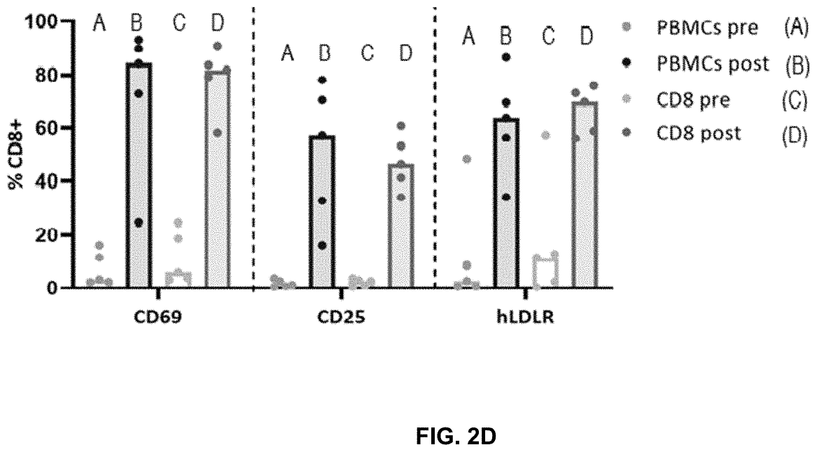

The average number of CD8+ selected cells recovered from a whole leukapheresis bag was about 1×10⁹ (range 7×10⁸-2×10⁹, FIG. 1A) and the average CD8+ T cell purity (of the total live lymphocytes) was 83% (FIG. 1B). PBMC and CD8+ T cells activated with anti-CD3 and anti-CD28 for 24 hours showed no significant difference in the viability (FIG. 2A) and recovery post-activation (FIG. 2B). There was no difference in the frequencies of live lymphocytes, CD3+, CD8+ and CD4+ T cells pre- and post-anti-CD3/CD28 activation in bulk PBMC and CD8+ selected cells (FIG. 2C). This demonstrates that there is no deleterious impact on the accelerated pace of the preparation method or the use of CD8+ cells over a bulk PMBC population.

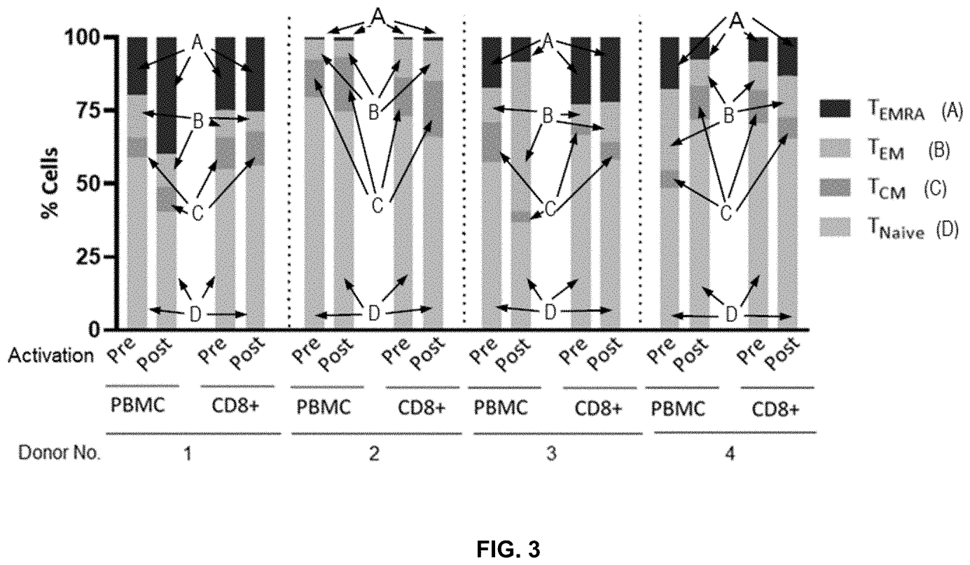

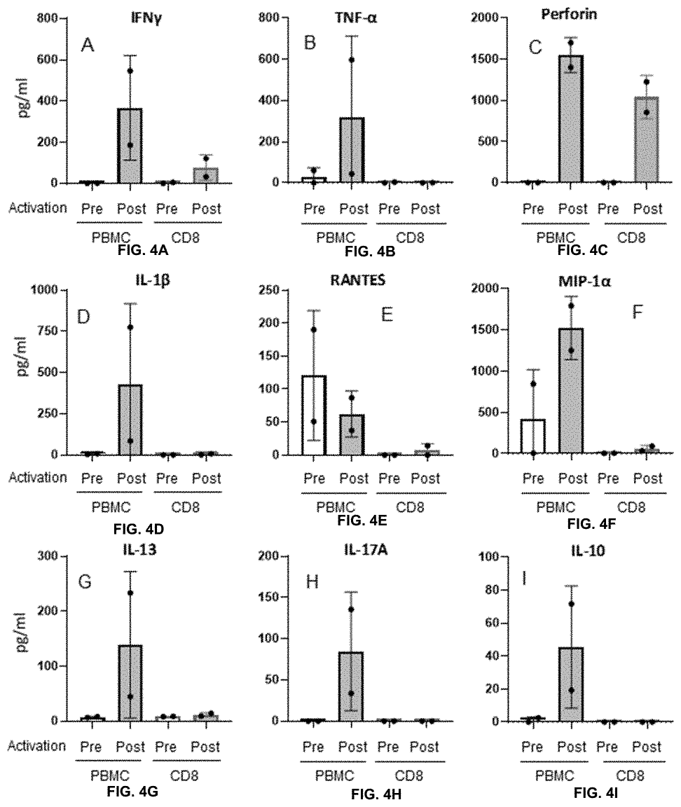

The percentages of CD8+ cells, positive for activation markers, e.g., CD69, CD25, and hLDL-R, obtained from activated PBMC and CD8+ T cells were comparable (FIG. 2D). The distribution of Memory T cell subsets (gated on CD3+CD8+); naïve cells (T_{Naive}), central memory (T_{CM}), effector memory (T_{EM}) and terminally differentiated effector cells (T_{EMRA}), characterized by CCR7+ and CD45RA+ expression, was highly variable amongst different donors but similar between PBMC and CD8+ selected cells per donor (FIG. 3). Anti-CD3/anti-CD28-activated CD8+ cells secreted only cytotoxic (IFN-γ (FIG. 4A), TNF-α (FIG. 4B), Perforin (FIG. 4C)) cytokines compared to activated bulk PBMC that secreted cytotoxic as well as helper cytokines (IL-13 (FIG. 4G), IL-17A (FIG. 4H) and IL-10 (FIG. 4I)), when cultured in complete TexMACS media for 24 hours. TCR-transformed CD8-selected T cells products surprisingly possess higher number of transduced cells compared to TCR-transformed-PBMC T cell products

### Transduction of CD8-Derived and PBMC-Derived T Cells Products

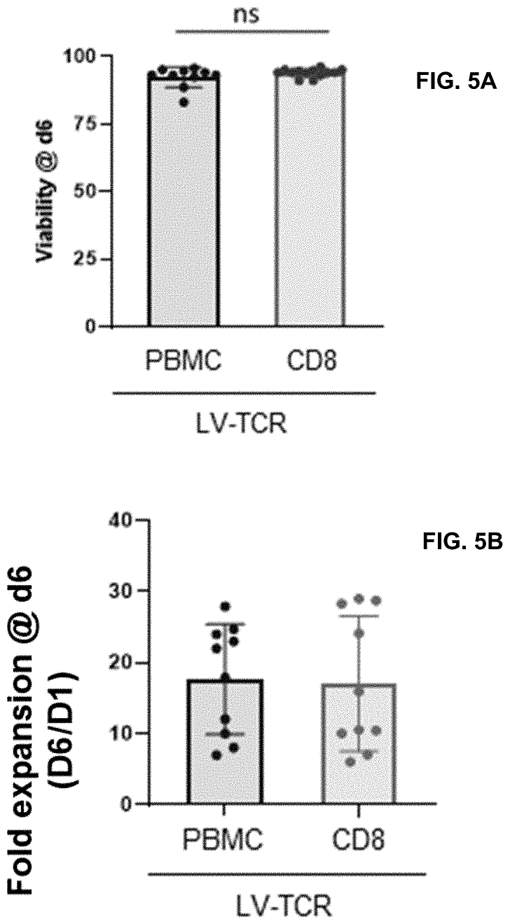

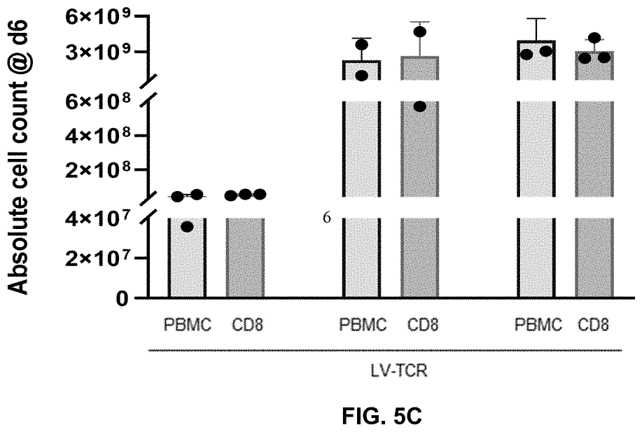

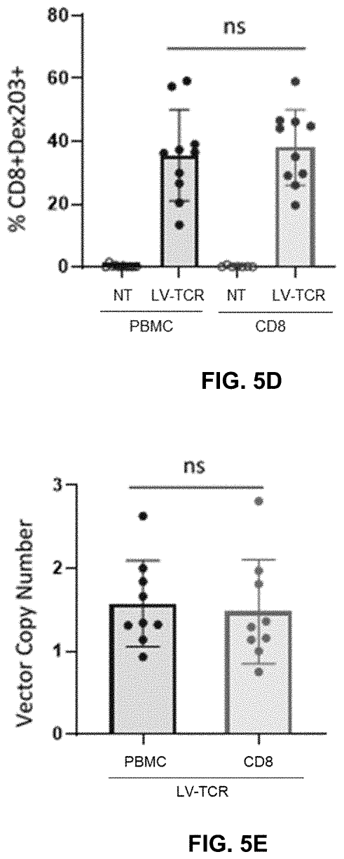

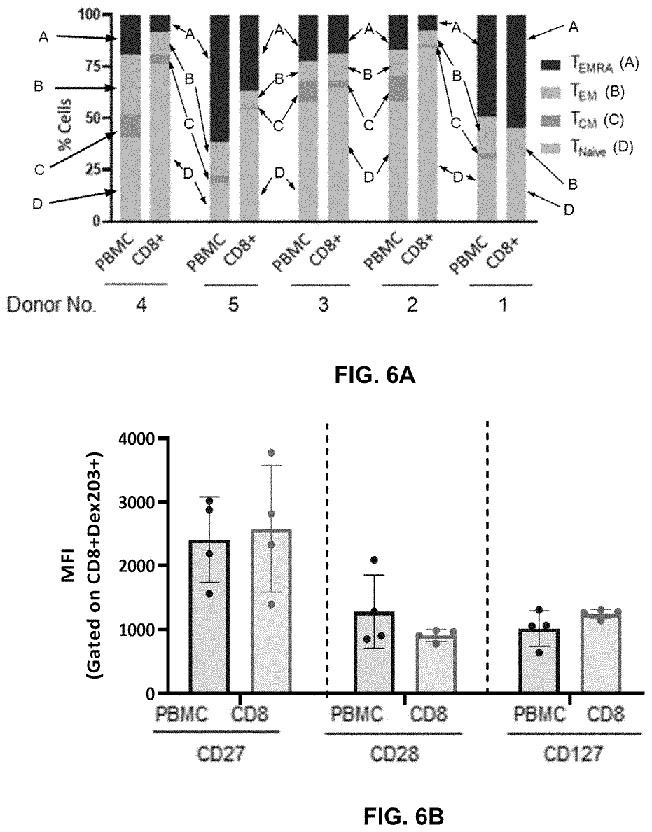

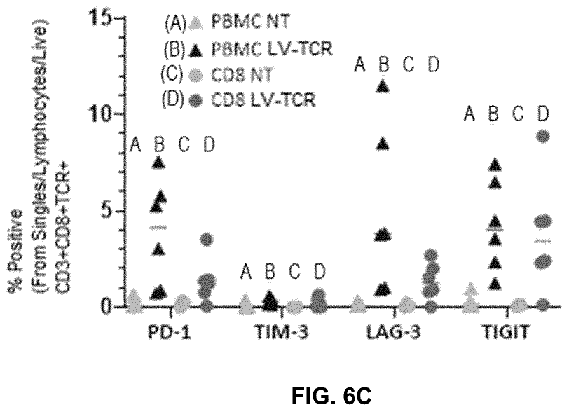

Activated bulk PBMC and CD8+ T cells were transduced with lentivirus encoding a recombinant TCR (LV-TCR) and expanded for 6 days. There was no significant difference in the viabilities (FIG. 5A), fold expansion (FIG. 5B) and absolute counts (FIG. 5C) between PBMC- and CD8-derived LV-TCR-transformed T cell products at day 6. The expression of transgenic TCR, characterized by TCR-specific dextramer (Dex) binding, was similar between PBMC- and CD8-derived LV-TCR-transformed T cell products (FIG. 5D). Likewise, no significant difference was observed in the vector copy number (VCN) between PBMC and CD8-derived LV-TCR-transformed T cell products (FIG. 5E). In 3 out of 5 donors, CD8+Dex+ cells from CD8+ selected LV-TCR-transformed T cell products exhibited improved memory T cell profile, with increased number of less differentiated naïve like and central memory T cells compared to the paired PBMC derived LV-TCR-transformed T cell products (FIG. 6A). However, there was no significant difference in the expression of other memory markers including CD27, CD28 and CD127 on CD8+Dex+ in final product derived from both the cell types (FIG. 6B). In addition, CD8+Dex+ gated from CD8-derived LV-TCR-transformed T cell products had lower PD-1 and LAG-3 expression compared to the PBMC-derived LV-TCR-transformed T cell products (FIG. 6C).

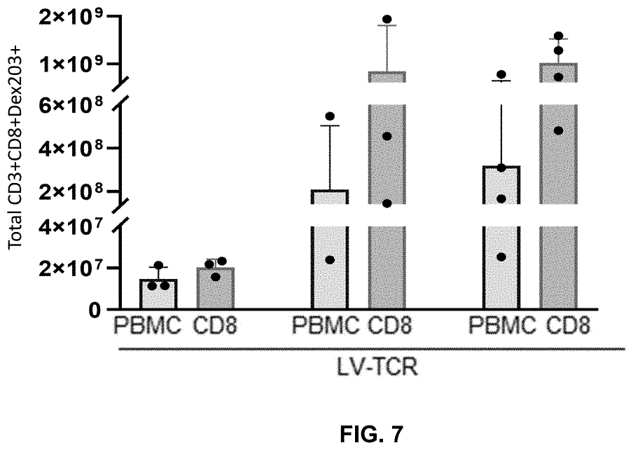

To determine the effect of transducing CD8-selected cells on the scales of T cell manufacturing, PBMC or CD8+ T cells were thawed and then activated with anti-human CD3/CD28 antibodies for 16-20 h and incubated with the transduction mixture containing LV-R11KEA (SEQ ID NO: 162 and SEQ ID NO: 163) for 24 h in the following scales: mid-scale (e.g., transduced cells were seeded and expanded in Grex10/6-well Grex plate), large-scale (e.g., transduced cells were seeded and expanded in Grex100), and GMP-scale (e.g., transduced cells were seeded and expanded in Grex500). Cells were expanded in complete TexMACS with IL-7 and IL-15 for 5 days after transduction. At day 6, i.e., total duration from thaw to harvest is 6 days, PBMC and CD8 products were harvested, cell counts were performed. FACS analysis for PRAME dextramer percentages was performed using T cell memory panel as shown in Table 4. FIG. 7 shows that, most importantly, significantly higher transduced cells, represented by viable CD3+CD8+Dex+ cells in the final CD8-derived LV-TCR-transformed T cell products compared to bulk PBMC at all tested scales, e.g., mid-scale, large-scale, and GMP-scale. Donors with low CD8+ frequencies (e.g., <25%) in the starting PBMC material had significantly higher number of transduced cells (CD3+CD8+Dex+) in the CD8-derived LV-TCR-transformed T cell products compared to in the PBMC-derived LV-TCR-transformed T cell products. This shows that there is no deleterious impact on the transduction of the CD8-selected cells versus PMBC bulk cell population.

### Comparable Anti-Tumor Function

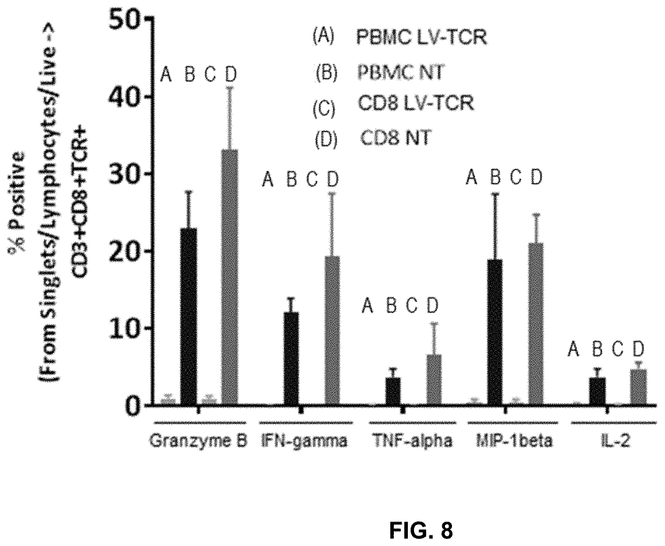

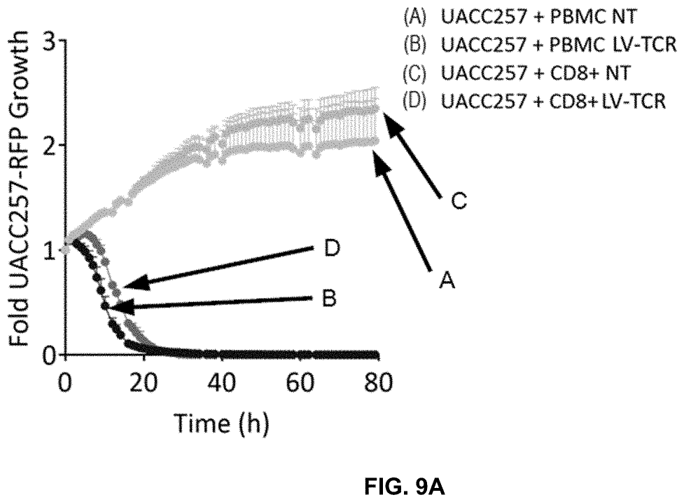

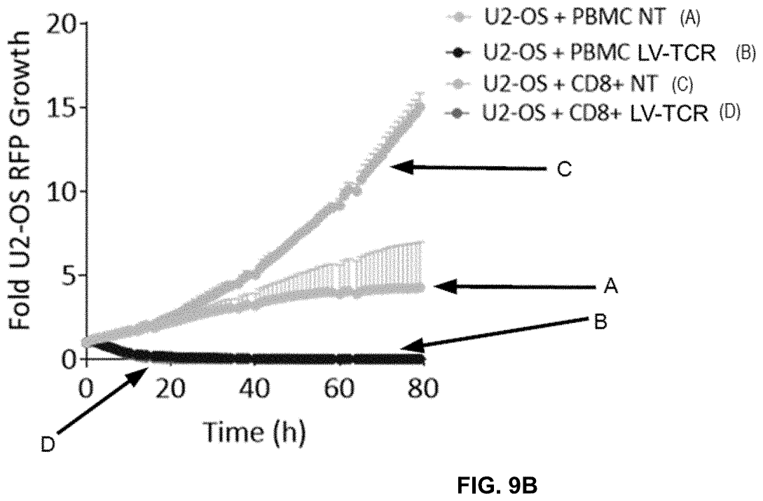

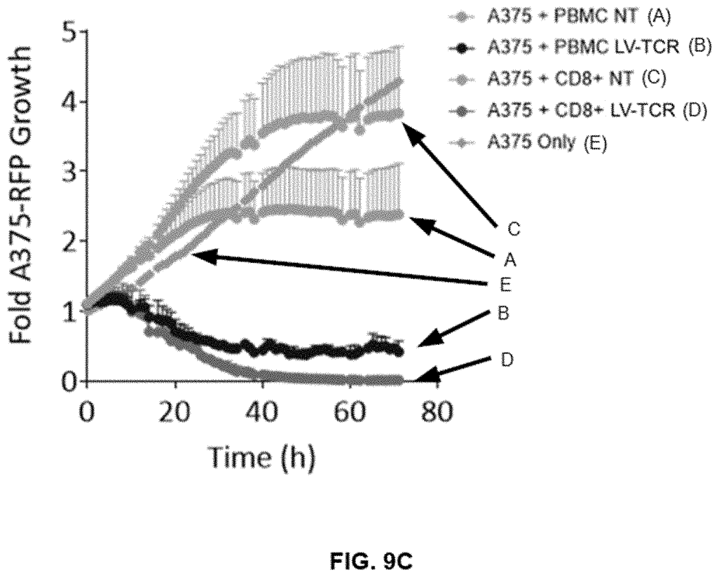

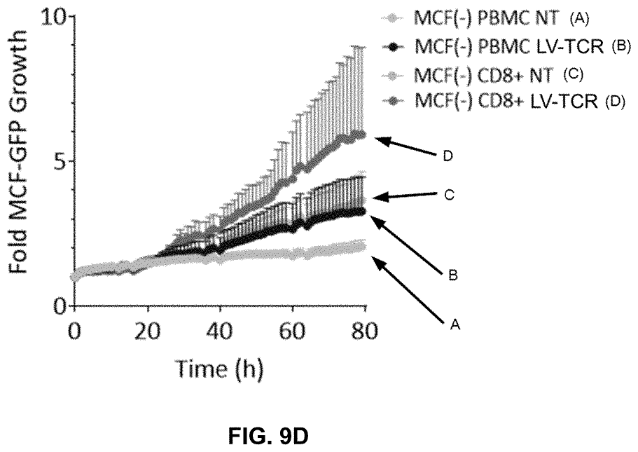

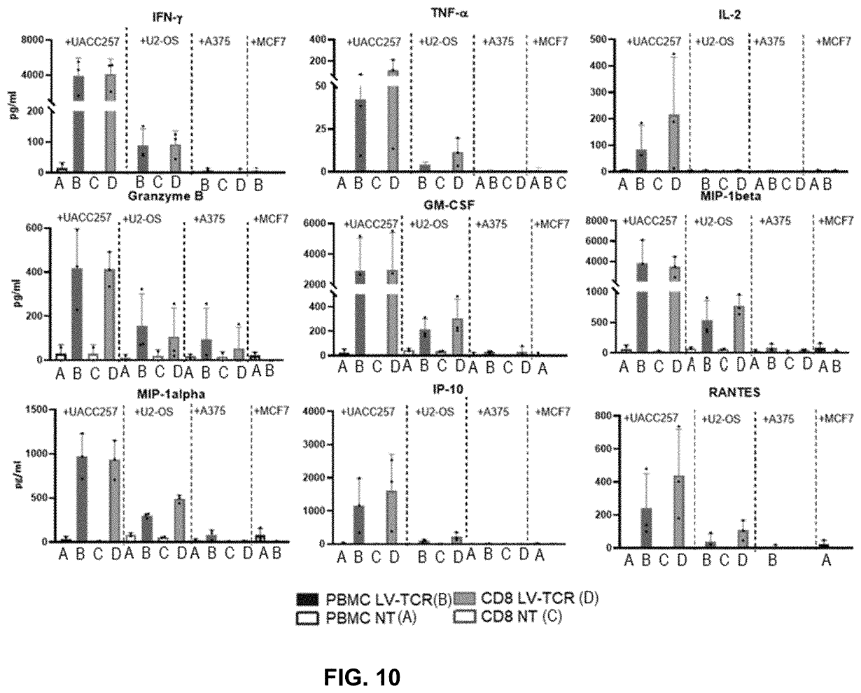

First, to determine the functionality of LV-TCR-transformed-PBMC and LV-TCR-transformed-CD8 T cell products, cytokine response was assessed in response to the HLA-A*02+ target cell line UACC257 by flow cytometry. LV-TCR-transformed T cell products obtained from PBMC or from CD8+ T cells secreted comparable amounts of IFN-γ, Granzyme B, TNF-α, IL-2 and MIP-1β when cocultured with UACC257 tumor cell line (FIG. 8). To further assess the anti-tumor activity of PBMC- and CD8-derived LV-TCR-transformed T cell products, in vitro coculture killing assays were performed and the cytokine-secretion in response to the targets were quantified. PBMC- or CD8+ T cells-derived LV-TCR-transformed T cell products were co-cultured with HLA-A*02+ cell lines expressing varying levels of target antigen (UACC257: ˜1080 copies (FIG. 9A), U2-OS: ˜250 copies (FIG. 9B), A375: ˜50 copies (FIG. 9C), and MCF7: 0 copy (FIG. 9D)) at E:T ratio of 4:1, followed by the tumor growth assessment for 72 hours using an IncuCyte assay. LV-TCR-transformed T cell products derived from both bulk PBMC and CD8+ selected cells showed equivalent killing of the target cells. Additionally, PBMC- or CD8-derived LV-TCR-transformed T cell products secreted comparable amounts of majority of the cytokines including IFN-γ, Granzyme B, GM-CSF, MIP-1α and MIP-1β upon coculture with the target cells after 24 hours (FIG. 10). However, there was a trend towards increased secretion of TNF-α, IL-2, RANTES and IP-10 by CD8-derived LV-TCR-transformed T cell products compared to PBMC-derived LV-TCR-transformed T cell products. The levels of cytokine secretion from PBMC- or CD8-derived LV-TCR-transformed T cell products correlated with the antigen expression on the targets (FIG. 10).

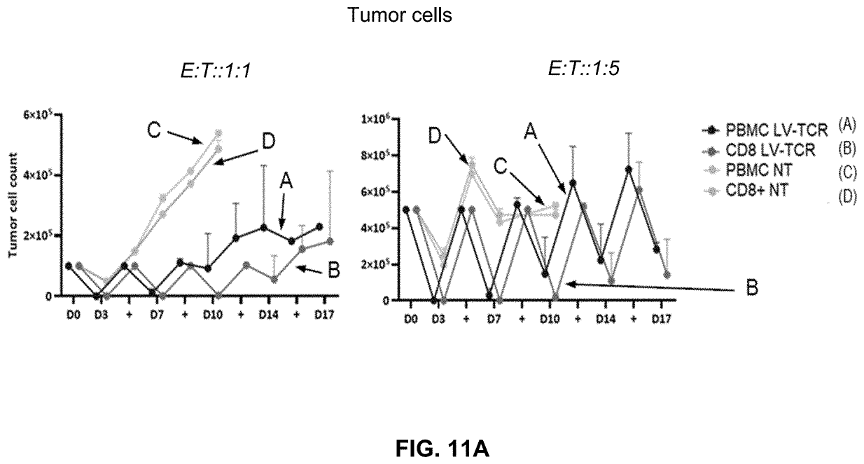

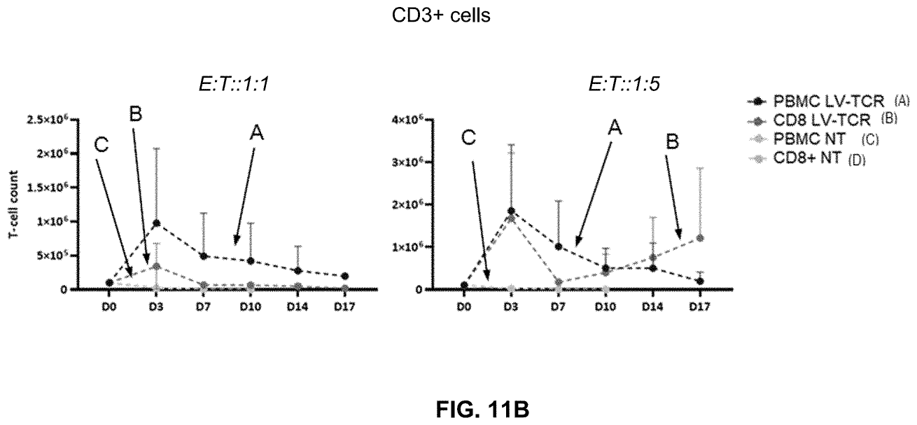

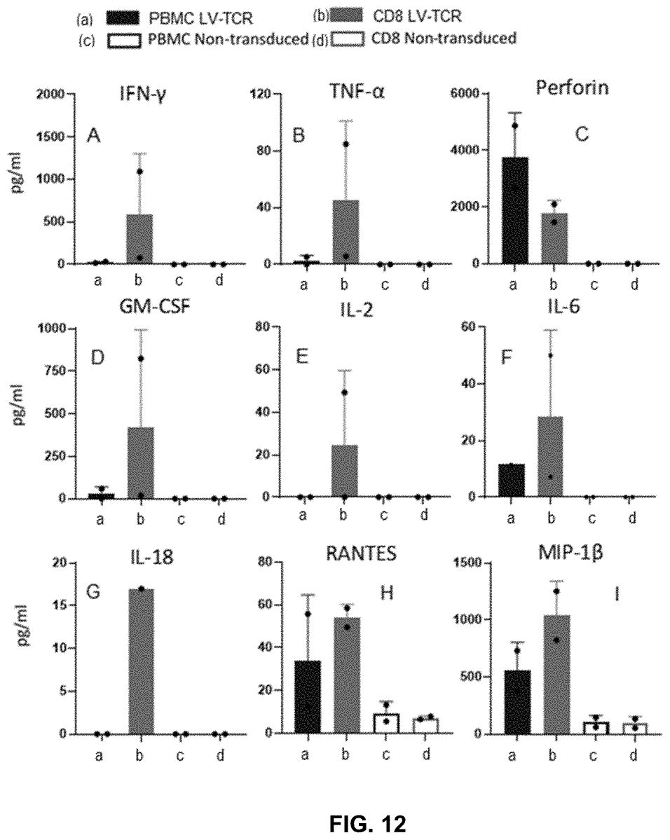

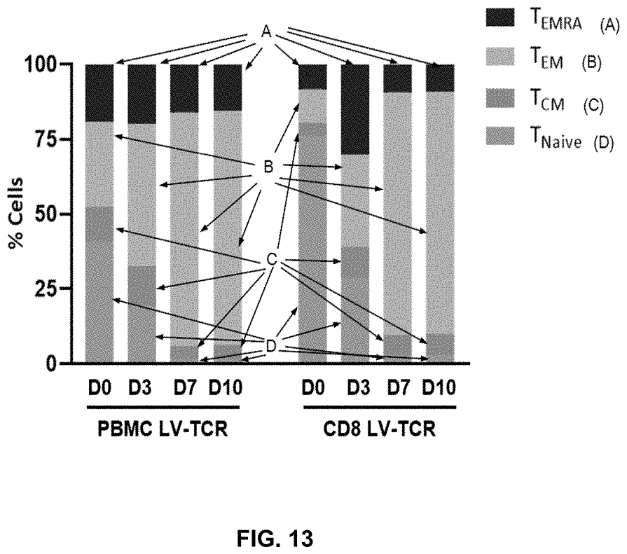

To determine the ability of the LV-TCR-transformed T cell products to kill the target multiple times, long-term serial killing assays were performed. LV-TCR-transformed-PBMC or LV-TCR-transformed-CD8 products were co-cultured with the target cell line THP-1 (Day 0) for 17 days at E:T ratios of 1:1 (FIG. 11A, left panel) and 1:5 (high tumor burden) (FIG. 11A, right panel) followed by residual tumor cells and CD3+ T cells analysis. LV-TCR-transformed-CD8 T cell products were able to suppress tumor growth better than the LV-TCR-transformed-PBMC T cell products at both the tested ratios (3.5 times vs 2.5 times in CD8- and PBMC-derived LV-TCR-transformed T cell products, respectively) at Day 3, Day 7, Day 10, Day 14, and Day 17, when fresh target cells were added back to the assays. Increased CD3+ T cell proliferation was observed in the co-cultures of PBMC-derived LV-TCR-transformed T cell products compared to the CD8-derived LV-TCR-transformed T cell products at the E:T ratio of 1:1 (FIG. 11B, left panel). However, the T cell proliferation was similar in PBMC- and CD8-derived LV-TCR-transformed T cell products at E:T ratio of 1:5 (FIG. 11B, right panel). In addition, increased secretion of majority of the cytokines including IFN-γ, TNF-α, IL-2, IL-18, IL-6, GM-CSF, MIP-1α, and MIP-1β RANTES in the 24 hours co-culture supernatants of CD8-derived LV-TCR-transformed T cell products with the THP-1 cell line at E:T ratios of 1:1 compared to the PBMC-derived product were observed (FIG. 12). Perforin secretion was higher in the PBMC-derived LV-TCR-transformed T cell products compared to the CD8 LV-TCR-transformed T cell products (FIG. 12). Similar trend of cytokine secretion was observed at E:T ratios of 1:5 (data not shown). After every killing, there was an increase in the number of differentiated T cells, T_{EM} and T_{EMRA} in the PBMC- and CD8-derived LV-TCR-transformed T cell products at both the tested ratios. (FIG. 13, data not shown for E:T 1:5).

In sum, these results indicate that there was no difference in the expression of the activation markers on CD8+ derived from bulk PBMC and CD8+ selected T cells. After transduction and expansion, the viabilities, fold expansion, vector copy number and the dextramer binding for recombinant TCR were comparable between the genetically modified T cell products derived from bulk PBMC and CD8+ selected T cells. However, overall genetically modified T cell products had higher number of CD3+CD8+Dextramer203+ cells compared to the product derived from bulk PBMC. Further, both the PBMC- and CD8-derived genetically modified T cell products exhibited comparable short term killing of the tumor cells and secreted comparable amounts of cytokines in response to the tumor targets. However, in the long-term co-culture serial killing assay, CD8-derived genetically modified T cell products exhibited improved ability to kill the target cells multiple times.

This study was conducted to compare the manufacturing and functionality of the TCR-transformed T cell products obtained from bulk PBMC and CD8+ selected T cells as the starting population. The TCR-transformed-CD8 T cell products possessed a higher number of transduced cells (CD3+CD8+Dex+) with comparable or better phenotype and functionality compared to the TCR-transformed-PBMC T cell products.

Advantages of the present invention include, among other advantages, that at all the tested scales, the total transduced cells (CD3+CD8+Dex+) in TCR-transformed-CD8 T cell products are substantially higher compared to the TCR-transformed-PBMC T cell products. The higher number of transduced cells in the CD8-derived product may be attributed to the starting material may be enriched in the CD8+ T cell-content (˜90% of CD3+) as opposed to starting with bulk PBMC, that contained a mixture of CD8+(14-45% of CD3+) and CD4+(32-82% of CD3+) T cells. In addition, as the total number of cells entering manufacturing may be limited to 3 billion PBMC, therefore, on an average only half of the available starting material may be used in the current PBMC based process. Consequently, only half of the available CD8+ T cells in the leukapheresis may be used in manufacturing, which may be especially concerning in patients with low CD8 frequency. On the contrary, performing CD8 selection upfront and initiating manufacturing with CD8+ cells allows for maximum enrichment of target population in the starting material that serve as the starting material for transformed T cell products. Further, the methods described herein circumvent the need to handle large number of cells. The average number of CD8+ cells recovered from a leukapheresis may be about 1 billion, which is well below the maximum capacity of 3 billion as the starting number for the methods described herein. Thus, starting TCR-transformed T cell products manufacturing with CD8+ selected cells may generate a product with higher frequency of functionally transduced cells (CD3+CD8+Dex+). As such, donors with low CD8+ frequency in the starting material (e.g., <25% of CD3+ cells) may have substantially higher number of CD3+CD8+Dex+ cells in the CD8-derived TCR-transformed T cell products compared to that in the bulk PBMC.

The inventors also surprisingly discovered that the CD8+Dex+ cells in the CD8-derived TCR-transformed T cell products comprise a significantly number of higher naïve-like and central memory T cells and exhibit reduced expression of exhaustion markers including PD-1 and LAG-3 as compared to the CD8+Dex+ cells in the PBMC-derived TCR-transformed T cell products. This suggests that CD8-derived TCR-transformed T cell products expectantly possess younger and less differentiated T cell population with less exhausted phenotype capable of persisting longer compared to the PBMC-derived TCR-transformed T cell products. Without being tied to a specific theory, the inventors attributed the unexpected improvement in the quality of the final product to the importance of differences in the milieu created by other cells present in PBMC as compared to when CD8+ T cells are grown separately, which is reflected by differential expression of various cytokines observed post-activation.

The CD8-derived TCR-transformed T cell products show equivalent killing rates of the tumor target cells expressing low, moderate and high levels of PRAME antigen in the short 72 hours coculture killing assay to PMBC-derived products. In addition, the cytokines (IFN-γ, Granzyme A/B, Perforin, GM-CSF, MIP-1α and MIP-1β) secreted after 24 hour coculture with the target cell lines were comparable. Accordingly, the methods herein allow for the rapid, streamlined isolation of CD8+TCR-transformed T cells without compromising the activity of the T cell product.

CD8-derived TCR-transformed T cell products exhibit an unexpected superiority in their capacity to kill the THP-1 target cells multiple times compared to the PBMC-derived TCR-transformed T cell products. The CD8-derived TCR-transformed T cells also surprisingly secreted higher amounts of cytokines after 24 hours in co-culture with THP-1 cell targets. Without being bound to a single theory, a possible explanation is that the CD8-derived T cell products are enriched with CD8+ T cells with the cytotoxic potential and lack any potential immunosuppressive cells, such as Tregs, as compared to the PBMC-derived T cell products. Accordingly, the methods herein allow for the rapid, streamlined isolation of CD8+TCR-transformed T cells with superior properties versus PBMC-derived T cell products.

The inventors also surprisingly discovered that the CD8-derived TCR-transformed T cell products are purer than the PMBC-derived TCR-transformed T cell products because the latter is manufactured from bulk PBMC that contain impurities, such as CD4+ T cells, Tregs, B-cells, monocytes, as compared to the former, which is manufactured from pure CD8+ T cell population. From safety standpoint, there are fewer concerns regarding the CD8-derived TCR-transformed T cell products because their functionality may be comparable with the PMBC-derived TCR-transformed T cell products. Taken together, CD8-derived TCR-transformed T cell products may have less impurities and higher number of functionally transduced cells with very comparable phenotype and anti-tumor function than the PMBC-derived TCR-transformed T cell products. The manufacturing of TCR-transformed T cell products from a CD8+ selected T cells could be advantageous over bulk PBMC when a patient has low CD8+ T cell counts. This could be one option to meet the higher doses of genetically modified T cell products for clinical trials.

### Example 3

### Manufacturing of Tcr-Transformed T Cell Products in Serum Free Medium

Effects of Serum on Transduction in T Cell Manufacturing

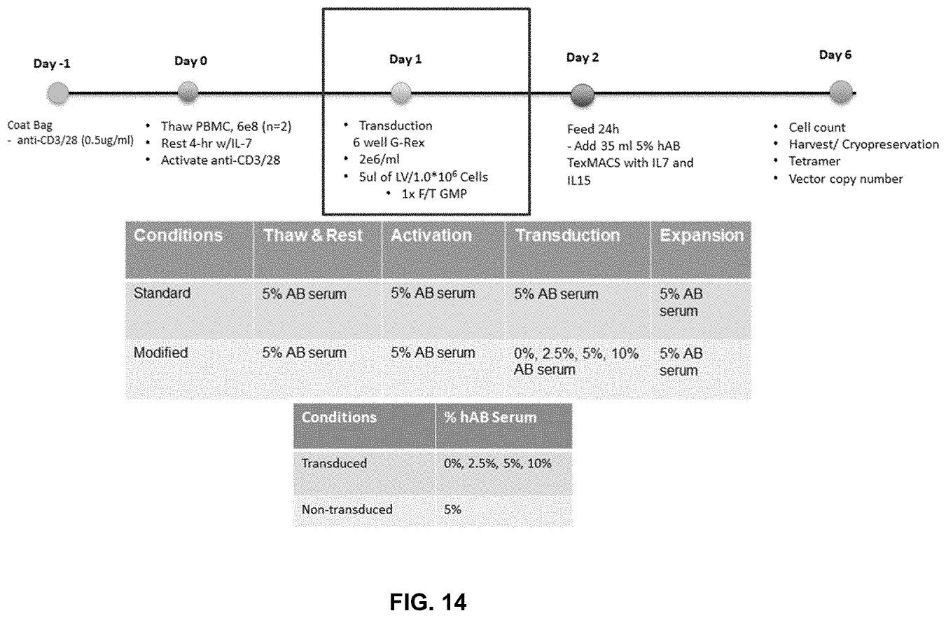

FIG. 14 shows a 6-day manufacturing process for T cells obtained from two donors transduced with a viral vector expressing a TCR targeting MAGEA4/8. To compare the effect of serum in transduction on T cell products, the T cell products generated by the standard condition, in which thaw/rest, activation, transduction, and expansion were performed in 5% human AB (hAB) serum, are compared with that generated by the modified conditions. The modified conditions differ from the standard condition in that the transduction was performed in the presence of 0% (i.e., serum free medium), 2.5%, 5%, or 10% hAB serum in the former and the transduction was performed in the presence 5% hAB in the latter. A control, non-transduction, was performed in the presence of 5% hAB serum.

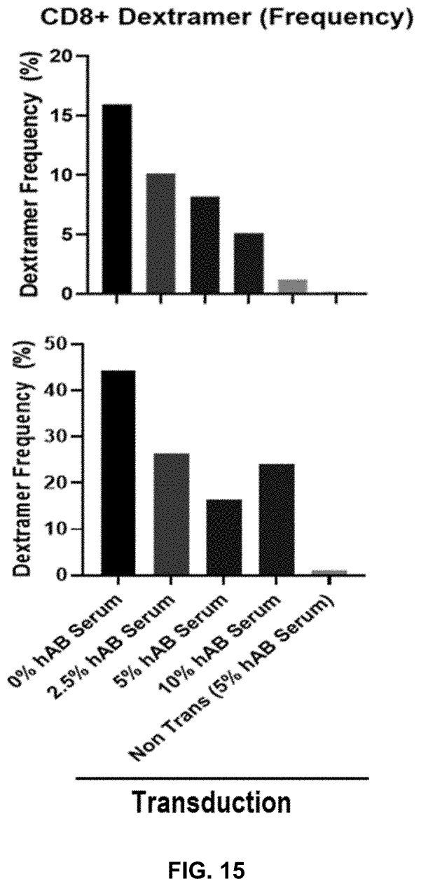

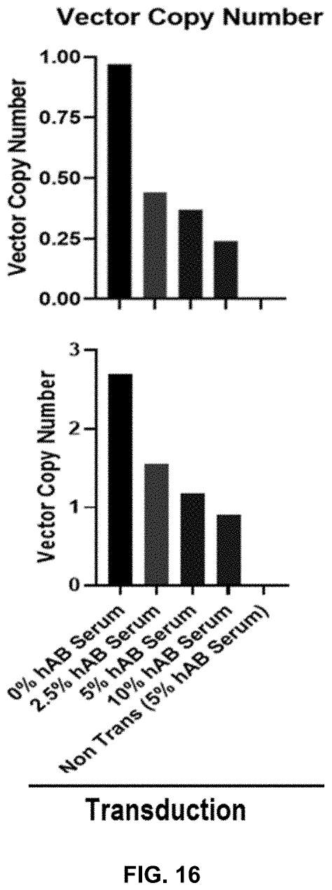

The % of CD8+TCR+ T cell products (% of CD8+Dextramer) obtained from two donors (upper and lower panels) generated by performing transduction in the presence of serum, e.g., 2.5%, 5%, or 10% hAB serum, are lower than that generated by performing transduction in serum free medium, i.e., 0% hAB (FIG. 15). In addition, the vector copy number in T cell products obtained from two donors (upper and lower panels) generated by performing transduction in the presence of serum, e.g., 2.5%, 5%, and 10% hAB serum, are lower than that generated by performing transduction in serum free medium, i.e., 0% hAB, in which the vector copy number remains lower than 5 in compliance with the safety requirements (FIG. 16). These results suggest that transduction performed in serum free medium may be advantageous as indicated by more CD8+TCR+ T cells generated with higher vector copy number as compared with that performed in the presence of serum, e.g., conventional 5% hAB.

Effects of Serum on Activation and Transduction in T Cell Manufacturing

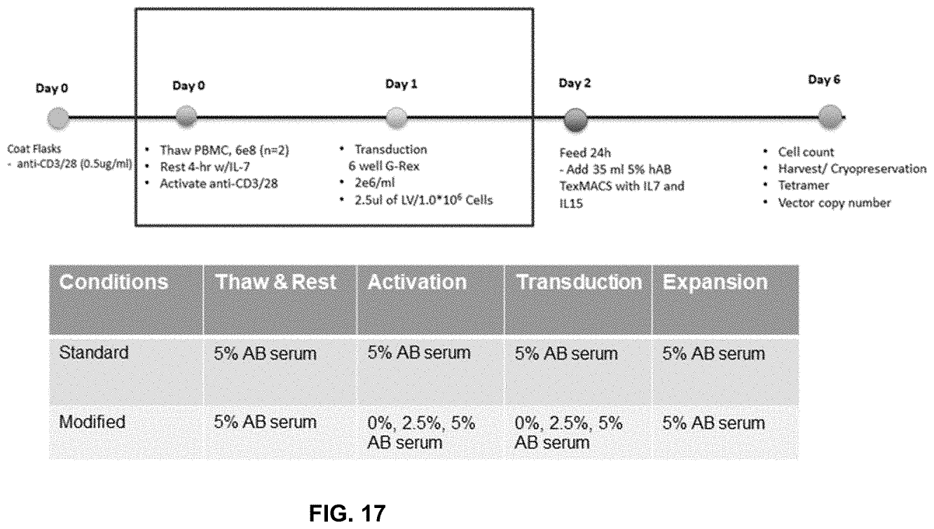

FIG. 17 shows a 6-day manufacturing process for T cells transduced with a viral vector expressing a TCR targeting PRAME. To compare the effect of serum in activation and transduction on T cell products, the T cell products generated by the standard condition, in which thaw/rest, activation, transduction, and expansion were performed in 5% human AB (hAB) serum, are compared with that generated by the modified conditions. The modified conditions differ from the standard condition in that the activation and the transduction were each performed in the presence of 0% (i.e., serum free medium), 2.5%, 5%, or 10% hAB serum in the former and the transduction was performed in the presence 5% hAB in the latter.

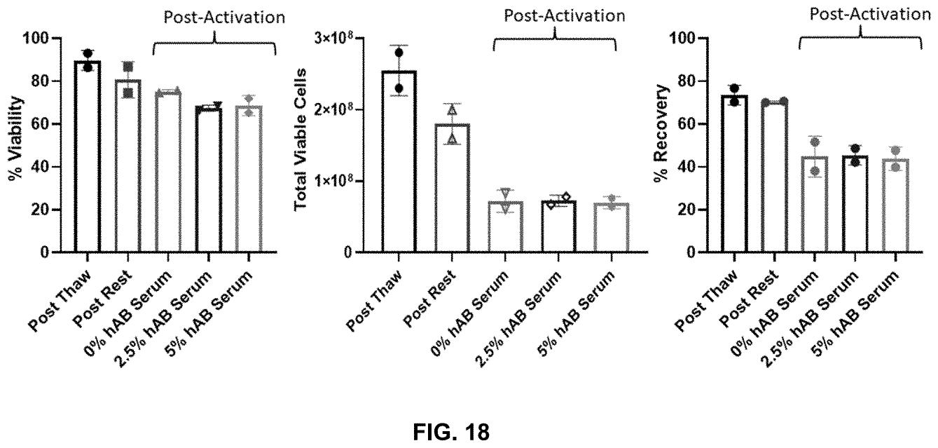

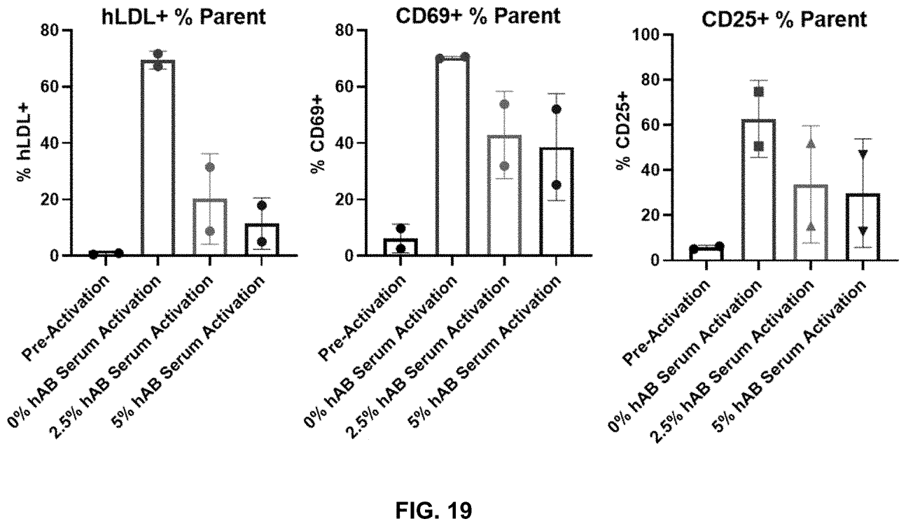

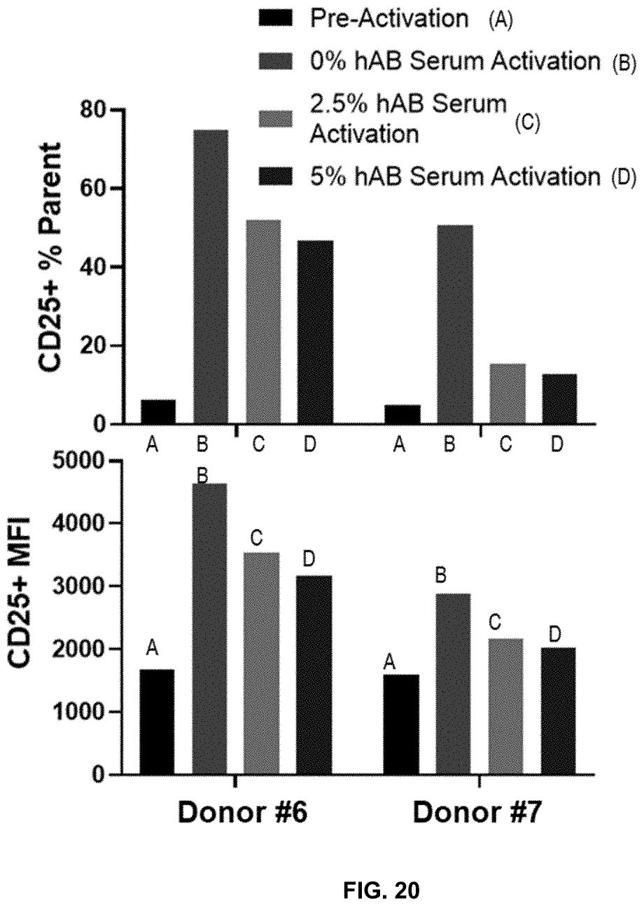

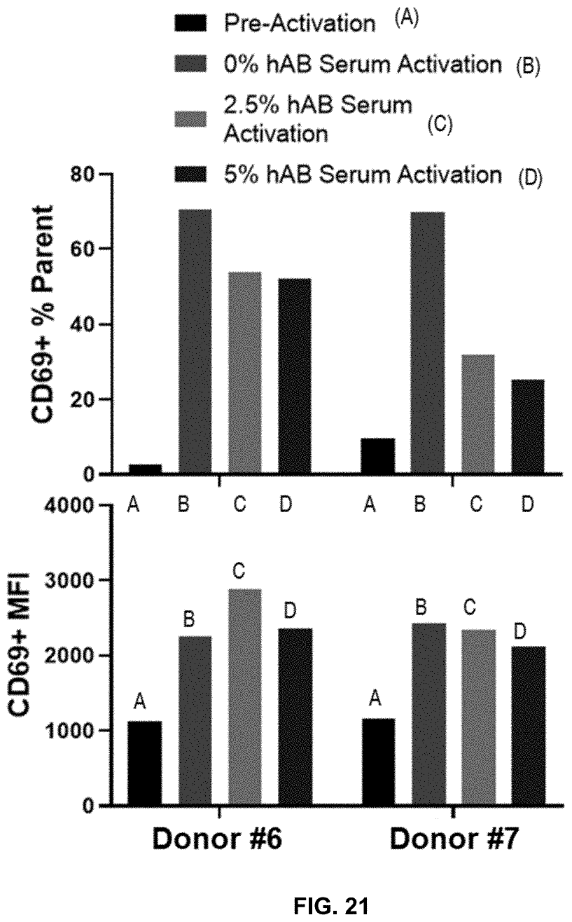

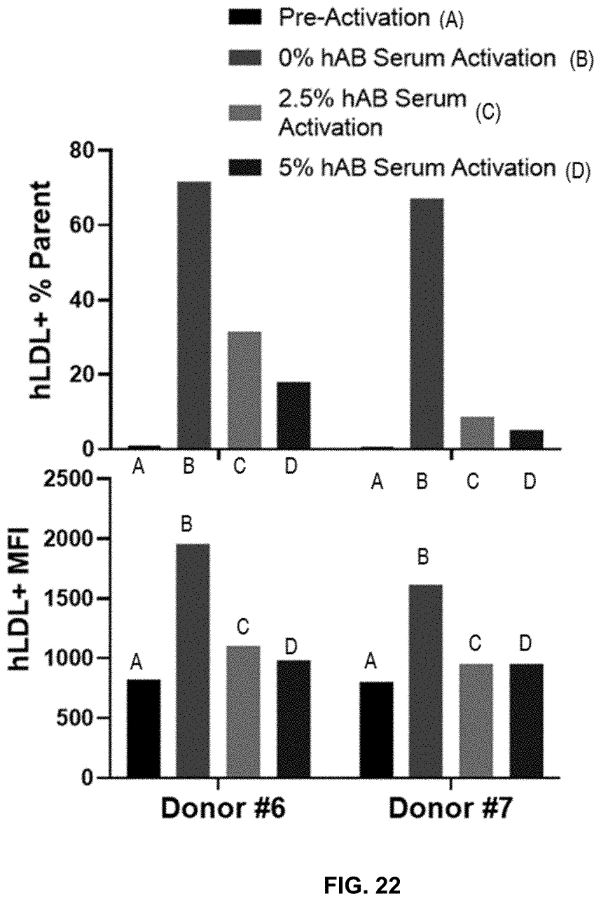

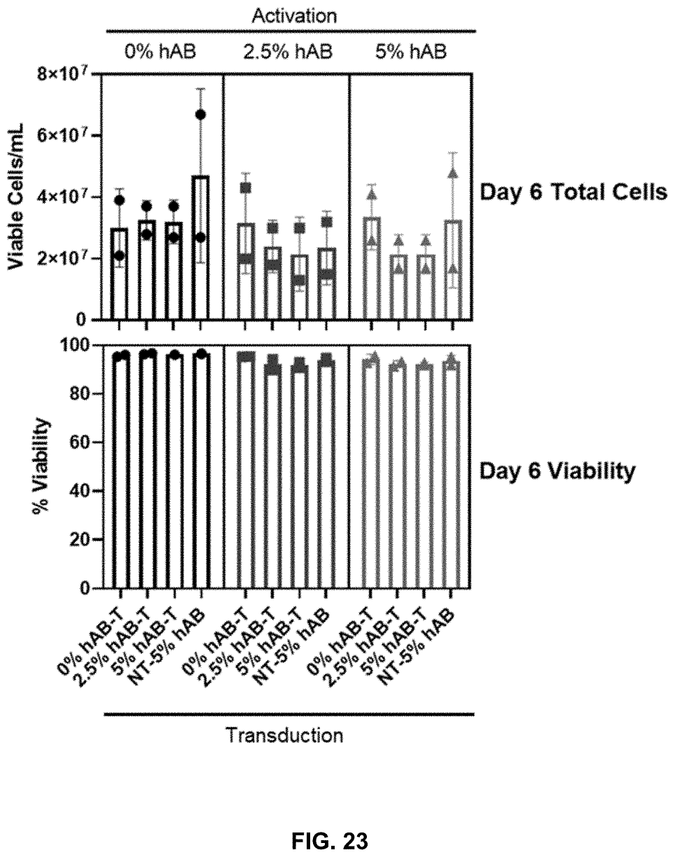

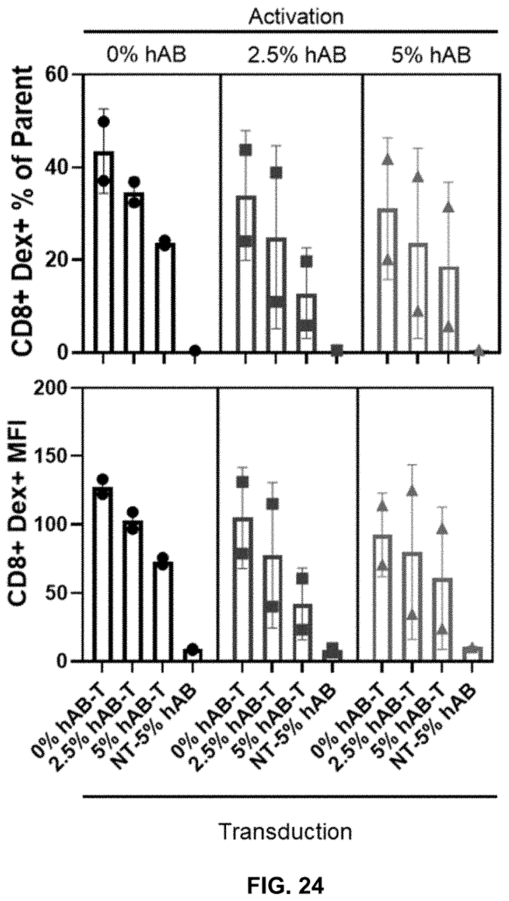

At post-activation, % viability (left panel), total viable cells (middle panel), and % recovery of T cell products (N=2) generated in serum free medium (0% hAB) and in the presence of 2.5% and 5% hAB are comparable (FIG. 18), indicating that activation performed in serum free medium may not compromise cell recovery from activation. In addition, the expression of activation markers, e.g., human low density lipoprotein (hLDL) (left panel), CD69 (middle panel), and CD25 (right panel), of T cell products (N=2) generated in serum free medium (0% hAB) is higher than or comparable to that generated in the presence of serum (FIG. 19). Note that the donor variability in activation marker expression is significantly less than that generated in the presence of serum. Specifically, the amount of CD25+ T cell products (CD25+% parent (FIG. 20, upper panel) and CD25+ MFI (FIG. 20, lower panel)), the amount of CD69+ T cell products (CD69+% parent (FIG. 21, upper panel) and CD69+ MFI (FIG. 21, lower panel)), and the amount of hLDL+ T cell products (hLDL+% parent (FIG. 22, upper panel) and hLDL+ MFI (FIG. 22, lower panel)) from donors generated in serum free medium (0% hAB) are higher than or comparable to that generated in the presence of serum. Furthermore, Day 6 total cells (FIG. 23, upper panel) and Day 6 viability (FIG. 23, lower panel) and CD8+Dex+% parent (FIG. 24, upper panel) and CD8+Dex+ MFI (FIG. 24, lower panel) of T cell products obtained from donors generated in serum free medium (0% hAB) during activation and/or transduction are comparable to that generated in the presence of serum during activation and/or transduction. As noted above, the amount of human AB serum used in the media was identified as a potential rate-limiting step and needed to be reduced to overcome global supply limitations. These results, therefore, support the use of serum free medium in activation and/or transduction because the T cell products thus generated are comparable to that generated in the presence of serum, e.g., conventional 5% hAB.

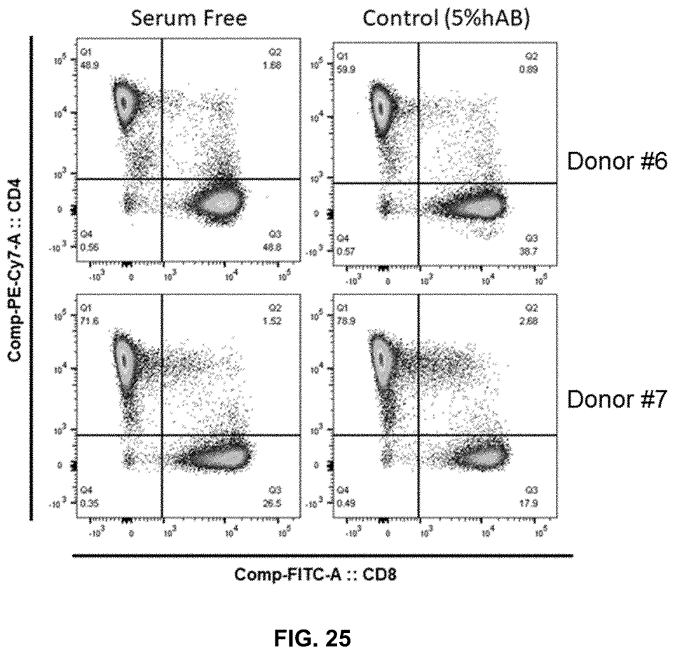

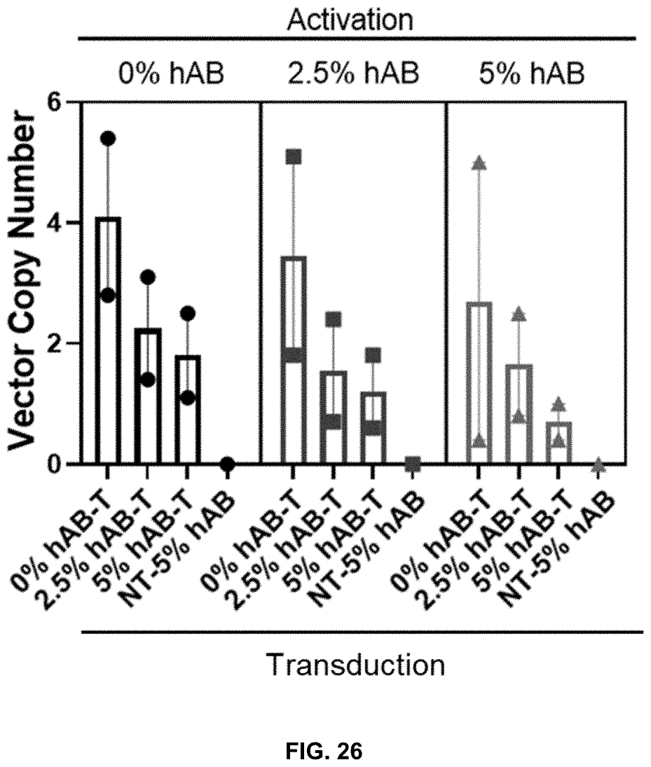

Activation and transduction performed in serum free medium also yielded more CD8+ cells, in which 48.8% CD8+ cells obtained in serum free media and 38.7% CD8+ cells in 5% hAB (Donor #6) and 26.5% CD8+ cells obtained in serum free and 17.9% CD8+ cells obtained in 5% hAB (Donor #7) (FIG. 25). The vector copy number (VCN) in T cell products generated by performing activation and/or transduction in the presence of serum, e.g., 2.5% and 5% serum, are lower than that by performing activation and/or transduction in serum free medium, i.e., 0% hAB, in which the vector copy number remains lower than 5 in compliance with the safety requirements (FIG. 26). These results suggest that activation and/or transduction performed in serum free medium may produce more CD8+ T cell products with higher vector copy number as compared with that performed in the presence of serum.

In sum, serum, e.g., hAB serum, may inhibit transduction efficiency of viral vectors expressing transgenes, e.g., TCRs, during T cell manufacturing. The presence of hAB serum during transduction may have an inhibitory effect on expansion despite complete 5% hAB serum TexMACS media were used for all samples during expansion. The results of flow analysis and vector copy number show a significant improvement in transduction efficiency in the absence of hAB serum as compared to that in the presence of serum, e.g., conventional 5% hAB. In other words, reducing serum during activation may improve transduction efficiency. Activation performed in serum free media may result in the largest and most consistent increase in post-activation marker signals as compared with that performed in the presence of serum. Activation and transduction performed in serum free media may also yield the highest transduction efficiency and may minimize the donor to donor variability, as compared with that performed in the presence of serum, although serum may still be needed for the expansion step

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. A method of producing an engineered T cell population, comprising obtaining a cell population comprising a CD8+ T cell,
isolating the CD8+ T cell from the obtained cell population,
activating the isolated CD8+ T cell,
introducing a nucleic acid encoding a T cell receptor (TCR) binding to an antigen in a complex with an MEW molecule into the activated CD8+ T cell, and
expanding the introduced CD8+ T cell to obtain the engineered T cell population.

2. The method of claim 1, wherein the cell population comprises peripheral blood mononuclear cell (PBMC).

3. The method of claim 2, wherein the PBMC comprises less than 25% of CD8+ cells.

4. The method of claim 1, wherein the isolating comprises contacting the CD8+ cell with an anti-CD8 antibody.

5. The method of claim 1, wherein the activating is performed in the presence of an anti-CD3 antibody and an anti-CD28 antibody.

6. The method of claim 1, wherein the TCR is selected from Table 1.

7. The method of claim 1, wherein the antigen is selected from SEQ ID NO: 1-161.

8. The method of claim 1, wherein the TCR binds to SLLQHLIGL (SEQ ID NO: 50).

9. The method of claim 8, wherein the TCR is selected from R11KEA (SEQ ID NO: 162 and 163), R11P3D3 (SEQ ID NO: 204 and 205), R16P1C10 (SEQ ID NO: 206 and 207), R16P1E8 (SEQ ID NO: 208 and 209), R17P1A9 (SEQ ID NO: 210 and 211), R17P1D7 (SEQ ID NO: 212 and 213), R17P1G3 (SEQ ID NO: 214 and 215), R17P2B6 (SEQ ID NO: 216 and 217), and R11P3D3KE (SEQ ID NO: 218 and 219).

10.-12. (canceled)

13. A method of producing an engineered T cell population, comprising obtaining a cell population comprising a T cell,
resting the obtained cell population,
activating the rested cell population,
introducing a nucleic acid encoding a T cell receptor (TCR) binding to an antigen in a complex with an MEW molecule into the activated cell population in the absence of serum, and
expanding the introduced cell population to obtain the engineered T cell population.

14. The method of claim 13, wherein the cell population comprises peripheral blood mononuclear cell (PBMC).

15. The method of claim 13, wherein the resting is performed for about 2-8 hours, about 2-6 hours, or about 2-4 hours.

16. The method of claim 13, wherein the resting is performed in the presence of serum.

17. The method of claim 13, wherein the activating is performed in the presence of an anti-CD3 antibody and an anti-CD28 antibody.

18. The method of claim 13, wherein the activating is performed in the absence of serum.

19. The method of claim 13, wherein the TCR is selected from Table 1.

20. The method of claim 13, wherein the antigen is selected from SEQ ID NO: 1-161.

21. The method of claim 13, wherein the TCR binds to SLLQHLIGL (SEQ ID NO: 50).

22. The method of claim 21, wherein the TCR is selected from R11KEA (SEQ ID NO: 162 and 163), R11P3D3 (SEQ ID NO: 204 and 205), R16P1C10 (SEQ ID NO: 206 and 207), R16P1E8 (SEQ ID NO: 208 and 209), R17P1A9 (SEQ ID NO: 210 and 211), R17P1D7 (SEQ ID NO: 212 and 213), R17P1G3 (SEQ ID NO: 214 and 215), R17P2B6 (SEQ ID NO: 216 and 217), and R11P3D3KE (SEQ ID NO: 218 and 219).

23. The method of claim 13, wherein the MHC molecule is a class I MHC molecule.

24.-29. (canceled)


SEQUENCE LISTING (240 sequences)

SEQ ID NO: 1 - protein, 10 aa, Homo sapiens
YLYDSETKNA

SEQ ID NO: 2 - protein, 9 aa, Homo sapiens
HLMDQPLSV

SEQ ID NO: 3 - protein, 9 aa, Homo sapiens
GLLKKINSV

SEQ ID NO: 4 - protein, 9 aa, Homo sapiens
FLVDGSSAL

SEQ ID NO: 5 - protein, 10 aa, Homo sapiens
FLFDGSANLV

SEQ ID NO: 6 - protein, 9 aa, Homo sapiens
FLYKIIDEL

SEQ ID NO: 7 - protein, 11 aa, Homo sapiens
FILDSAETTTL

SEQ ID NO: 8 - protein, 9 aa, Homo sapiens
SVDVSPPKV

SEQ ID NO: 9 - protein, 8 aa, Homo sapiens
VADKIHSV

SEQ ID NO: 10 - protein, 9 aa, Homo sapiens
IVDDLTINL

SEQ ID NO: 11 - protein, 9 aa, Homo sapiens
GLLEELVTV

SEQ ID NO: 12 - protein, 10 aa, Homo sapiens
TLDGAAVNQV

SEQ ID NO: 13 - protein, 10 aa, Homo sapiens
SVLEKEIYSI

SEQ ID NO: 14 - protein, 9 aa, Homo sapiens
LLDPKTIFL

SEQ ID NO: 15 - protein, 9 aa, Homo sapiens
YTFSGDVQL

SEQ ID NO: 16 - protein, 9 aa, Homo sapiens
YLMDDFSSL

SEQ ID NO: 17 - protein, 9 aa, Homo sapiens
KVWSDVTPL

SEQ ID NO: 18 - protein, 11 aa, Homo sapiens
LLWGHPRVALA

SEQ ID NO: 19 - protein, 11 aa, Homo sapiens
KIWEELSVLEV

SEQ ID NO: 20 - protein, 9 aa, Homo sapiens
LLIPFTIFM

SEQ ID NO: 21 - protein, 9 aa, Homo sapiens
FLIENLLAA

SEQ ID NO: 22 - protein, 11 aa, Homo sapiens
LLWGHPRVALA

SEQ ID NO: 23 - protein, 9 aa, Homo sapiens
FLLEREQLL

SEQ ID NO: 24 - protein, 9 aa, Homo sapiens
SLAETIFIV

SEQ ID NO: 25 - protein, 9 aa, Homo sapiens
TLLEGISRA

SEQ ID NO: 26 - protein, 9 aa, Homo sapiens
ILQDGQFLV

SEQ ID NO: 27 - protein, 10 aa, Homo sapiens
VIFEGEPMYL

SEQ ID NO: 28 - protein, 9 aa, Homo sapiens
SLFESLEYL

SEQ ID NO: 29 - protein, 9 aa, Homo sapiens
SLLNQPKAV

SEQ ID NO: 30 - protein, 9 aa, Homo sapiens
GLAEFQENV

SEQ ID NO: 31 - protein, 9 aa, Homo sapiens
KLLAVIHEL

SEQ ID NO: 32 - protein, 9 aa, Homo sapiens
TLHDQVHLL

SEQ ID NO: 33 - protein, 11 aa, Homo sapiens
TLYNPERTITV

SEQ ID NO: 34 - protein, 9 aa, Homo sapiens
KLQEKIQEL

SEQ ID NO: 35 - protein, 10 aa, Homo sapiens
SVLEKEIYSI

SEQ ID NO: 36 - protein, 11 aa, Homo sapiens
RVIDDSLVVGV

SEQ ID NO: 37 - protein, 9 aa, Homo sapiens
VLFGELPAL

SEQ ID NO: 38 - protein, 9 aa, Homo sapiens
GLVDIMVHL

SEQ ID NO: 39 - protein, 9 aa, Homo sapiens
FLNAIETAL

SEQ ID NO: 40 - protein, 9 aa, Homo sapiens
ALLQALMEL

SEQ ID NO: 41 - protein, 9 aa, Homo sapiens
ALSSSQAEV

SEQ ID NO: 42 - protein, 11 aa, Homo sapiens
SLITGQDLLSV

SEQ ID NO: 43 - protein, 9 aa, Homo sapiens
QLIEKNWLL

SEQ ID NO: 44 - protein, 9 aa, Homo sapiens
LLDPKTIFL

SEQ ID NO: 45 - protein, 9 aa, Homo sapiens
RLHDENILL

SEQ ID NO: 46 - protein, 9 aa, Homo sapiens
YTFSGDVQL

SEQ ID NO: 47 - protein, 9 aa, Homo sapiens
GLPSATTTV

SEQ ID NO: 48 - protein, 11 aa, Homo sapiens
GLLPSAESIKL

SEQ ID NO: 49 - protein, 9 aa, Homo sapiens
KTASINQNV

SEQ ID NO: 50 - protein, 9 aa, Homo sapiens
SLLQHLIGL

SEQ ID NO: 51 - protein, 9 aa, Homo sapiens
YLMDDFSSL

SEQ ID NO: 52 - protein, 9 aa, Homo sapiens
LMYPYIYHV

SEQ ID NO: 53 - protein, 9 aa, Homo sapiens
KVWSDVTPL

SEQ ID NO: 54 - protein, 11 aa, Homo sapiens
LLWGHPRVALA

SEQ ID NO: 55 - protein, 9 aa, Homo sapiens
VLDGKVAVV

SEQ ID NO: 56 - protein, 9 aa, Homo sapiens
GLLGKVTSV

SEQ ID NO: 57 - protein, 9 aa, Homo sapiens
KMISAIPTL

SEQ ID NO: 58 - protein, 11 aa, Homo sapiens
GLLETTGLLAT

SEQ ID NO: 59 - protein, 9 aa, Homo sapiens
TLNTLDINL

SEQ ID NO: 60 - protein, 9 aa, Homo sapiens
VIIKGLEEI

SEQ ID NO: 61 - protein, 9 aa, Homo sapiens
YLEDGFAYV

SEQ ID NO: 62 - protein, 11 aa, Homo sapiens
KIWEELSVLEV

SEQ ID NO: 63 - protein, 9 aa, Homo sapiens
LLIPFTIFM

SEQ ID NO: 64 - protein, 10 aa, Homo sapiens
ISLDEVAVSL

SEQ ID NO: 65 - protein, 10 aa, Homo sapiens
KISDFGLATV

SEQ ID NO: 66 - protein, 11 aa, Homo sapiens
KLIGNIHGNEV

SEQ ID NO: 67 - protein, 9 aa, Homo sapiens
ILLSVLHQL

SEQ ID NO: 68 - protein, 9 aa, Homo sapiens
LDSEALLTL

SEQ ID NO: 69 - protein, 13 aa, Homo sapiens
VLQENSSDYQSNL

SEQ ID NO: 70 - protein, 11 aa, Homo sapiens
HLLGEGAFAQV

SEQ ID NO: 71 - protein, 9 aa, Homo sapiens
SLVENIHVL

SEQ ID NO: 72 - protein, 9 aa, Homo sapiens
YTFSGDVQL

SEQ ID NO: 73 - protein, 9 aa, Homo sapiens
SLSEKSPEV

SEQ ID NO: 74 - protein, 10 aa, Homo sapiens
AMFPDTIPRV

SEQ ID NO: 75 - protein, 9 aa, Homo sapiens
FLIENLLAA

SEQ ID NO: 76 - protein, 9 aa, Homo sapiens
FTAEFLEKV

SEQ ID NO: 77 - protein, 9 aa, Homo sapiens
ALYGNVQQV

SEQ ID NO: 78 - protein, 9 aa, Homo sapiens
LFQSRIAGV

SEQ ID NO: 79 - protein, 11 aa, Homo sapiens
ILAEEPIYIRV

SEQ ID NO: 80 - protein, 9 aa, Homo sapiens
FLLEREQLL

SEQ ID NO: 81 - protein, 10 aa, Homo sapiens
LLLPLELSLA

SEQ ID NO: 82 - protein, 9 aa, Homo sapiens
SLAETIFIV

SEQ ID NO: 83 - protein, 11 aa, Homo sapiens
AILNVDEKNQV

SEQ ID NO: 84 - protein, 9 aa, Homo sapiens
RLFEEVLGV

SEQ ID NO: 85 - protein, 9 aa, Homo sapiens
YLDEVAFML

SEQ ID NO: 86 - protein, 11 aa, Homo sapiens
KLIDEDEPLFL

SEQ ID NO: 87 - protein, 9 aa, Homo sapiens
KLFEKSTGL

SEQ ID NO: 88 - protein, 11 aa, Homo sapiens
SLLEVNEASSV

SEQ ID NO: 89 - protein, 10 aa, Homo sapiens
GVYDGREHTV

SEQ ID NO: 90 - protein, 10 aa, Homo sapiens
GLYPVTLVGV

SEQ ID NO: 91 - protein, 9 aa, Homo sapiens
ALLSSVAEA

SEQ ID NO: 92 - protein, 9 aa, Homo sapiens
TLLEGISRA

SEQ ID NO: 93 - protein, 9 aa, Homo sapiens
SLIEESEEL

SEQ ID NO: 94 - protein, 9 aa, Homo sapiens
ALYVQAPTV

SEQ ID NO: 95 - protein, 10 aa, Homo sapiens
KLIYKDLVSV

SEQ ID NO: 96 - protein, 9 aa, Homo sapiens
ILQDGQFLV

SEQ ID NO: 97 - protein, 9 aa, Homo sapiens
SLLDYEVSI

SEQ ID NO: 98 - protein, 9 aa, Homo sapiens
LLGDSSFFL

SEQ ID NO: 99 - protein, 10 aa, Homo sapiens
VIFEGEPMYL

SEQ ID NO: 100 - protein, 9 aa, Homo sapiens
ALSYILPYL

SEQ ID NO: 101 - protein, 9 aa, Homo sapiens
FLFVDPELV

SEQ ID NO: 102 - protein, 11 aa, Homo sapiens
SEWGSPHAAVP

SEQ ID NO: 103 - protein, 9 aa, Homo sapiens
ALSELERVL

SEQ ID NO: 104 - protein, 9 aa, Homo sapiens
SLFESLEYL

SEQ ID NO: 105 - protein, 9 aa, Homo sapiens
KVLEYVIKV

SEQ ID NO: 106 - protein, 10 aa, Homo sapiens
VLLNEILEQV

SEQ ID NO: 107 - protein, 9 aa, Homo sapiens
SLLNQPKAV

SEQ ID NO: 108 - protein, 9 aa, Homo sapiens
KMSELQTYV

SEQ ID NO: 109 - protein, 11 aa, Homo sapiens
ALLEQTGDMSL

SEQ ID NO: 110 - protein, 11 aa, Homo sapiens
VIIKGLEEITV

SEQ ID NO: 111 - protein, 9 aa, Homo sapiens
KQFEGTVEI

SEQ ID NO: 112 - protein, 9 aa, Homo sapiens
KLQEEIPVL

SEQ ID NO: 113 - protein, 9 aa, Homo sapiens
GLAEFQENV

SEQ ID NO: 114 - protein, 9 aa, Homo sapiens
NVAEIVIHI

SEQ ID NO: 115 - protein, 9 aa, Homo sapiens
ALAGIVTNV

SEQ ID NO: 116 - protein, 12 aa, Homo sapiens
NLLIDDKGTIKL

SEQ ID NO: 117 - protein, 10 aa, Homo sapiens
VLMQDSRLYL

SEQ ID NO: 118 - protein, 9 aa, Homo sapiens
KVLEHVVRV

SEQ ID NO: 119 - protein, 9 aa, Homo sapiens
LLWGNLPEI

SEQ ID NO: 120 - protein, 9 aa, Homo sapiens
SLMEKNQSL

SEQ ID NO: 121 - protein, 9 aa, Homo sapiens
KLLAVIHEL

SEQ ID NO: 122 - protein, 10 aa, Homo sapiens
ALGDKFLLRV

SEQ ID NO: 123 - protein, 11 aa, Homo sapiens
FLMKNSDLYGA

SEQ ID NO: 124 - protein, 10 aa, Homo sapiens
KLIDHQGLYL

SEQ ID NO: 125 - protein, 12 aa, Homo sapiens
GPGIFPPPPPQP

SEQ ID NO: 126 - protein, 9 aa, Homo sapiens
ALNESLVEC

SEQ ID NO: 127 - protein, 9 aa, Homo sapiens
GLAALAVHL

SEQ ID NO: 128 - protein, 9 aa, Homo sapiens
LLLEAVWHL

SEQ ID NO: 129 - protein, 9 aa, Homo sapiens
SIIEYLPTL

SEQ ID NO: 130 - protein, 9 aa, Homo sapiens
TLHDQVHLL

SEQ ID NO: 131 - protein, 9 aa, Homo sapiens
SLLMWITQC

SEQ ID NO: 132 - protein, 11 aa, Homo sapiens
FLLDKPQDLSI

SEQ ID NO: 133 - protein, 10 aa, Homo sapiens
YLLDMPLWYL

SEQ ID NO: 134 - protein, 9 aa, Homo sapiens
GLLDCPIFL

SEQ ID NO: 135 - protein, 9 aa, Homo sapiens
VLIEYNFSI

SEQ ID NO: 136 - protein, 11 aa, Homo sapiens
TLYNPERTITV

SEQ ID NO: 137 - protein, 9 aa, Homo sapiens
AVPPPPSSV

SEQ ID NO: 138 - protein, 9 aa, Homo sapiens
KLQEELNKV

SEQ ID NO: 139 - protein, 11 aa, Homo sapiens
KLMDPGSLPPL

SEQ ID NO: 140 - protein, 9 aa, Homo sapiens
ALIVSLPYL

SEQ ID NO: 141 - protein, 9 aa, Homo sapiens
FLLDGSANV

SEQ ID NO: 142 - protein, 10 aa, Homo sapiens
ALDPSGNQLI

SEQ ID NO: 143 - protein, 9 aa, Homo sapiens
ILIKHLVKV

SEQ ID NO: 144 - protein, 9 aa, Homo sapiens
VLLDTILQL

SEQ ID NO: 145 - protein, 9 aa, Homo sapiens
HLIAEIHTA

SEQ ID NO: 146 - protein, 9 aa, Homo sapiens
SMNGGVFAV

SEQ ID NO: 147 - protein, 9 aa, Homo sapiens
MLAEKLLQA

SEQ ID NO: 148 - protein, 9 aa, Homo sapiens
YMLDIFHEV

SEQ ID NO: 149 - protein, 11 aa, Homo sapiens
ALWLPTDSATV

SEQ ID NO: 150 - protein, 9 aa, Homo sapiens
GLASRILDA

SEQ ID NO: 151 - protein, 9 aa, Homo sapiens
ALSVLRLAL

SEQ ID NO: 152 - protein, 9 aa, Homo sapiens
SYVKVLHHL

SEQ ID NO: 153 - protein, 9 aa, Homo sapiens
VYLPKIPSW

SEQ ID NO: 154 - protein, 9 aa, Homo sapiens
NYEDHFPLL

SEQ ID NO: 155 - protein, 9 aa, Homo sapiens
VYIAELEKI

SEQ ID NO: 156 - protein, 12 aa, Homo sapiens
VHFEDTGKTLLF

SEQ ID NO: 157 - protein, 9 aa, Homo sapiens
VLSPFILTL

SEQ ID NO: 158 - protein, 9 aa, Homo sapiens
HLLEGSVGV

SEQ ID NO: 159 - protein, 9 aa, Homo sapiens
ALREEEEGV

SEQ ID NO: 160 - protein, 10 aa, Homo sapiens
KEADPTGHSY

SEQ ID NO: 161 - protein, 9 aa, Homo sapiens
TLDEKVAEL

SEQ ID NO: 162 - protein, 273 aa, Artificial Sequence
  R11KEA alpha chain
MEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 163 - protein, 311 aa, Artificial Sequence
  R11KE beta chain
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 164 - protein, 274 aa, Artificial Sequence
  R20P1H7 alpha chain
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQGENSGYSTLTFGKGTMLLVSPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 165 - protein, 314 aa, Artificial Sequence
  R20P1H7 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGPGLAAYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 166 - protein, 272 aa, Artificial Sequence
  R7P1D5 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEYSSASKIIFGSGTRLSIRPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 167 - protein, 310 aa, Artificial Sequence
  R7P1D5 beta chain
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRANTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 168 - protein, 277 aa, Artificial Sequence
  R10P2G12 alpha chain
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEGNSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 169 - protein, 313 aa, Artificial Sequence
  R10P2G12 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLSSGSHQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 170 - protein, 271 aa, Artificial Sequence
  R10P1A7 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESKETRLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 171 - protein, 317 aa, Artificial Sequence
  R10P1A7 beta chain
MLLLLLLLGPGISLLLPGSLAGSGLGAWSQHPSVWICKSGTSVKIECRSLDFQATTMFWYRQFPKQSLMLMATSNEGSKATYEQGVEKDKFLINHASLTLSTLTVTSAHPEDSSFYICSARAGGHEQFFGPGTRLTVLEDLKNVFPPEVAVFEPSEAEISHTQKATLVCLATGFYPDHVELSWVWNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 172 - protein, 271 aa, Artificial Sequence
  R4P1D10 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFHDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 173 - protein, 308 aa, Artificial Sequence
  R4P1D10 beta chain
MGFRLLCCVAFCLLGAGPVDSGVTQTPKHLITATGQRVTLRCSPRSGDLSVYWYQQSLDQGLQFLIHYYNGEERAKGNILERFSAQQFPDLHSELNLSSLELGDSALYFCASSVASAYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 174 - protein, 274 aa, Artificial Sequence
  R4P3F9 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAYSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 175 - protein, 308 aa, Artificial Sequence
  R4P3F9 beta chain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVESSYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 176 - protein, 271 aa, Artificial Sequence
  R4P3H3 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKAGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 177 - protein, 314 aa, Artificial Sequence
  R4P3H3 beta chain
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLLTSGGDNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 178 - protein, 270 aa, Artificial Sequence
  R36P3F9 alpha chain
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATVSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 179 - protein, 314 aa, Artificial Sequence
  R36P3F9 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSTSGGLSGETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 180 - protein, 272 aa, Artificial Sequence
  R52P2G11 alpha chain
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVSAYGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 181 - protein, 312 aa, Artificial Sequence
  R52P2G11 beta chain
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGSPDGNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 182 - protein, 279 aa, Artificial Sequence
  R53P2A9 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYNSYAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 183 - protein, 310 aa, Artificial Sequence
  R53P2A9 beta chain
MGPGLLCWVLLCLLGAGPVDAGVTQSPTHLIKTRGQQVTLRCSPISGHKSVSWYQQVLGQGPQFIFQYYEKEERGRGNFPDRFSARQFPNYSSELNVNALLLGDSALYLCASSLDGTSEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 184 - protein, 271 aa, Artificial Sequence
  R26P1A9 alpha chain
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCLIGASGSRLTFGEGTQLTVNPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 185 - protein, 310 aa, Artificial Sequence
  R26P1A9 beta chain
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSYFGWNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 186 - protein, 276 aa, Artificial Sequence
  R26P2A6 alpha chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSDVSGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 187 - protein, 311 aa, Artificial Sequence
  R26P2A6 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASTTPDGTDEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 188 - protein, 274 aa, Artificial Sequence
  R26P3H1 alpha chain
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDMNRDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 189 - protein, 310 aa, Artificial Sequence
  R26P3H1 beta chain
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSRAEGGEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 190 - protein, 273 aa, Artificial Sequence
  R35P3A4 alpha chain
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASPTGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 191 - protein, 311 aa, Artificial Sequence
  R35P3A4 beta chain
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSLGGASQEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 192 - protein, 266 aa, Artificial Sequence
  R37P1C9 alpha chain
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILFNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 193 - protein, 309 aa, Artificial Sequence
  R37P1C9 beta chain
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSSGETNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 194 - protein, 277 aa, Artificial Sequence
  R37P1H1 alpha chain
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFGYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 195 - protein, 311 aa, Artificial Sequence
  R37P1H1 beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSNEGQGWEAEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 196 - protein, 268 aa, Artificial Sequence
  R42P3A9 alpha chain
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVHNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 197 - protein, 322 aa, Artificial Sequence
  R42P3A9 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLLGQGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 198 - protein, 276 aa, Artificial Sequence
  R43P3F2 alpha chain
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSNNNAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 199 - protein, 323 aa, Artificial Sequence
  R43P3F2 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPTGTSGYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 200 - protein, 273 aa, Artificial Sequence
  R43P3G5 alpha chain
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALNRDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 201 - protein, 311 aa, Artificial Sequence
  R43P3G5 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASRLPSRTYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 202 - protein, 270 aa, Artificial Sequence
  R59P2E7 alpha chain
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVNSDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 203 - protein, 321 aa, Artificial Sequence
  R59P2E7 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGLGTGDYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 204 - protein, 273 aa, Artificial Sequence
  R11P3D3 alpha chain
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 205 - protein, 311 aa, Artificial Sequence
  R11P3D3 beta chain
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATLLYEILLGKATLYAVLVSALVLMAMVKRKDSRG

SEQ ID NO: 206 - protein, 275 aa, Artificial Sequence
  R16P1C10 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAVISNFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 207 - protein, 311 aa, Artificial Sequence
  R16P1C10 beta chain
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPWDSPNEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 208 - protein, 274 aa, Artificial Sequence
  R16P1E8 alpha chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSEAAGNKLTFGGGTRVLVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 209 - protein, 309 aa, Artificial Sequence
  R16P1E8 beta chain
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSYTNQGEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 210 - protein, 273 aa, Artificial Sequence
  R17P1A9 alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVLNQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 211 - protein, 314 aa, Artificial Sequence
  R17P1A9 beta chain
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSAETGPWLGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 212 - protein, 277 aa, Artificial Sequence
  R17P1D7 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRWAQGGSEKLVFGKGTKLTVNPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 213 - protein, 313 aa, Artificial Sequence
  R17P1D7 beta chain
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCATELWSSGGTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 214 - protein, 273 aa, Artificial Sequence
  R17P1G3 alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVGPSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 215 - protein, 311 aa, Artificial Sequence
  R17P1G3 beta chain
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPGGSGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 216 - protein, 274 aa, Artificial Sequence
  R17P2B6 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVVSGGGADGLTFGKGTHLIIQPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 217 - protein, 319 aa, Artificial Sequence
  R17P2B6 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLGRGGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 218 - protein, 273 aa, Artificial Sequence; identical to SEQ ID NO: 162
  R11P3D3KE alpha chain

SEQ ID NO: 219 - protein, 243 aa, Artificial Sequence
  R11P3D3KE beta chain
NNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATLLYEILLGKATLYAVLVSALVLMAMVKRKDSRG

SEQ ID NO: 220 - protein, 223 aa, Artificial Sequence
  R39P1C12 alpha chain
TYLYWYKQEPGAGLQLLTYIFSNMDMKQDQRLTVLLNKKDKHLSLRIADTQTGDSAIYFCAEIDNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 221 - protein, 307 aa, Artificial Sequence
  R39P1C12 beta chain
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSQLNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 222 - protein, 270 aa, Artificial Sequence
  R39P1F5 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 223 - protein, 311 aa, Artificial Sequence
  R39P1F5 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGANEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 224 - protein, 272 aa, Artificial Sequence
  R40P1C2 alpha chain
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYLNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 225 - protein, 311 aa, Artificial Sequence
  R40P1C2 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEMTAVGQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 226 - protein, 271 aa, Artificial Sequence
  R41P3E6 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAFSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 227 - protein, 310 aa, Artificial Sequence
  R41P3E6 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSQYTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 228 - protein, 270 aa, Artificial Sequence
  R43P3G4 alpha chain
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNGGDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 229 - protein, 311 aa, Artificial Sequence
  R43P3G4 beta chain
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGQGALEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 230 - protein, 281 aa, Artificial Sequence
  R44P3B3 alpha chain
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGLYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 231 - protein, 311 aa, Artificial Sequence
  R44P3B3 beta chain
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSLGDRGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 232 - protein, 272 aa, Artificial Sequence
  R44P3E7 alpha chain
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEINNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 233 - protein, 320 aa, Artificial Sequence
  R44P3E7 beta chain
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSPPDQNTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 234 - protein, 273 aa, Artificial Sequence
  R49P2B7 alpha chain
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVRIFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 235 - protein, 312 aa, Artificial Sequence
  R49P2B7 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLMGELTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 236 - protein, 276 aa, Artificial Sequence
  R55P1G7 alpha chain
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMMGDTGTASKLTFGTGTRLQVTLDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 237 - protein, 309 aa, Artificial Sequence
  R55P1G7 beta chain
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFGGYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 238 - protein, 270 aa, Artificial Sequence
  R59P2A7 alpha chain
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVQPHDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 239 - protein, 307 aa, Artificial Sequence
  R59P2A7 beta chain
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSGLVAEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 240 - protein, 9 aa, Homo sapiens
KIQEILTQV